(19) United States
(12) Patent Application Publication
(10) Pub. No.: US 2011/0039808 A1
(43) Pub. Date: Feb. 17, 2011

(54) Multitarget Compounds Active at a PPAR and Cannabinoid Receptor

(76) Inventors: Pierre Desreumaux, Marq En Baroeul (FR); Salvatore Bellinvia, Pordenone (IT); Philippe Chavatte, Gondecourt (FR); Sergio Baroni, Villa D'adda (IT)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/746,008

(22) PCT Filed: Dec. 22, 2008
(86) PCT No.: PCT/EP08/68205

§ 371 Date: Oct. 29, 2010

(30) Foreign Application Priority Data
Dec. 21, 2007 (IE) 2007/0928

(51) Int. Cl. A61K 31/625; C07D 215/54; C07D 209/12; C07D 209/26; A61P 29/00

(52) U.S. Cl. 514/166; 546/156; 548/493; 548/503

(57) ABSTRACT

There is a need for pharmaceutical compounds which have activity at, at least one of a PPAR and a cannabinoid receptor. Thus there are provided such compounds, wherein the compound comprises: a PPAR pharmacophore and a cannabinoid pharmacophore linked together by a moiety comprising a fused bicyclic ring comprising a five membered ring fused with a six membered ring or a six membered ring fused with a six membered ring; wherein the cannabinoid pharmacophore comprises the fused bicyclic ring; and the PPAR pharmacophore comprises a salicylic acid, alkoxybenzylacetic acid or a alkoxyphenylacetic acid functionality; and wherein the PPAR pharmacophore is linked to the bicyclic ring of the cannabinoid pharmacophore through a linker comprising an amine or an amide functional group.

### FIELD OF THE INVENTION

The invention relates to the provision of compounds which have target activity on at least one receptor. More particularly, the invention relates to pharmaceutical compounds that have multitarget ability, for example, compounds which are simultaneously active on more than one receptor.

### BACKGROUND TO THE INVENTION

Pharmaceutical compounds having targeted activity on at least one receptor are highly desired. Particularly of interest are new compounds which are more potent than existing compounds known to be active at, at least one receptor.

Furthermore, it is now the general consensus that a single drug which interacts with only a single target cannot correct a complex disease such as cancer, diabetes, infectious or immuno-inflammatory diseases. In this context, a compound displaying Multi Target capability would provide an enhancement of efficacy and/or an improvement of safety compared to the present one-drug-one-target methods. The Multi Target approach involves two potential approaches, the first being the combination of several independent compounds that each independently interact with only one specific target, and the second being utilising a single compound that interacts simultaneously with more than one (multiple) target. The combination approach is generally less favoured in so far as it may lead to pharmacokinetics, toxicity and patient compliance problems, often associated with drug combination dose regimes.¹¹⁻¹⁵ Thus the single compound Multi Target approach is preferred.

Design of single chemical compounds that simultaneously modulate multiple biological targets in a specific manner (Multi Target Ligands or MTLs) is the focus of study in the area known as polypharmacology. In fact, the idea of MTL drugs is becoming more popular. One reason for this popularity increase stems from the fact that the disadvantage of increased complexity and cost of design of such drugs is outweighed by benefits such as lower risk of toxicity to the patient and lower treatment costs. In general therapy utilising a single drug is favoured over drug combination therapy. In particular, the reduced likelihood of adverse drug-drug interactions, when compared to current drug cocktail dose regimens or multi-component drug therapy, is favourable. MTLs are required to have pharmacological activity profiles capable of addressing a particular disease. MTLs aim to achieve both enhanced pharmacological efficacy and improved safety by reducing drug cocktail consumption, thereby producing less adverse side effects. MTLs are intended to be selective and ideally will not possess activity against targets of non-interest.

Typically, identification of MTLs arise from either a knowledge based approach or an existing compound screening approach. The knowledge-based approach begins with existing pharmacological data taken from literature sources or other such knowledge banks and compounds are synthesized to contain pharmacophores based on the existing knowledge. A initial stage of high throughput or focused screening involving a large range of structurally diverse compounds for activity at one target, followed by further follow up analysis for activity at a different target, can sometimes result in the opportune identification of compounds displaying a degree of activity at both targets. However, gaps in the knowledge base are a problem that can lead to uncertainty as to where to begin and it is commonly found that based on such an approach an incorrect choice of compounds for screening analysis is made. In practice such methods are quite crude. Indeed it is well accepted in the art that successful use of such methods relies mainly on the fortuitous identification of compounds displaying a desired activity at more than one (both) target. In practice it is significantly rare for this method to lead to a suitable compound which acts as an MTL.

An alternative approach is to take existing individual compounds, each known to have high selectivity against the particular targets of interest. The known pharmacological structural features of each of the individual compounds can then be combined into a single molecule. In these types of methods, existing pharmacological Structure-Activity Relationships (SARs) are very useful and are a means by which the effect of a drug on a particular target can be related to its molecular structure. Structure-Activity Relationships may be assessed by considering a series of molecules and making gradual changes to them, noting the effect of each discreet change on their biological activity. Alternatively, it may be possible to assess a large body of toxicity data using intelligent tools such as neural networks to try to establish a structure/activity relationship. Ideally, such relationships can be formulated as Quantitative Structure Activity Relationships (QSARs), in which some degree of predictive capability is present. The process of introducing known SARs to a compound in the hope of introducing a second activity is known as “designing in”. It may be the case that compound of interest shows activity at an undesirable target. In such a case “designing out” to avoid the undesired activity then becomes important. A drawback however is that designing out oftentimes can deleteriously affect the desired activity, for example, by causing a reduction in activity or an unbalancing of activity against the target receptors of interest. It is well known in the art, that even very small changes to a compound structure may have a big impact on pharmacological function. Thus, the high levels of associated unpredictability are problematic, even with the SAR approach. This is because even in the SAR approach not all interactions are predictable and thus, successful multi-target compound identification still falls, to an extent, to chance rather than being based entirely on predictive analysis. Thus, the reality remains that the identification of MTL compounds which retain target affinity for more than one receptor is extremely difficult and often cannot be achieved at all for a desired functionality. This results in a significant problem, as the provision of a range of MTL drugs is hindered by the inability to predict final activity.

Where SAR information is available for particular compounds the individual molecules containing the active pharmacophores are sometimes linked together by an appropriate cleavable or non-cleavable spacer to form a MTL comprising cleavable or non-cleavable conjugated pharmacophores. Such MTLs are known as “conjugates”. In such an arrangement, a linker group that is not usually found in either individual molecule separates the active pharmacophores. The ligands within the MTL compound act individually at each target site. The linker is generally stable to metabolization. Alternatively, if the linker is designed to be metabolized, the MTL compound is known as a “cleavable conjugate” and release of the two target compounds that interact independently with each target occurs on metabolization. When linkers of decreasing size are employed, the molecular pharmacophores come into closer and closer proximity, until eventually the pharmacophores are essentially touching and the individual compounds can be considered fused. Common structural feature may overlap to provide molecules comprising slightly overlapped pharmacophores, or may be highly merged, wherein the individual pharmacophores are essentially integrated.¹²

Peroxisome proliferator-activated receptors (PPARs) are members of the nuclear receptor superfamily of transcription factors, most of which are ligand dependent transcriptional activators.¹ Three types of PPARs have been identified: alpha, γ and delta. Each of the PPAR subtypes function as a lipid sensor that modulate important metabolic events by co-ordinately upregulating the expression of large gene arrays implicated in glucose and fat metabolism, with each displaying distinct physiological and pharmacological functions depending on their target genes and their tissue distribution. Moreover, PPARs, particularly PPAR-γ and PPAR-α, negatively regulate inflammatory mediator expression in both the periphery and brain. They also have anti-oxidant actions and modulate the proliferation, differentiation, survival and function of immune cells, including macrophages, B cells and T cells, suggesting that PPAR ligands have intrinsic anti-inflammatory actions. Studies performed in vivo have shown that PPARs activation in macrophages, T and B lymphocytes, and epithelial cells suppress the inflammatory response by attenuating the production of chemokines and cytokines secretions. As a consequence, PPARs, particularly PPAR-γ due to its demonstrated anti-atherosclerosic effects, are currently among the most pursued drug targets in the treatment of not only metabolic (e.g. type 2 diabetes mellitus and atherosclerosis) but also CNS (e.g. multiple sclerosis, stroke and chronic neurodegenerative diseases, such as Parkinson's and Alzheimer's diseases) disorders that have an inflammatory component. PPAR activation has been shown to suppress pain² induced behaviour in mice suffering from chemical induced tissue injury, nerve damage, or inflammation.³ High levels of PPARs expression have been reported in both colonic and adipose tissue. Colon epithelial cells and to a lesser degree macrophages and lymphocytes are a major source of PPARs expression.^{4,5} Many compounds are known to be selective towards each PPAR subtypes (PPARγ, PPARα, PPARδ), for example, rosiglitazone, an anti-diabetic drug from the thiazolidinedione class, shows selectivity towards PPARγ, but has no PPARα-binding action. Typical PPAR active, drug related side-effects, include weight gain and fluid retention. It is desirable to avoid these side-effects and one solution would be to use drugs having multi activity against more than one PPAR subtypes. Thus multi target PPAR agonists are desirable since they would be expected to produce less side effects, and doses required may be smaller. A limited number of such MTL drugs are known. Anti-inflammatory drugs such as mesalazine (also known as mesalamine or 5-aminosalicylic acid) which is used to treat inflammation of the digestive tract (Crohn's disease) and mild to moderate ulcerative colitis are known as selective dual agonists of the PPARα and γ. The anti-diabetic drug, rosiglitazone, a thiazolidinedione, on the other hand is a selective ligand of PPARγ, and has no PPARα-binding action.

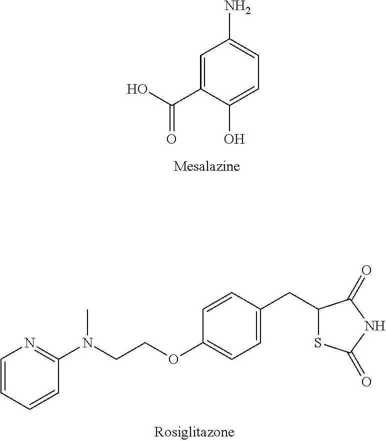

Another thiazolidinedione compound, KRP-297 (see below), was the first target balanced dual PPAR-γ, PPAR-α agonist to be identified and made. It was developed through screening troglitazone (a thiazolidine derivate with PPAR-γ agonist activity), in in vivo models of hyperglycemia and hyperlipidemia in genetically obese mice. Additional target balanced MTLs are highly desired.

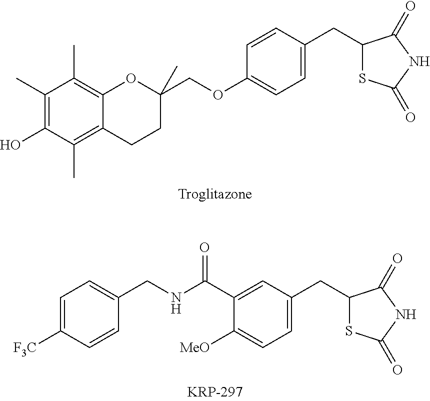

International Publication No. WO 2007/087448 describes a class of spiro imidazole derivatives which have the ability to act as PPAR modulators. The spiro compounds may be useful for the treatment or prevention of diseases or disorders associated with the activity of the Peroxisome Proliferator-Activated Receptor (PPAR) families. The spiro compounds disclosed do not comprise fused ring systems, particularly fused bicyclic ring systems.

The CB₂ receptor is a member of the membranar cannabinoid receptor superfamily. CB₂ receptor is mainly expressed on immune cells such as macrophages, B and T cells, epithelial cells but it is also expressed on myenteric plexus longitudinal muscle (cannabinoid—CB receptor pharmacology is currently the subject of intense academic and commercial research endeavours). Two cannabinoid receptors have been cloned, CB1 and CB2. These Gi/o protein-coupled receptors are distributed throughout the body and are involved in the control of miscellaneous physiological processes, such as pain perception, inflammation, appetite and vasoregulation. CB1 receptors are predominantly found on nerve terminals in the central (CNS) and peripheral (PNS) nervous systems, although they have also been localized in non-neuronal tissues, such as spleen and immunocytes. The primary location of CB2 receptors is on immunocytes, but they have also been identified on peripheral nerves and in the CNS. In addition, certain cannabinoids interact with an orphan receptor GPR55 (G protein receptor). This receptor, together with other non-CB receptors, might account for the considerable pharmacological and functional evidence for the existence of additional targets for endogenous, synthetic and plant-derived cannabinoid ligands (see below).

Recently, attention has turned to identification of CB₂ selective compounds with focus on CB₂ control of pain and inflammation. In particular, active compounds which lack psychoactive effects are of interest. CB₂ selective ligands are effective in animal models of hyperalgesia and inflammation (TNBS- and DSS-induced colitis, carrageen-induced acute inflammation, cerulein-induced acute pancreatitis, Freud Adjuvant-induced inflammatory pain, formalin rat hind paws induced inflammation, hepatic-ischemia reperfusion, LPS-induced chronic brain inflammation, amyotrophic lateral sclerosis (ALS) mouse model, CCL4-induced liver fibrosis).⁶ There have been increasing numbers of reported cannabinoid actions that do not appear to be mediated by either CB₁ or CB₂, the known cannabinoid receptors.⁷ One such example is the synthetic analogue ajulemic acid (AJA, CT-3, IP-751 (see below)), a classical cannabinoid, which shows potent analgesic and anti-inflammatory effects in rodents and humans and is thought not to be mediated by either CB1 or CB2.

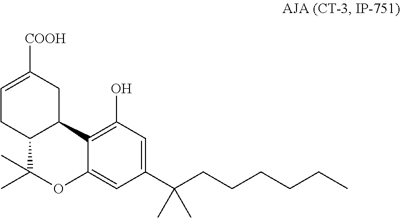

At present, a plethora of cannabinoid ligands have been developed with fairly high selectivity for CB1 and CB2 receptors. At the same time, medicinal indications of CB2 ligands have expanded markedly, based on increasing knowledge in the functioning of the endocannabinoid system in different tissues, herein including the CBS. Although formerly considered as an exclusively peripheral receptor, it is now accepted that the CB2 receptor is also present in limited amounts and distinct locations in the brain of several animal species including humans. Furthermore, the inducible nature of the CB2 receptors under neuro-inflammatory conditions, in contrast to the psychoactive CB1 receptors, makes the non-psychoactive CB2 receptors attractive targets for the development of novel therapeutic approaches. Emerging targets of ligands directed to the CB2 receptor include (neuro)inflammation and pain and, as a consequence, stroke, brain trauma, multiple sclerosis and chronic neurodegenerative diseases, such as Alzheimer's disease and others.

Most recently, it has been reported that cannabinoids/endocannabinoids are activators of not only PPAR-α but also PPAR-γ. Furthermore a variety of small molecule ligands, including AJA, have been shown to induce the activation of PPARs. It has been suggested that PPARs may act as receptor for certain cannabinoid ligands.⁸ This may apply to AJA (CT-3, IP-751) above also. In fact, in addition to evidences showing that the pharmacological effects of endocannabinoid-like substances, such as OEA and PEA, occur in a PPARα-dependant manner, there is now evidence that the endocannabinoids anandamide and 2-arachidonoylglycerol have anti-inflammatory properties mediated in part by PPAR-γ. Recently, Russo et al. have demonstrated that combined use (not in an MTL) of the cannabinoid receptor agonist, anandamide, and the PPAR-α agonist, GW7647, may result in synergistic antinociception (an increased tolerance to pain).⁹ Similarly, ajulemic acid, a synthetic derivative of THC ineffective on CB1/2 receptors, exhibits anti-pain and anti-inflammatory effects in vivo through PPAR-γ. THC and other synthetic CBs (HU210, WIN55212-2 and CP55940) also activate PPAR-γ, with THC leading to a time-dependent vasorelaxation in isolated arteries. On the other hand, PPAR-α agonists, such as thiazolidinediones (eg. Ciglitazone), are able to inhibit, although at high concentrations in vitro, the activity of fatty acid aminohydrolase (FAAH), the main endocannabinoid-degrading enzyme. The possible existence of down-stream overlapping pharmacological mechanisms for compounds acting on PPARs or CBs raises the intriguing possibility of synergistic effects of molecules targeting both CB and PPAR-γ receptors.

### SUMMARY OF THE INVENTION

In light of the foregoing it is desirable to provide new pharmaceutical compounds which have the ability to target at least one type of receptor. New compounds which have more potent activity on at least one receptor are highly advantageous for the reasons provided earlier.

It would be even more advantageous to provide MTL compounds that can simultaneously act on and target more than one receptor. Of particular interest are such MTL compounds which target and are active on at least one PPAR type and at least one of the cannabinoid receptors. It would be particularly useful to do this with balanced receptor activities. Such MTL compounds could then be employed with a view to reducing dosage amounts. In particular dosage amounts of drugs in treatment of conditions of inflammation and pain may be reduced. To date, few such compounds have been identified.

Notwithstanding the prior art, therefore it is desirable to provide compounds that have balanced multi-target ligand actions, in particular those which can activate simultaneously, at least one of the PPARs and at least one of the cannabinoid¹⁰ receptors.

Dual functionality may be achieved by having ligands that are active on different receptors. In particular, it would be desirable to provide multitarget compounds which are active at, at least one of the PPAR-α,γ,δ (alpha, γ and delta) receptors (referred to in the following text as PPARs) and at least one cannabin, for example the CB1 or the CB2 receptor. It is further desirable to provide pharmaceutical compositions comprising such compounds for use in the medical field. It will be appreciated that such compounds will have ligands ideally with at least dual functionality. However, it is still desirable to have compounds with activity at a single receptor. Of particular interest in the present invention are compounds which are dual PPAR/cannabinoid agonists, pharmaceutical compositions containing them and their use in the medical field. Those skilled in the art will know that the PPAR-δ (delta) is often times referred to as PPAR-β (beta) and the two names are synonymous.

Emerging evidence supports the possibility that compounds able to act on both CB2 and PPAR-γ receptors may be of unprecedented therapeutic benefit in debilitating pathological conditions affecting the central nervous system (CNS), such as stroke, multiple sclerosis, Alzheimer's disease and other chronic neurodegenerative disorders. Thus such compounds are highly desirable.

According to the present invention, as set out in the appended claims, in a first aspect, there is provided a compound having activity at, at least one of a PPAR and a cannabinoid receptor, comprising a PPAR pharmacophore and a cannabinoid pharmacophore linked together by
(i) a moiety comprising a fused bicyclic ring; or
(ii) the cannabinoid pharmacophore comprising a fused bicyclic ring and the PPAR pharmacophore linked to the bicyclic ring of the cannabinoid pharmacophore;

the PPAR pharmacophore comprising a salicylic acid functionality, an alkoxybenzylacetic acid functionality or an alkoxyphenylacetic acid functionality.

The compounds of the invention also relate to the compounds described herein, a tautomer thereof, a pharmaceutically acceptable salt thereof, or a hydrate thereof.

In one embodiment, there is provided a compound having activity at both PPAR and cannabinoid receptors comprising a PPAR pharmacophore and a cannabinoid pharmacophore linked together by
(i) a moiety comprising a fused bicyclic ring; or
(ii) the cannabinoid pharmacophore comprising a fused bicyclic ring and the PPAR pharmacophore linked to the bicyclic ring of the cannabinoid pharmacophore;

the PPAR pharmacophore comprising a salicylic acid, alkoxybenzylacetic acid or a alkoxyphenylacetic acid functionality.

Preferably, the compounds of the invention show agonist activity at both a PPAR and a cannabinoid receptor. However in another aspect, the compounds may have activity at, at least one of a PPAR and cannabinoid receptor. In this particular aspect, particularly preferred are those compounds which have activity at a PPAR receptor. The most preferred compounds of this aspect have activity at a PPAR-γ receptor. Most preferable of all are those compounds which show agonist activity at a PPAR receptor, which is the PPAR-γ receptor.

In one embodiment, in which the compounds as described herein have such dual PPAR and cannabinoid receptor activity, the PPAR pharmacophore is linked to the fused bicyclic ring through an amine or an amide functional group.

In a second aspect, the compounds of the invention may comprise a fused bicyclic ring which forms part of the cannabinoid pharmacophore. Thus herein, the term cannabinoid pharmacophore includes a group that is bound to a fused bicyclic ring linker such that either the group itself or the group in combination with the ring system has the ability to activate the cannabinoid receptor of interest.

By this definition, it is intended to mean that the cannabinoid pharmacophore comprises a fused bicyclic ring falling under the definition provided earlier here.

Similarly, the term PPAR pharmacophore includes a group that is bound to a fused bicyclic ring linker such that either the group itself or the group in combination with the ring system has the ability to activate the PPAR of interest.

In a second aspect, the preferred compounds of the invention suitably comprise a fused bicylic ring which is part of the cannabinoid pharmacophore, with the proviso that the fused bicylic ring system which is part of a cannabinoid pharmacophore does not form part of a cannabinoid pharmacophore antagonist moiety.

Thus in a preferred embodiment, there is provided a compound having activity at, at least one of a PPAR and a cannabinoid receptor, wherein said compound comprises:

a cannabinoid pharmacophore comprising a fused bicyclic aromatic ring or partially aromatic ring; and
a PPAR pharmacophore comprising a salicylic acid functionality, an alkoxybenzylacetic acid functionality or a alkoxyphenylacetic acid functionality; and
wherein the PPAR pharmacophore is covalently bound to the cannabinoid pharmacophore through an amide or amine linkage; and a pharmaceutically acceptable salt thereof.

As used herein, the term “partially aromatic” may be taken to have the meaning that the bicyclic ring includes a benzo moiety fused to a non-aromatic ring or to a ring that is not completely unsaturated. A fused ring is a ring system wherein two rings are fused together which means two contiguous atoms are shared by and form part of each ring. Preferably, the bicyclic ring system comprises a fused 8-10 atom ring system.

In a preferred embodiment, there is provided a compound having activity at least one of a PPAR and a cannabinoid receptor comprising:

a PPAR pharmacophore and a cannabinoid pharmacophore linked together by a moiety comprising a fused bicyclic ring comprising a five membered ring fused with a six membered ring or a six membered ring fused with a six membered ring,

wherein the cannabinoid pharmacophore comprises the fused bicyclic ring; and

the PPAR pharmacophore comprises a salicylic acid functionality, an alkoxybenzylacetic acid functionality or a alkoxyphenylacetic acid functionality; and

wherein the PPAR pharmacophore is linked to the bicyclic ring of the cannabinoid pharmacophore through a linker comprising an amine or an amide functional group.

The term “acid functionality” covers simple carboxylic acids and carboxyl acid esters and corresponding bioisosteric groups such as thiocarbonyl and thicarbonyl esters of same. Salicylic acid functionalities include:

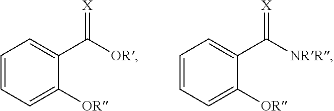

wherein X may be O or S, R′ and R″ may be independently selected from C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group. However, salicylamide type acid functionalities are least preferred, since the PPARs binding mode is expected to require an acidic or corresponding bioisosteric group.

Similarly, the alkoxybenzylacetic acid functionality or the alkoxyphenylacetic acid functionality may be represented by:

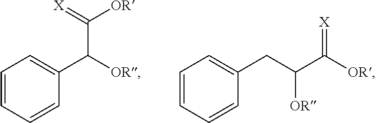

wherein X may be O or S, R′ and R″ may be independently selected from C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group.

Typically, PPAR pharmacophores are receptor binding portions comprising a salicylic acid or carboxylic acid and hydroxyl functionality such as those that are found in the group of compounds comprising glitazones-glitazars, 5-ASA, 4-ASA, 2-benzoylamino-benzoic acid, alpha-alkyloxyphenylproprionic acid, alpha-aryloxyphenylproprionic acid, salicylic acid, phthalic acid, or a compound comprising a thiazolidine cycle. Typically, PPAR pharmacophores are receptor binding portions comprising a salicylic acid, an alpha-alkyloxy- or aryloxy-phenylproprionic acid, a thiazolidine-2,4-dione cycle, a phthalic acid or a carboxylic acid such as those that are found in the group of compounds comprising 5-ASA, 4-ASA, glitazars, glitazones, di(2-ethylhexyl)phthalate (DEHP) or 2-benzoylamino-benzoic acid. However, the PPAR pharmacophores of the invention are preferably groups comprising a salicylic acid or carboxylic acid (—C(O)OH or acid esters of same) and hydroxyl functionality (—OH or esters)-OR of same).

In other preferred embodiments, the —OH of the salicylic acid group may be replaced by an alkoxy (—OR) substituent, wherein —OR is C₁-C₈alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group

In another embodiment, the compounds comprise the carboxylic acid ester analogues of the above PPAR acid functionalities, where the carboxylic acid functionality comprises an ester substituent which is a C₁-C₈alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group, substituted for the PPAR pharmacophore's carboxylic acid OH group. These compounds thus comprise a C₁-C₅alkoxyl (—OR^{alk}), a C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp) or benzyloxy (—OCH₂Ph) group substituent on the PPAR pharmacophore's carboxylic acid OH group. (—OR^{alk}(cyc)) represents an —OcyclicC₃-C₆ alkyl group.

Thus, the compounds of the invention may comprise also the carboxylic acid analogues of the compounds, where the ester substituent is a C₁-C₈alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group substituent on the PPAR pharmacophore's carboxylic acid OH group.

However, the most preferred PPAR pharmacophores of the compounds of the present invention are those having a salicylic acid functionality, an alkoxybenzylacetic acid functionality or an alkoxyphenylacetic acid functionality, including the carboxylic acid and carboxylic acid esters of same. However, PPAR pharmacophores comprising a salicylic acid group, an alkoxybenzylacetic acid or an alkoxyphenylacetic acid functionality are particularly preferred. Thus, the PPAR pharmacophore may be a simple salicylic acid functionality, an alkoxybenzylacetic acid functionality or a alkoxyphenylacetic acid functionality. In a preferred embodiment the acid functionality comprises a simple —C(O)OH acid group.

Thus, typically, the preferred PPAR pharmacophore of the invention comprises a moiety selected from the group consisting of:

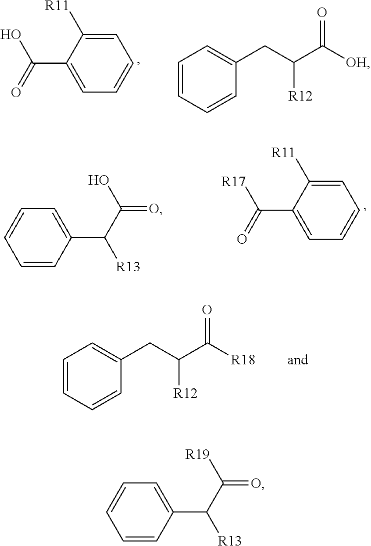

wherein R¹¹, R¹², and R¹³ are each independently selected from the group consisting of: OH, C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) and a phenylphenoxy (—OPhPh) group; and R¹⁷, R¹⁸ and R¹⁹ are each independently selected from the group consisting of: OH, C₁-C₈alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) and a phenylphenoxy (—OPhPh) group.

Thus, typically, the preferred PPAR pharmacophore of the invention comprises a moiety selected from the group consisting of:

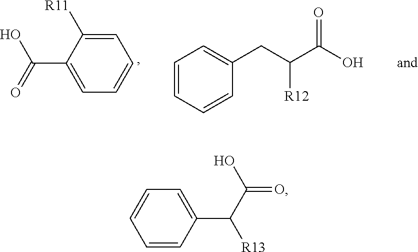

wherein R₁₁, R₁₂, and R₁₃ are each independently selected from the group consisting of: OH, C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) and a phenylphenoxy (—OPhPh) group.

The compounds of the invention contain a PPAR pharmacophore that herein is taken to be a chemical functionality that comprises a salicylic acid, an alkoxybenzylacetic acid or an alkoxyphenylacetic acid functionality or derivatives of same. For example, the alkoxybenzylacetic acid or alkoxyphenylacetic acid functionalities can be substituted at the carboxyl OH with groups such as C₁-C₅ alkoxyl or C₃-C₆ cycloalkoxyl groups. Particularly preferred are groups such as C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or phenylphenoxy (—OPhPh) group substituents in place of —OH. The acid functionality comprises a salicylic acid functionality, an alkoxybenzylacetic acid functionality or an alkoxyphenylacetic acid functionality having a —C(O)OH carboxylic acid group and derivatives of same, i.e. acid esters (—C(O)OR). Alkenoxyl group substituents, such as C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group can also be used in place of the —OH group.

The PPAR pharmacophore functionalities also include for the alkoxybenzylacetic acid functionality or the alkoxyphenylacetic acid functionalities, derivates where the —C(O)OH remains intact and the alkoxyl group can be groups such as C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group. Furthermore, for the alkoxybenzylacetic acid functionality or the alkoxyphenylacetic acid functionality, the PPAR pharmacophores of the invention may comprise carboxylic acid ester derivates of the acid functionality where the acid ester groups include alkenoxyl group substituents, such as C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group can also be used. However, PPAR pharmacophores comprising a simple salicylic acid group, an alkoxybenzylacetic acid or an alkoxyphenylacetic acid functionality are particularly preferred.

Suitably, the amine or an amide functional group linker can be any group comprising an amine or an amide functionality.

Typically, preferred amine/or amide linkers can be selected from the group consisting of —X′NR′—, —NR′—, —C(O)NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—,

in which R′ and R″ are independently hydrogen, optionally substituted C₁-C₈ alkyl, C₃-C₁₀ cycloalkyl, aryl, heteroaryl, aralkyl, alkoxy or heteroaralkyl; and

X′ and X″ are independently a bond, —NH—, piperzine, C₁-C₈ alkyl, a C₁-C₈ alkylene or C₁-C₈ alkyl.

In particularly preferred embodiments, the amine or amide linker can be selected from the group consisting of: —X′NR′—, —NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—, in which R′ is hydrogen, optionally substituted C₁-C₈ alkyl, C₃-C₁₀ cycloalkyl, aryl, heteroaryl, aralkyl, alkoxy or heteroaralkyl; and X′ and X″ is independently a bond, —NH—, piperzine, C₁-C₈ allyl, a C₁-C₈ alkylene or C₁-C₈ alkyl; R″ is optionally substituted C₁-C₈ alkyl, C₃-C₁₀ cycloalkyl, aryl, heteroaryl, aralkyl, alkoxy or heteroaralkyl;

However, in a particularly preferred embodiment, the amine or amide linker can be selected from the group consisting of —CH₂NH—, —NH—, —C(O)NHNH—, —C(O)NC₂H₄N— and —C(O)NHCH₂CH₂—.

In the most preferred embodiments the amide linker is selected from the group consisting of —C(O)NHNH—, —C(O)NC₂H₄N— and —C(O)NHCH₂CH₂—.

Suitably, in an embodiment comprising an amide linker, it is preferred that the carbonyl group of the amide linker is located in a position closest to the fused ring system. This arrangement advantageously provides a H-bond interaction point with the receptor in the putative binding site of the receptor model used herein.

The PPAR pharmacophore may link to the amine or amide linker at any one of the phenyl ring positions. However, the most preferred PPAR pharmacophores for the compounds of the invention can be selected from the group comprising

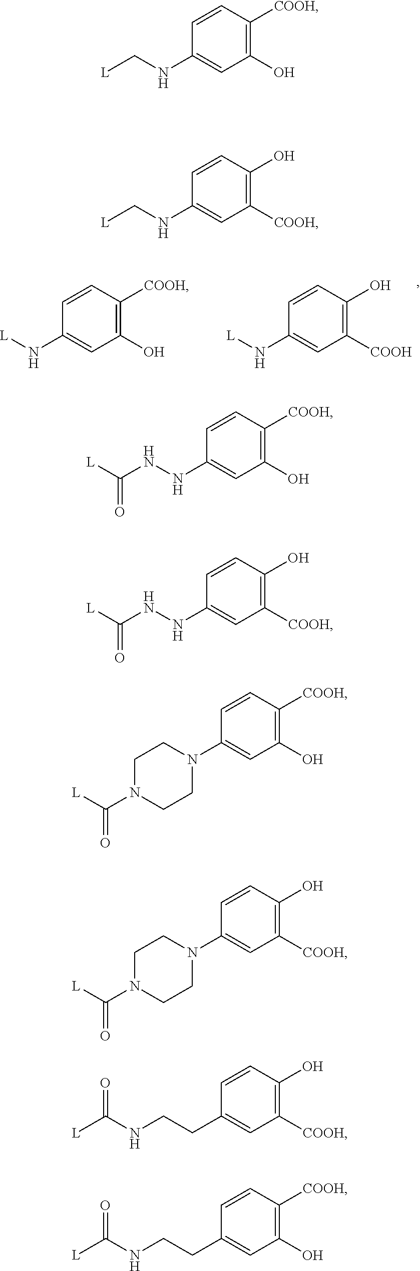

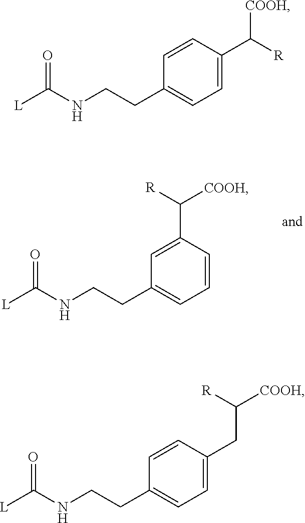

wherein L represents the amine or amide linker.

With reference to the second aspect, preferred PPAR pharmacophores for the compounds of the invention can be selected from the group comprising

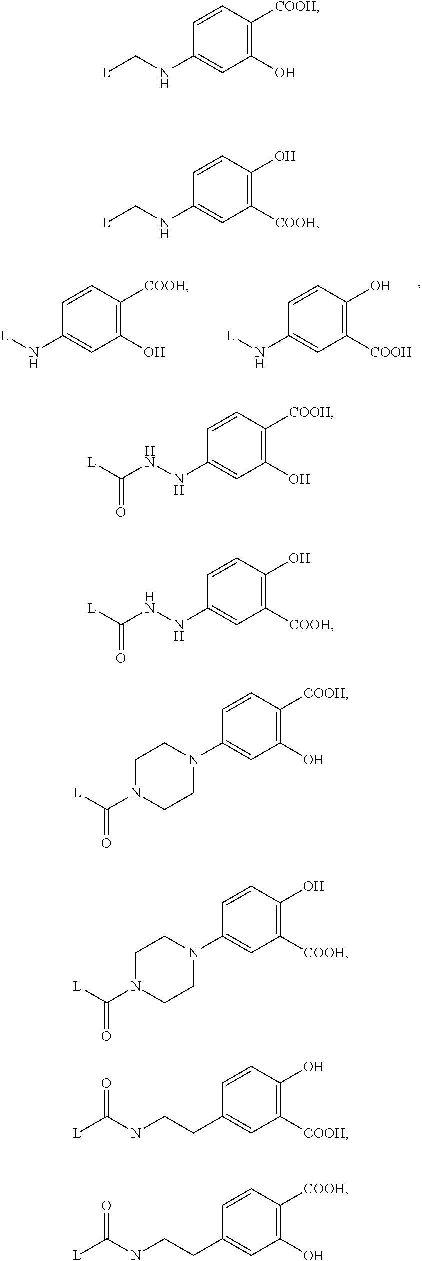

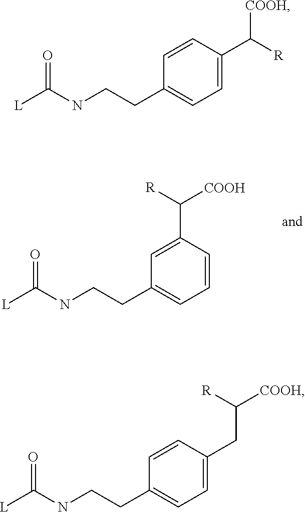

wherein L is the fused bicyclic ring to which the PPAR pharmacophore is attached and R is H, a C₁-C₅ alkoxyl, a C₃-C₆ cycloalkoxyl group, a vinyloxyl, a C₃-C₅ allyloxyl, benzoxy, naphthaloxy or a benzyloxy group. C₁-C₈ alkoxyl may also be suitably used.

In an embodiment comprising an amine group as defined above and wherein X′ or X″ is a bond, it is preferred that the nitrogen of the amine group is directly linked to the phenyl group of the salicylic acid, the alkoxybenzylacetic acid or the alkoxyphenylacetic acid functionality.

In the second aspect of the invention, these representative structures show the PPAR pharmacophores of the invention linked to the most preferable amine or amide linkers, wherein L represents the linkage to the fused bicyclic cannabinoid pharmacophore to which the PPAR pharmacophore is attached, and wherein —R can be H to provide —OH, or R can be —OR to provide alkoxy groups, wherein —OR is a C₁-C₈ alkoxy, C₃-C₆ cycloalkoxyl (—OR^{alk}(cyc), group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group.

Thus, particularly preferred compounds are those wherein the amide or amine linkage is covalently bound to the PPAR pharmacophore and is selected from the group consisting of:

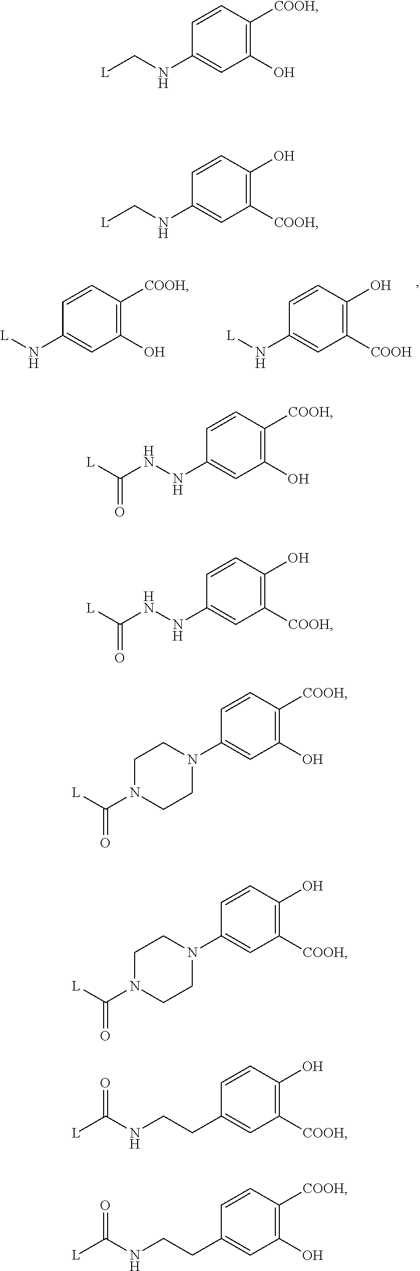

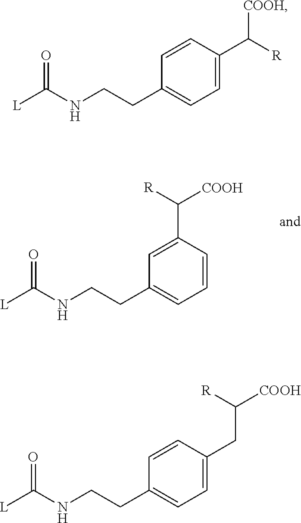

wherein L represents the fused 8-10 membered cannabinoid pharmacophore bicyclic aromatic or partially aromatic ring; and R is selected from the group consisting of C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) and a phenylphenoxy (—OPhPh) group.

PPAR pharmacophores joined to the fused cannabinoid pharmacophore ring of the second aspect of the invention through an amide linker wherein the carbonyl of the amide linker is directly attached to the fused bicyclic ring are particularly desirable, since a carbonyl group joined to the fused ring advantageously provides a H-bond interaction point with the receptor in the putative binding site of the receptor model used herein. Thus compounds wherein the PPAR pharmacophore is linked to the fused ring through the carbonyl of an amide group are particularly preferred. Thus particularly preferred PPAR pharmacophore and amide linkers may be selected from the group consisting of:

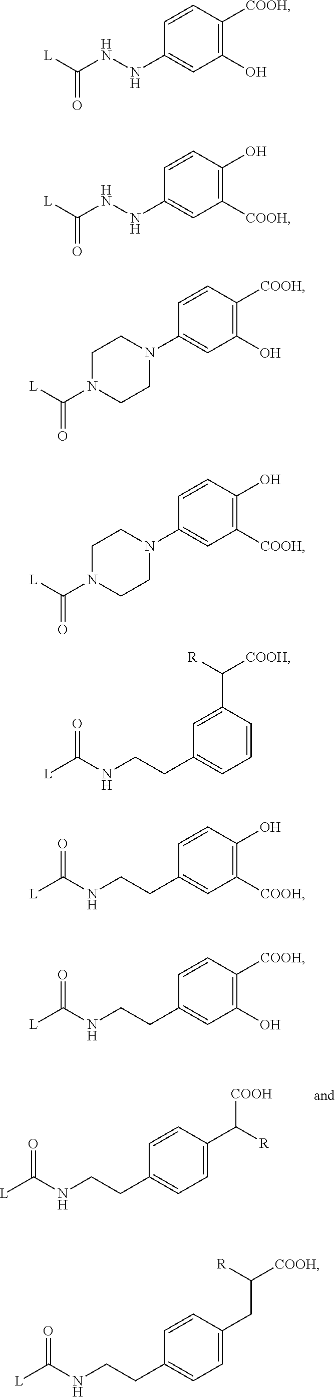

Compounds of the invention comprising these particular PPAR pharmacophores together with an amide linker are particularly preferred, when the fused bicylic ring of the cannabinoid pharmacophores does not have another carbonyl containing substituent attached thereto.

For the sake of clarity with regard to the present invention, the inventor does not wish to set out a strict pharmacological definition of what molecular functionalities constitute cannabinoid pharmacophores or PPAR pharmacophores.

There are many chemical functional groups or systems that are reported to bind to cannabinoid receptors. Typical examples of such chemical entities are classical THC type structure, aminoalkylindoles, eicosanoids related to the endocannabinoids, 1,5-diarylpyrazoles and quinolines. With the exception of the eicosanoids, many of these compounds contain fused cyclic ring systems which may or may not play a role in receptor binding. Unfortunately, it is not always clear-cut which functional groups bind to the cannabinoid receptors. In other words, there is no clear unanimous picture of what the typical cannabinoid pharmacophore precisely is. The diversity of the structure of the known cannabinoid active molecules highlights this point. Good starting points for cannabinoid pharmacophores may be found in AJA, WIN-55212-2 and JTE907 compounds. Many cannabinoid systems are known to contain fused cyclic ring systems and particularly ring systems having a tricyclic fused ring system, which may or may not play a role in receptor binding.

The compounds of the invention have a fused bicyclic ring, which comprises two rings selected from the group comprising thiophenes, [1,2,5]-thiadiazolines, pyrroles, imidazoles, thiazoles, pyrazoles, 4,5-dihydropyrroles, imidazolidin-2-ones, 1,2,3,4-tetrahydro-pyrazines, benzenes, pyridazines, pyridines, pyrimidines, pyrazines, 4,5-dihydrothiophenes and imidazolidin-2-thiones. Thus each ring of the fused bicyclic aromatic or partially aromatic ring may be independently selected from the group consisting of thiophene, [1,2,5]-thiadiazoline, pyrrole, imidazole, thiazole, pyrazole, 4,5-dihydropyrrole, imidazolidin-2-one, 1,2,3,4-tetrahydro-pyrazine, benzenes, pyridazine, pyridine, pyrimidine, pyrazine, 4,5-dihydrothiophene and imidazolidin-2-thione.

The fused rings may comprise carbon atoms only or may comprise at least one heteroatom substituted for a carbon of the fused ring. Typically, rings such as the following may form part of the fused bicyclic ring system

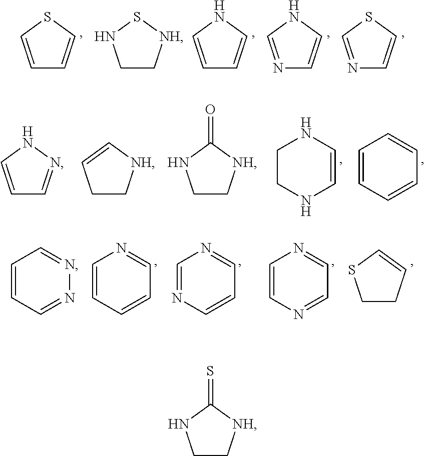

wherein the fused bicyclic ring comprises a five membered ring fused with a six membered ring or a six membered ring fused with a six membered ring.

In a preferred embodiment, suitably, the fused ring system comprises a benzene, pyrrole or a pyridine ring.

A variety of ring combinations may be selected as the fused bicyclic linker and the rings may be fused together in a number of ways to produce many different fused ring systems.

However, in a preferred embodiment, the fused bicyclic ring comprises a benzo fused pyrrole, a benzo fused pydridine, a benzo fused thiophene, a benzo fused imidazole, a benzo fused thiazole, a benzo fused [1,2,5]-thiadiazoline, a benzo fused pyrazole, a benzo fused 4,5-dihydropyrrole, a benzo fused imidazolidin-2-one, a benzo fused 1,2,3,4-tetrahydro-pyrazine, a benzo fused benzene, a benzo fused pyridazine, a benzo fused pyridine, a benzo fused pyrimidine, a benzo fused pyrazine, a benzo fused 4,5-dihydrothiophene or a benzo fused imidazolidin-2-thione.

Thus, the fused 8-10 member bicyclic aromatic or partially aromatic rings of the invention may be selected from the group consisting of: benzo fused pyrrole, benzo fused pydridine, benzo fused thiophene, benzo fused imidazole, benzo fused thiazole, benzo fused [1,2,5]-thiadiazoline, benzo fused pyrazole, benzo fused 4,5-dihydropyrrole, benzo fused imidazolidin-2-one, benzo fused 1,2,3,4-tetrahydro-pyrazine, benzo fused benzene, benzo fused pyridazine, benzo fused pyridine, benzo fused pyrimidine, benzo fused pyrazine, benzo fused 4,5-dihydrothiophene and benzo fused imidazolidin-2-thione.

In a particular embodiment the cannabinoid pharmacophore comprises a fused bicyclic ring selected from the group consisting of:

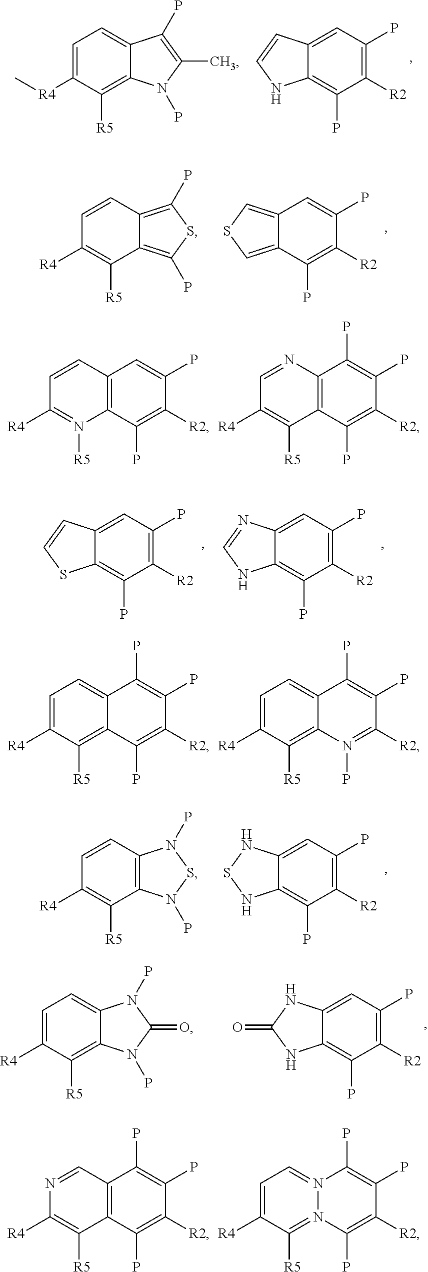

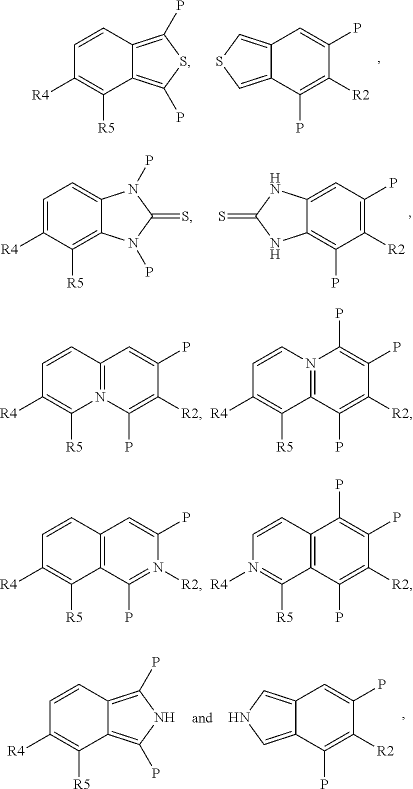

wherein
at least one P is H, a PPAR pharmacophore or a CB pharmacophore; R₁ is H; or forms part of a pharmacophore having activity at a PPAR or a cannabinoid receptor;
R₂ is H, methyl, ═O, ═S, ═NH, C₁-C₅ alkyl, C₁-C₅ alkoxy or a lone pair of electrons;
R₄ is H, methyl, ═O, ═S or NH, C₁-C₅ alkyl or C₁-C₅ alkoxy;
R₅ is H, methyl, ═O, ═S or NH, C₁-C₅ alkyl or C₁-C₅ alkoxy; and
   pharmacophore wherein the PPAR pharmacophore is linked to the bicyclic ring of the cannabinoid pharmacophore through a linker comprising an amine or an amide functional group.

In the second aspect of the invention, P can be a cannabinoid pharmacophore substituent. In such an embodiment, it is preferable that at least one of either of the PPAR pharmacophore and the cannabinoid pharmacophore substituent groups comprise a carbonyl group which is attached directly to the cannabinoid pharmacophore fused bicyclic ring.

In a preferred embodiment, the cannabinoid pharmacophore comprises a fused bicyclic ring selected from the group consisting of:

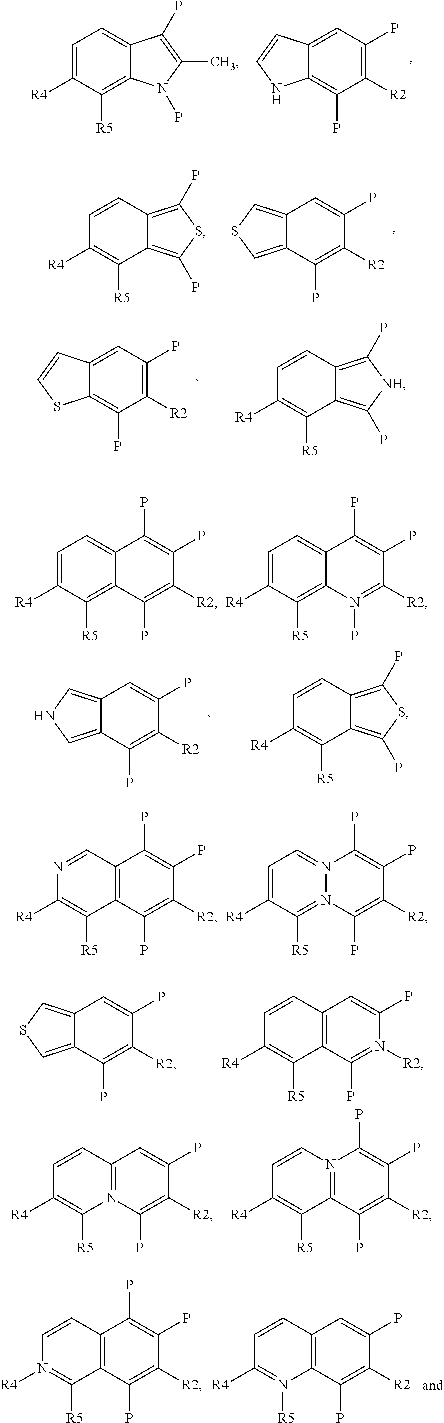

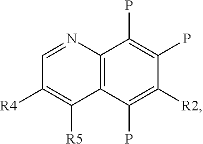

wherein
at least one P is H, a PPAR pharmacophore or a CB pharmacophore; R₁ is H; or forms part of a pharmacophore having activity at a PPAR or a cannabinoid receptor;
R₂ is H, methyl, ═0, ═S, ═NH, C₁-C₅ alkyl, C₁-C₅ alkoxy or a lone pair of electrons;
R₄ is H, methyl, ═O, ═S or NH, C₁-C₅ alkyl or C₁-C₅ alkoxy;
R₅ is H, methyl, ═O, ═S or NH, C₁-C₅ alkyl or C₁-C₅ alkoxy; and

pharmacophore wherein the PPAR pharmacophore is linked to the bicyclic ring of the cannabinoid pharmacophore through a linker comprising an amine or an amide functional group with the proviso that the fused bicylic rings which are part of a cannabinoid pharmacophore are not part of a cannabinoid antagonist moiety.

In such embodiments wherein the pharmacophores are positioned on a six membered ring, they may be positioned in a meta or a para arrangement to each other.

In particular embodiments, the compounds of the invention have a fused bicylic ring which can be substituted or unsubstituted atoms or groups such as H, methyl, ═O, ═S, or ═NH at the ring positions other than those of R₁, R₃ and R₆.

In preferred compounds comprising a fused 8-10 member bicyclic aromatic or partially aromatic ring, the ring system may be optionally substituted by one, two or three substituents each independently selected from C₁-C₈ alkyl, ═O, ═S, ═NH, or C₁-C₈alkoxy, at a position other than R₁, R₂ or R₃.

In some embodiments, the fused bicyclic ring can be selected from the following group:

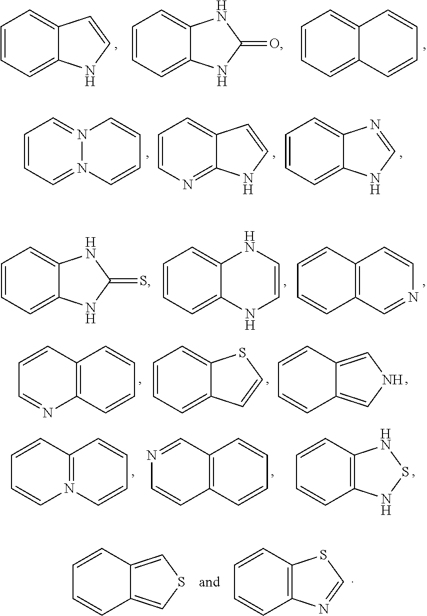

The preferred compounds of the invention comprise fused bicylic rings which form part of the cannabinoid pharmacophore with the proviso that the cannabinoid pharmacophore in question is not a cannabinoid antagonist or part of a cannabinoid active molecule which has antagonist activity.

In a preferred embodiment of the invention the fused bicyclic ring does not comprise oxygen as a ring heteroatom. However, suitably, at least one ═O group (exocyclic O) can be positioned as a bicylic ring substituent.

However, in a preferred embodiment the bicyclic ring system consists of two fused rings wherein at least one heteroatom is N or S.

In a particularly preferred embodiment the fused bicyclic ring of the invention comprises carbon atoms only or a single N heteroatom positioned in the fused ring system in place of a carbon atom.

However, in a particularly preferred embodiment, the fused bicylic ring comprises a benzo-fused pyrrole or a benzo-fused pyridine ring system.

In another preferred embodiment, both of the rings of the fused bicyclic ring system are aromatic.

It is however, particularly preferred that the compounds of the invention comprise a bicyclic ring selected from the group consisting of:

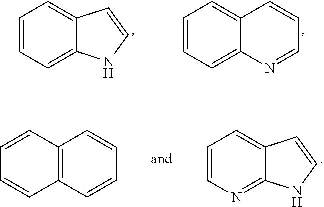

The benzo fused-pyrrole or a benzo-fused pyridine ring systems are particularly preferred. Thus cannabinoid pharmacophores having these particular types of ring system are highly desirable.

In an embodiment, where the compounds comprise a quinoline ring as the fused bicyclic ring, it is desirable to have a ═O (exocyclic O) group positioned on the heterocyclic ring at the ring atom located between R₁ and R₃.

It is more particularly preferred in these cases to have alkoxy substituents on the non-heterocyclic ring of the quinoline bicyclic. Suitable alkoxy substituents include C₁-C₁₀ alkyl alkoxide groups, however disubstituted rings having a C₁ to C₅ alkyl alkoxide group are most particularly preferred.

It is particularly preferred in this embodiment to have at least one alkoxy substituent on the non-heterocyclic ring of the quinoline bicyclic system. Suitable alkoxy substituents include C₁-C₁₀ alkylalkoxide groups. The most favourable compounds comprise disubstituted rings, wherein the quinoline substituted with two C₁ to C₅ alkylalkoxide groups.

In the first aspect of the invention, wherein the cannabinoid and the PPAR pharmacophores linked by a linker having a fused bicyclic ring portion, typical suitable cannabinoid pharmacophores can be considered as functional groups which comprise a carbonyl moiety bound to an alkyl, cycloalkyl, or aromatic ring such as a benzene or a naphthylene ring and ring derivates of same. Attachment to the fused bicyclic linker occurs at the carbonyl group. This is an advantageous arrangement, since carbonyl joined to the fused ring advantageously provides a H-bond interaction point with the receptor in the putative binding site of the receptor model used herein.

Thus in this first aspect, arylcarboxy, cycloalkylcarboxy, alkylcarboxy, arylcarbamoyl, cycloalkylcarbamoyl or alkylcarbamoyl groups can be used as cannabinoid pharamacophore substituents falling within the meaning of term “cannabinoid pharmacophore” as described herein. Preferably the aryl group of the above mentioned cannabinoid substituents may include arylalkoxy or arylhalide derivates thereof. Cannabinoid substituents having a carbonyl group disposed therein next to the fused ring are advantageous arrangements, since carbonyl joined to the cannabinoid pharmacophore fused ring advantageously provides a H-bond interaction point with the receptor in the putative binding site of the receptor model used herein. Suitably, an arylcarboxy, C₁-C₈ cycloalkylcarboxy, C₁-C₅ alkylcarboxy, arylcarbamoyl, C₁-C₈ cycloalkylcarbamoyl, C₁-C₅ alkylcarbamoyl groups can also suitably be used as cannabinoid pharamacophores substituents falling within the meaning of the term as described herein. Preferable aryl group derivates include arylalkoxy or arylhalide derivates,

wherein L represents the fused bicyclic linker to which the cannabinoid pharmacophore is bound.

An alternative simpler functional group comprises alkyl chains that can be straight-chained or branched.

Thus in this first aspect, preferred cannabinoid pharmacophores of the invention can be selected from the group comprising:

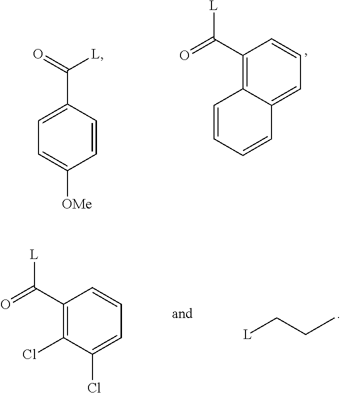

Particularly preferred compounds of the invention comprise a cannabinoid pharmacophore which may be:

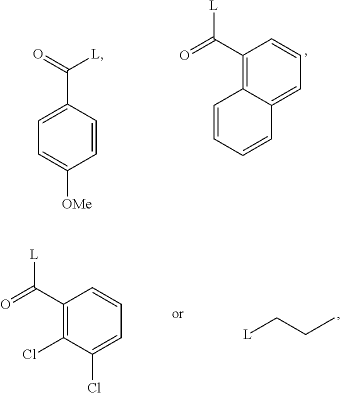

wherein L represents the fused 8-10 member bicyclic aromatic or partially aromatic ring.

The at least one group substitution may be independently positioned on the same or different rings of the fused bicyclic system.

In embodiments of the second aspect of the invention, where the fused ring is part of the cannabinoid pharmacophore, typical suitable cannabinoid pharmacophores bicyclic ring substituents can be considered as functional groups which comprise a carbonyl moiety bound to an alkyl, cycloalkyl, or aromatic ring such as a benzene or a naphthylene ring and ring derivates of same. Attachment to the fused bicyclic linker occurs at the carbonyl group. This is an advantageous arrangement, since carbonyl joined to the fused ring advantageously provides a H-bond interaction point with the receptor in the putative binding site of the receptor model used herein.

Suitably, arylcarbamoyl, cycloalkylcarbamoyl or alkylcarbamoyl groups can also be suitably used as cannabinoid pharamacophores substituents falling within the meaning of term as described herein. Thus in the second aspect, wherein the fused bicyclic ring forms part of a cannabinoid pharmacophore, preferred cannabinoid pharmacophores substituents of the invention can be selected from the group consisting of:

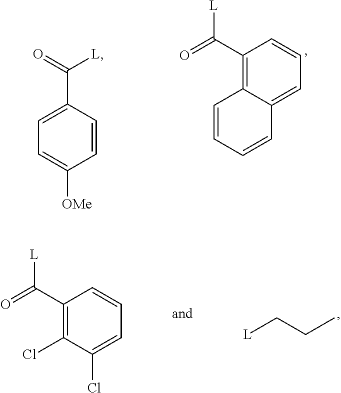

wherein L represents the fused bicyclic linker to which the cannabinoid pharmacophore is bound. Preferably the aryl group derivates of the above mentioned cannabinoid pharmacophore derivates include arylalkoxy or arylhalide derivates thereof. Groups having carbonyl substituents joined to the fused ring system are advantageous arrangements, since carbonyl joined to the fused ring advantageously provides a H-bond interaction point with the receptor in the putative binding site of the receptor model used herein.

Thus, in one embodiment relating to the second aspect of the invention, the preferred compounds of the invention comprise a PPAR pharmacophore comprising an amine linker which is selected from the group consisting of:

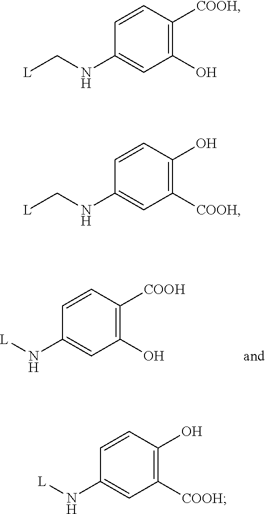

and wherein the cannabinoid fused bicylic ring further comprises a substituent selected from the group consisting of:

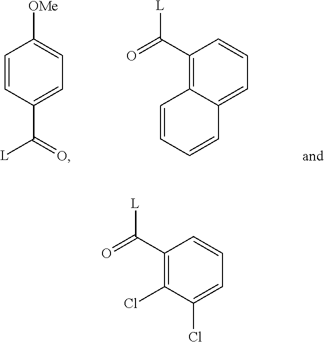

wherein L represents the fused bicycle ring to which the cannabinoid substituent and the PPAR pharmacophore (plus linker) is attached. This ensures that the compounds have the carbonyl substituent joined to the fused ring provide the H-bond interaction point with the receptor, preferred in the putative binding site of the receptor model used herein.

In another preferred embodiment, there is provided a compound having activity at, at least one of a PPAR and a cannabinoid receptor comprising, wherein said compound comprises:

a cannabinoid pharmacophore comprising a fused bicyclic ring; and

a PPAR pharmacophore comprising a moiety selected from the group consisting of:

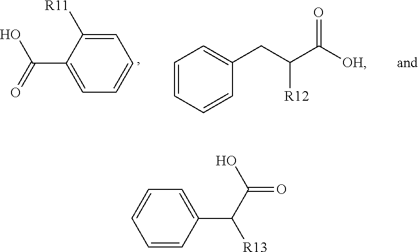

wherein:

R₁₁, R₁₂ and R₁₃ are each independently selected from the group consisting of: OH, C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) and a phenylphenoxy (—OPhPh) group; and

wherein the PPAR pharmacophore is covalently bound to the cannabinoid pharmacophore through an amide or amine linkage; and a pharmaceutically acceptable salt thereof.

Preferred compounds of the invention comprise:

a cannabinoid pharmacophore comprising a fused 8-10 member bicyclic aromatic or partially aromatic ring; and

a PPAR pharmacophore comprising a moiety selected from the group consisting of:

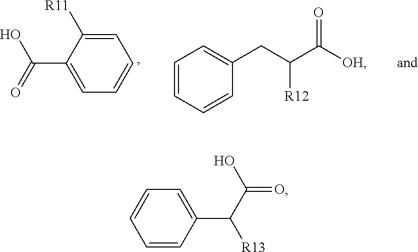

wherein:
R₁₁, R₁₂, and R₁₃ are each independently selected from the group consisting of: OH, C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) and a phenylphenoxy (—OPhPh) group; and wherein the PPAR pharmacophore is covalently bound to the cannabinoid pharmacophore through an amide or amine linkage; and a pharmaceutically acceptable salt thereof.

Relating to the first aspect, in particular embodiments, the compounds of the invention have the general structure (I)

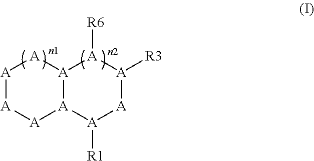

wherein
n is 0 or 1;
A represents an atom of the fused bicyclic ring;
R₁ is H or is part of the pharmacophore having activity at a PPAR or a cannabinoid receptor; either one of R₃ or R₆ is H or is part of the pharmacophore having activity at a PPAR or a cannabinoid receptor;
   wherein the PPAR pharmacophore comprises a salicylic acid, an alkoxybenzylacetic acid, or an alkoxyphenylacetic acid functionality.

In such embodiments wherein the pharmacophores are positioned on a six membered ring, they may be positioned in a meta or a para arrangement to each other.

In a particularly preferred embodiment related to the second aspect of the invention, the compounds of the invention have the general structure (I)

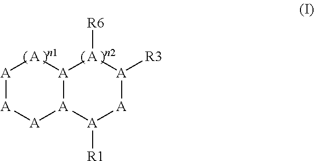

wherein

n is 0 or 1;

A represents an atom of the fused bicyclic ring of the cannabinoid pharmacophore;

R₁ is H or is part of the pharmacophore having activity at a PPAR receptor or is a cannabinoid pharmacophore substituent;

either one of R₃ or R₆ is H or is part of the pharmacophore having activity at a PPAR receptor or is a cannabinoid pharmacophore substituent;

wherein the cannabinoid pharmacophore comprises the fused bicyclic ring; and
wherein the PPAR pharmacophore comprises a salicylic acid, an alkoxybenzylacetic acid or an alkoxyphenylacetic acid functionality; and

the PPAR pharmacophore is linked to the bicyclic ring of the cannabinoid pharmacophore through a linker comprising an amine or an amide functional group.

In particular embodiments the PPAR pharmacophore carboxylic acid OH group can be substituted with a C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) and a phenylphenoxy (—OPhPh) group. This means that the —OH of —C(O)OH group or the —OH of the salicylic acid group may be substituted with an alkoxy group such as C₁-C₅ alkoxyl, a C₃-C₆ cycloalkoxyl group, a vinyloxyl, a C₃-C₅ allyloxyl, benzoxy, naphthaloxy or a benzyloxy group.

The alkoxy groups of the alkoxybenzylacetic acid or a alkoxyphenylacetic acid functionality may also comprise an alkoxy group such as C₁-C₅ alkoxyl, a C₃-C₆ cycloalkoxyl group, a vinyloxyl, a C₃-C₅ allyloxyl, benzoxy, naphthaloxy or a benzyloxy group. The acid functionality may be —C(O)OH or carboxylic acid esters of same or equivalent bioisoteric groups and derivates.

However, Z comprising a salicylic acid functionality, an alkoxybenzylacetic acid functionality or an alkoxyphenylacetic acid functionality is particularly preferred.

In some embodiments Z further comprises a substitution at the PPAR pharmacophore carboxylic acid OH group, wherein the OH is substituted with a C₁-C₅ alkoxyl, a C₃-C₆ cycloalkoxyl group, a vinyloxyl, a C₃-C₅ allyloxyl, benzoxy, naphthaloxy or benzyloxy group.

Suitably, an arylcarboxy, C₁-C₈ cycloalkylcarboxy, C₁-C₅ alkylcarboxy, arylcarbamoyl, C₁-C₈ cycloalkylcarbamoyl, C₁-C₅ alkylcarbamoyl groups can also suitably be used as cannabinoid pharamacophores substituents falling within the meaning of term as described herein. Preferable aryl group derivates include arylalkoxy or arylhalide derivates. Preferably, the cannabinoid pharmacophore substituent may be selected from the group consisting of:

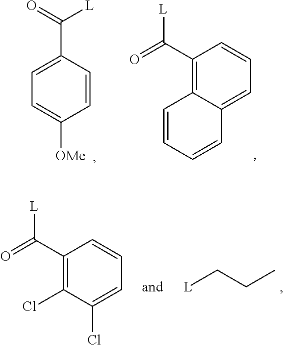

wherein L represents the fused bicyclic linker to which the cannabinoid pharmacophore is bound.

In embodiments wherein the cannabinoid pharmacophore substituent is:

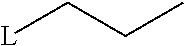

it is preferred that the linker between the fused ring of the cannabinoid pharmacophore and the PPAR pharmacophore is an amide group linker, wherein the carbonyl of the amide group is located directly next to the fused ring.

Typically, preferred amine or amide linkers can be selected from the group consisting of —X′NR′—, —NR′—, —C(O)NR′—, —C(O)NR′R″—, —NR′C(O)R″—, —C(O)NR′NR″—, —X′NR″R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—,

in which R′ and R″ are independently hydrogen, optionally substituted C₁-C₈ alkyl, C₃-C₁₀ cycloalkyl, aryl, heteroaryl, aralkyl, alkoxy or heteroaralkyl; and

X′ and X″ is independently a bond, —NH—, piperzine, C₁-C₈ alkyl, a C₁-C₈ alkylene or C₁-C₈ alkyl.

In particularly preferred embodiments, the amine or amide linker can be selected from the group consisting of: —X′NR′—, —NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—, in which R′ is hydrogen, optionally substituted C₁-C₈ alkyl, C₃-C₁₀ cycloalkyl, aryl, heteroaryl, aralkyl, alkoxy or heteroaralkyl; and X′ and X″ is independently a bond, —NH—, piperzine, C₁-C₈ alkyl, a C₁-C₈ alkylene or C₁-C₈ alkyl; R″ is optionally substituted C₁-C₈ alkyl, C₃-C₁₀ cycloalkyl, aryl, heteroaryl, aralkyl, alkoxy or heteroaralkyl;

However, in a particularly preferred embodiment, the amine or amide linker can be selected from the group consisting of —CH₂NH—, —NH—, —C(O)NHNH—, —C(O)NC₂H₄N— and —C(O)NHCH₂CH₂—.

In the most preferred embodiments the amide linker is selected from the group consisting of —C(O)NHNH—, —C(O)NC₂H₄N— and —C(O)NHCH₂CH₂—.

Other preferred compounds related to the second aspect have the general structure (I)

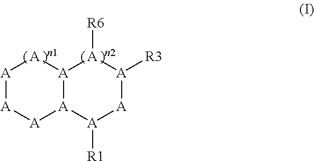

wherein
n¹ is 0 or 1;
n² is 0 or 1;

A represents an atom of the fused bicyclic ring of the cannabinoid pharmacophore;

R₁ is H or is part of the pharmacophore having activity at a PPAR receptor or is a cannabinoid pharmacophore substituent;

either one of R₃ or R₆ is H or is part of the pharmacophore having activity at a PPAR receptor or is a cannabinoid pharmacophore substituent;

wherein the cannabinoid pharmacophore comprises the fused bicyclic ring; and

wherein the PPAR pharmacophore comprises a salicylic acid, alkoxybenzylacetic acid or a alkoxyphenylacetic acid functionality; and

the PPAR pharmacophore is linked to the bicyclic ring of the cannabinoid pharmacophore through a linker comprising an amine or an amide functional group.

A preferred series of compound of the invention are represented by the general structure (I)

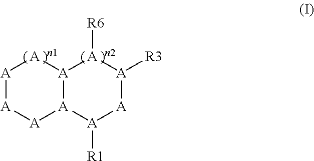

wherein
n¹ is 0 or 1;
n² is 0 or 1;
A represents an atom of the fused 8-10 member bicyclic aromatic or partially aromatic ring cannabinoid pharmacophore;
one of R₁, R₃ or R₆ is R₁₄, wherein R₁₄ is the amide or amine linkage covalently bound to the PPAR pharmacophore;
R₁ is selected from H, C₁-C₈alkyl or a cannabinoid pharmacophore comprising arylcarboxy, cycloalkylcarboxy, alkylcarboxy, arylcarbamoyl, cycloalkylcarbamoyl, alkylcarbamoyl or R₁₄;
R₃ is H, R₁₄, or is a cannabinoid pharmacophore substituent; and
R₆ is H, R₁₄, or is a cannabinoid pharmacophore substituent,

wherein cannabinoid pharmacophore substituent comprises an arylcarboxy, cycloalkylcarboxy, alkylcarboxy, arylcarbamoyl, cycloalkylcarbamoyl or alkylcarbamoyl group

Suitably, an arylcarboxy, C₁-C₈ cycloalkylcarboxy, C₁-C₅ alkylcarboxy, arylcarbamoyl, C₁-C₈ cycloalkylcarbamoyl, C₁-C₅ alkylcarbamoyl groups can also suitably be used as cannabinoid pharamacophores substituents falling within the meaning of term as described herein. Preferable aryl group derivates include arylalkoxy or arylhalide derivates. Preferably, the cannabinoid pharmacophore substituent may be selected from the group consisting of:

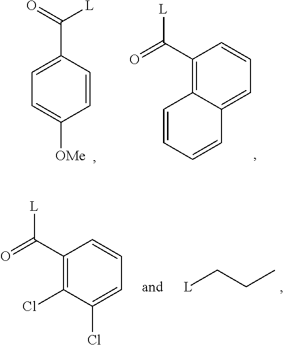

wherein L represents the fused bicyclic linker to which the cannabinoid pharmacophore is bound.

In particular embodiments relating to the first aspect, the compounds of the invention can be represented by the general formula (II) having activity at both PPAR and cannabinoid receptors

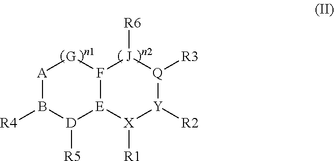

wherein
at least one of the rings is aromatic; at least one of n1 or n2 is 0 or 1; and

provided that at least one ring is aromatic,
A is CH, N or S; B is C, N or S; D is C or N; E is C or N; F is C or N; G is CH, N or S; X is C or N; Y is C, N or S; Q is C or N; J is CH, N or S; or
   provided that at least one ring is not aromatic,
A is CH, N, NH or S; B is C, N or S; D is C, N or S; E is C or N; F is C or N; G is CH, N, NH or S; X is C or N; Y is C, N or S; Q is C or N; J is CH, N or NH;
   and

R₁ is H or is part of a pharmacophore having activity at a PPAR or a cannabinoid receptor;

R₂ is H, methyl, ═O, ═S, ═NH or a lone pair of electrons;

R₃ is H; or forms part of a pharmacophore having activity at a PPAR or a cannabinoid receptor;

R₄ is H, methyl, ═O, ═S, ═NH, C₁-C₅ alkyl or C₁-C₅ alkoxy;

R₅ is H, methyl, ═O, ═S, ═NH, C₁-C₅ alkyl or C₁-C₅ alkoxy; and

R₆ is H; or forms part of a pharmacophore having activity at a PPAR or a cannabinoid receptor; with the proviso that
when B is S, R₄ is a lone pair of electrons; and
   with the added proviso that
when R₁ forms part of a pharmacophore having activity at a PPAR then R₃ forms part of a pharmacophore having activity at a cannabinoid receptor and when R₃ forms part of a pharmacophore having activity at a PPAR then R₁ forms part of a pharmacophore having activity at a cannabinoid receptor,
   wherein the PPAR pharmacophore comprises a salicylic acid, an alkoxybenzylacetic acid, or an alkoxyphenylacetic acid functionality.

In particular embodiments relating to the second aspect, the compounds of the invention can be represented by the general formula (II) having activity at, at least one of a PPAR and a cannabinoid receptor

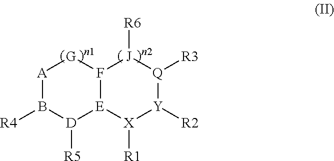

wherein

at least one of the rings is aromatic; at least one of n1 or n2 is 0 or 1; and
provided that at least one ring is aromatic,
   A is CH, N or S; B is C, N or S; D is C or N; E is C or N; F is C or N; G is CH, N or S; X is C or N; Y is C, N or S; Q is C or N; J is CH, N or S; or
provided that at least one ring is not aromatic,
   A is CH, N, NH or S; B is C, N or S; D is C, N or S; E is C or N; F is C or N; G is CH, N, NH or S; X is C or N; Y is C, N or S; Q is C or N; J is CH, N or NH;
      and
R₁ is H or is part of a pharmacophore having activity at a PPAR or is a cannabinoid pharmacophore substituent;
R₂ is H, methyl, ═O, ═S, ═NH or a lone pair of electrons;
R₃ is H; or forms part of a pharmacophore having activity at a PPAR or is a cannabinoid pharmacophore substituent;
R₄ is H, methyl, ═O, ═S, ═NH, C₁-C₅ alkyl or C₁-C₅ alkoxy;
R₅ is H, methyl, ═O, ═S, ═NH, C₁-C₅ alkyl or C₁-C₅ alkoxy; and
R₆ is H; or forms part of a pharmacophore having activity at a PPAR or is a cannabinoid pharmacophore substituent;
   with the proviso that

when B is S, R₄ is a lone pair of electrons; and

with the added proviso that

when R₁ forms part of a pharmacophore having activity at a PPAR then R₃ is a cannabinoid pharmacophore substituent and when R₃ forms part of a pharmacophore having activity at a PPAR then R₁ is a cannabinoid pharmacophore substituent,

wherein the PPAR pharmacophore comprises a salicylic acid, an alkoxybenzylacetic acid or an alkoxyphenylacetic acid functionality.

Suitably, an arylcarboxy, C₁-C₈ cycloalkylcarboxy, C₁-C₅ alkylcarboxy, arylcarbamoyl, C₁-C₈ cycloalkylcarbamoyl, C₁-C₅ alkylcarbamoyl groups can also be suitably be used as cannabinoid pharamacophores substituents falling within the meaning of term as described herein. Preferable aryl group derivates include arylalkoxy or arylhalide derivates. Preferably, the cannabinoid pharmacophore substituent may be selected from the group consisting of:

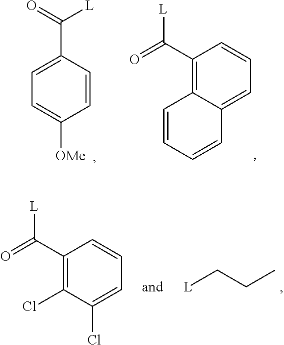

wherein L represents the fused bicyclic linker to which the cannabinoid pharmacophore is bound.

In particular embodiments, the compounds of the invention can be represented by the general formula (I) having activity at least one of a PPAR and a cannabinoid receptor

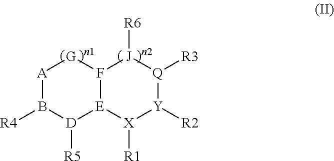

wherein

at least one of the rings is aromatic; at least one of n1 or n2 is 0 or 1; and
provided that at least one ring is aromatic,
   A is CH, N or S; B is C, N or S; D is C or N; E is C or N; F is C or N; G is CH, N or S; X is C or N; Y is C, N or S; Q is C or N; J is CH, N or S; or
provided that at least one ring is not aromatic,
   A is CH, N, NH or S; B is C, N or S; D is C, N or S; E is C or N; F is C or N; G is CH, N, NH or S; X is C or N; Y is C, N or S; Q is C or N; J is CH, N or NH;
      and
R₁ is H or is part of a pharmacophore having activity at a PPAR or is a cannabinoid pharmacophore substituent;
R₂ is H, methyl, ═O, ═S, ═NH or a lone pair of electrons;
R₃ is H; or forms part of a pharmacophore having activity at a PPAR or is a cannabinoid pharmacophore substituent;
R₄ is H, methyl, ═O, ═S, ═NH, C₁-C₅ alkyl or C₁-C₅ alkoxy;
R₅ is H, methyl, ═O, ═S, ═NH, C₁-C₅ alkyl or C₁-C₅ alkoxy; and
R₆ is H; or forms part of a pharmacophore having activity at a PPAR or is a cannabinoid pharmacophore substituent;
   with the proviso that

when B is S, R₄ is a lone pair of electrons; and

with the added proviso that
when R₁ forms part of a pharmacophore having activity at a PPAR then R₃ is a cannabinoid pharmacophore substituent and when R₃ forms part of a pharmacophore having activity at a PPAR then R₁ is a cannabinoid pharmacophore substituent,

wherein the cannabinoid pharmacophore comprises the fused bicyclic ring; and

wherein the PPAR pharmacophore comprises a salicylic acid, an alkoxybenzylacetic acid or an alkoxyphenylacetic acid functionality; and

the PPAR pharmacophore is linked to the bicyclic ring of the cannabinoid pharmacophore through a linker comprising an amine or an amide functional group.

In particular embodiments the PPAR pharmacophore carboxylic acid OH group can be substituted with a C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group. This means that the —OH of —C(O)OH group may be substituted with an alkoxy group such as C₁-C₈alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group.

The alkoxy groups of the alkoxybenzylacetic acid or an alkoxyphenylacetic acid functionality may also comprise an alkoxy group such as C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group. The acid functionality may be —C(O)OH or carboxylic acid esters of same or equivalent bioisosteric groups and derivatives of same.

However, Z comprising a salicylic acid functionality, an alkoxybenzylacetic acid functionality or an alkoxyphenylacetic acid functionality is particularly preferred.

In some embodiments Z further comprises a substitution at the PPAR pharmacophore carboxylic acid OH group, wherein the OH is substituted with a C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group.

Typically, preferred amine or amide linkers can be selected from the group consisting of —X′NR′—, —NR′—, —C(O)NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—,

in which R′ and R″ are independently hydrogen, optionally substituted C₁-C₈ alkyl, C₃-C₁₀ cycloalkyl, aryl, heteroaryl, aralkyl, alkoxy or heteroaralkyl; and

X′ and X″ is independently a bond, —NH—, piperzine, C₁-C₈ allyl, a C₁-C₈ alkylene or C₁-C₈ alkyl.

However, in a particularly preferred embodiment, the amine or amide linker can be selected from the group consisting of —CH₂NH—, —NH—, —C(O)NHNH—, —C(O)NC₂H₄N— and —C(O)NHCH₂CH₂—.

In particularly preferred embodiments, the amine or amide linker can be selected from the group consisting of: —X′NR′—, —NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—, in which R′ is hydrogen, optionally substituted C₁-C₈ alkyl, C₃-C₁₀ cycloalkyl, aryl, heteroaryl, aralkyl, alkoxy or heteroaralkyl; and X′ and X″ is independently a bond, —NH—, piperzine, C₁-C₈ alkyl, a C₁-C₈ alkylene or C₁-C₈ alkyl; R″ is optionally substituted C₁-C₈ alkyl, C₃-C₁₀ cycloalkyl, aryl, heteroaryl, aralkyl, alkoxy or heteroaralkyl;

However, in a particularly preferred embodiment, the amine or amide linker can be selected from the group consisting of —CH₂NH—, —NH—, —C(O)NHNH—, —C(O)NC₂H₄N— and —C(O)NHCH₂CH₂—.

In the most preferred embodiments the amide linker is selected from the group consisting of —C(O)NHNH—, —C(O)NC₂H₄N— and —C(O)NHCH₂CH₂—.

Suitably, an arylcarboxy, C₁-C₈ cycloalkylcarboxy, C₁-C₅ alkylcarboxy, arylcarbamoyl, C₁-C₈ cycloalkylcarbamoyl, C₁-C₅ alkylcarbamoyl groups can also be suitably be used as cannabinoid pharamacophores substituents falling within the meaning of term as described herein. Preferable aryl group derivates include arylalkoxy or arylhalide derivates. Preferably, the cannabinoid pharmacophore substituent may be selected from the group consisting of:

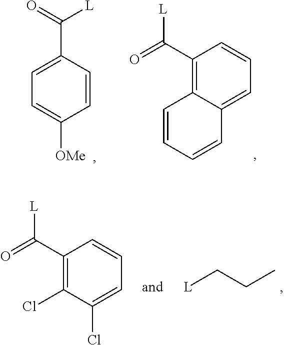

wherein L represents the fused bicyclic linker to which the cannabinoid pharmacophore is bound.

In yet a different aspect relating to the first aspect, there is provided a compound having a general formula V and having activity at, at least one of a PPAR and a cannabinoid receptor, the compound comprising:

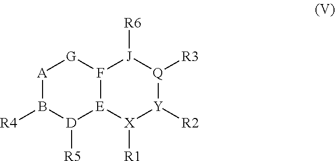

wherein

provided that at least one ring is aromatic,
A is CH, N or S; B is C, N or S; D is C or N; E is C or N; F is C or N; G is CH, N or S; X is C or N; Y is C, N or S; Q is C or N; J is CH, N or S; or

provided that at least one ring is not aromatic,
A is CH, N, NH or S; B is C, N or S; D is C, N or S; E is C or N; F is C or N; G is CH, N, NH or S; X is C or N; Y is C, N or S; Q is C or N; J is CH, N or NH; and

R₁ is H; or forms part of a pharmacophore having activity at a PPAR or a cannabinoid receptor;

R₂ is H, methyl, ═O, ═S, ═NH, C₁-C₅ alkyl, C₁-C₅ alkoxy or a lone pair of electrons;

R₃ is H; or forms part of a pharmacophore having activity at a PPAR or a cannabinoid receptor; and

R₄ is H, methyl, ═O, ═S or ═NH, C₁-C₅ alkyl or C₁-C₅ alkoxy;

R₅ is H, methyl, ═O, ═S or NH, C₁-C₅ alkyl or C₁-C₅ alkoxy;

R₆ is H; or forms part of a pharmacophore having activity at a PPAR or a cannabinoid receptor; provided that
when R₁ forms part of a pharmacophore having activity at a PPAR then R₃ forms part of a pharmacophore having activity at a cannabinoid receptor and when R₃ forms part of a pharmacophore having activity at a PPAR then R₁ forms part of a pharmacophore having activity at a cannabinoid receptor; and
   with the further proviso that
when X is N and R₁ is H then R₂ is ═O and R₃ forms part of a PPAR pharmacophore wherein the PPAR pharmacophore comprises a salicylic acid, an alkoxybenzylacetic acid, or an alkoxyphenylacetic acid functionality.

In particular embodiments the PPAR pharmacophore carboxylic acid OH group can be substituted with a C₁-C₅ alkoxyl, a C₃-C₆ cycloalkoxyl group, a vinyloxyl, a C₃-C₅ allyloxyl, benzoxy, naphthaloxy or benzyloxy group. This means that the —OH of —C(O)OH group may be substituted with an alkoxy group such as C₁-C₅ alkoxyl, a C₃-C₆ cycloalkoxyl group, a vinyloxyl, a C₃-C₅ allyloxyl, benzoxy, naphthaloxy or a benzyloxy group.

The alkoxy groups of the alkoxybenzylacetic acid or a alkoxyphenylacetic acid functionality may also comprise an alkoxy group such as C₁-C₅ alkoxyl, a C₃-C₆ cycloalkoxyl group, a vinyloxyl, a C₃-C₅ allyloxyl, benzoxy, naphthaloxy or a benzyloxy group. The acid functionality may be —C(O)OH or carboxylic acid esters of same.

However, Z comprising a salicylic acid functionality, an alkoxybenzylacetic acid functionality or an alkoxyphenylacetic acid functionality is particularly preferred.

In some embodiments Z further comprises a substitution at the PPAR pharmacophore carboxylic acid OH group, wherein the OH is substituted with a C₁-C₅ alkoxyl, a C₃-C₆ cycloalkoxyl group, a vinyloxyl, a C₃-C₅ allyloxyl, benzoxy, naphthaloxy or benzyloxy group.

Typically, preferred amine or amide linkers can be selected from the group consisting of —X′NR′—, —NR′—, —C(O)NR′—, —C(O)NR′R″—, —NR′C(O)R″—, —C(O)NR′NR″—, —X′NR′R″X″′, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—,

in which R′ and R″ are independently hydrogen, optionally substituted C₁-C₈ alkyl, C₃-C₁₀ cycloalkyl, aryl, heteroaryl, aralkyl, alkoxy or heteroaralkyl; and

X′ and X″ is independently a bond, —NH—, piperzine, C₁-C₈ alkyl, a C₁-C₈ alkylene or C₁-C₈ alkyl.

However, in a particularly preferred embodiment, the amine or amide linker can be selected from the group consisting of —CH₂NH—, —NH—, —C(O)NHNH—, —C(O)NC₂H₄N— and —C(O)NHCH₂CH₂—.

In particularly preferred embodiments, the amine or amide linker can be selected from the group consisting of: —X′NR′—, —NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—, in which R′ is hydrogen, optionally substituted C₁-C₈ alkyl, C₃-C₁₀ cycloalkyl, aryl, heteroaryl, aralkyl, alkoxy or heteroaralkyl; and X′ and X″ is independently a bond, —NH—, piperzine, C₁-C₈ alkyl, a C₁-C₈ alkylene or C₁-C₈ alkyl; R″ is optionally substituted C₁-C₈ alkyl, C₃-C₁₀ cycloalkyl, aryl, heteroaryl, aralkyl, alkoxy or heteroaralkyl;

However, in a particularly preferred embodiment, the amine or amide linker can be selected from the group consisting of —CH₂NH—, —NH—, —C(O)NHNH—, —C(O)NC₂H₄N— and —C(O)NHCH₂CH₂—.

In the most preferred embodiments the amide linker is selected from the group consisting of —C(O)NHNH—, —C(O)NC₂H₄N— and —C(O)NHCH₂CH₂—.

In yet a different aspect, there is provided a compound having a general formula V and having activity at least one of a PPAR and a cannabinoid receptor, the compound comprising:

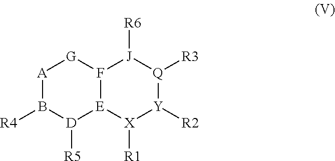

wherein
provided that at least one ring is aromatic,
   A is CH, N or S; B is C, N or S; D is C or N; E is C or N; F is C or N; G is CH, N or S; X is C or N; Y is C, N or S; Q is C or N; J is CH, N or S; or
provided that at least one ring is not aromatic,
   A is CH, N, NH or S; B is C, N or S; D is C, N or S; E is C or N; F is C or N; G is CH, N, NH or S; X is C or N; Y is C, N or S; Q is C or N; J is CH, N or NH; and
R₁ is H; or forms part of a pharmacophore having activity at a PPAR or is a cannabinoid pharmacophore substituent;
R₂ is H, methyl, ═O, ═S, ═NH, C₁-C₅ alkyl, C₁-C₅ alkoxy or a lone pair of electrons;
R₃ is H; or forms part of a pharmacophore having activity at a PPAR or is a cannabinoid pharmacophore substituent; and
R₄ is H, methyl, ═O, ═S or NH, C₁-C₅ alkyl or C₁-C₅ alkoxy;
R₅ is H, methyl, ═O, ═S or NH, C₁-C₅ alkyl or C₁-C₅ alkoxy;
R₆ is H; or forms part of a pharmacophore having activity at a PPAR or is a cannabinoid pharmacophore substituent;
provided that
   when R₁ forms part of a pharmacophore having activity at a PPAR then R₃ is a cannabinoid pharmacophore substituent and when R₃ forms part of a pharmacophore having activity at a PPAR then R₁ is a cannabinoid pharmacophore substituent; and

with the further proviso that

when X is N and R₁ is H then R₂ is ═O;

wherein the cannabinoid pharmacophore comprises the fused bicyclic ring; and

wherein the PPAR pharmacophore comprises a salicylic acid, alkoxybenzylacetic acid or a alkoxyphenylacetic acid functionality; and

the PPAR pharmacophore is linked to the bicyclic ring of the cannabinoid pharmacophore through a linker comprising an amine or an amide functional group.

Suitably, an arylcarboxy, C₁-C₈ cycloalkylcarboxy, C₁-C₅ alkylcarboxy, arylcarbamoyl, C₁-C₈ cycloalkylcarbamoyl, C₁-C₅ alkylcarbamoyl groups can also be suitably be used as cannabinoid pharamacophores substituents falling within the meaning of term as described herein. Preferable aryl group derivates include arylalkoxy or arylhalide derivates. Preferably, the cannabinoid pharmacophore substituent may be selected from the group consisting of:

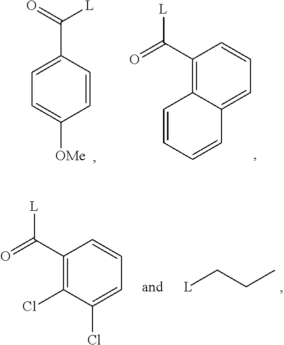

wherein L represents the fused bicyclic linker to which the cannabinoid pharmacophore is bound.

In particular embodiments the PPAR pharmacophore carboxylic acid OH group can be substituted with a C₁-C₅ alkoxyl, a C₃-C₆ cycloalkoxyl group, a vinyloxyl, a C₃-C₅ allyloxyl, benzoxy, naphthaloxy or benzyloxy group. This means that the —OH of —C(O)OH group may be substituted with an alkoxy group such as C₁-C₅ alkoxyl, a C₃-C₆ cycloalkoxyl group, a vinyloxyl, a C₃-C₅ allyloxyl, benzoxy, naphthaloxy or a benzyloxy group.

The alkoxy groups of the alkoxybenzylacetic acid or a alkoxyphenylacetic acid functionality may also comprise an alkoxy group such as C₁-C₅ alkoxyl, a C₃-C₆ cycloalkoxyl group, a vinyloxyl, a C₃-C₅ allyloxyl, benzoxy, naphthaloxy or a benzyloxy group. The acid functionality may be —C(O)OH or carboxylic acid esters of same.

However, Z comprising a salicylic acid functionality, an alkoxybenzylacetic acid functionality or an alkoxyphenylacetic acid functionality is particularly preferred.

In some embodiments Z further comprises a substitution at the PPAR pharmacophore carboxylic acid OH group, wherein the OH is substituted with a C₁-C₅ alkoxyl, a C₃-C₆ cycloalkoxyl group, a vinyloxyl, a C₃-C₅ allyloxyl, benzoxy, naphthaloxy or benzyloxy group.

Typically, preferred amine or amide linkers can be selected from the group consisting of —X′NR′—, —NR′—, —C(O)NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—,

in which R′ and R″ are independently hydrogen, optionally substituted C₁-C₈ alkyl, C₃-C₁₀ cycloalkyl, aryl, heteroaryl, aralkyl, alkoxy or heteroaralkyl; and

X′ and X″ is independently a bond, —NH—, piperzine, C₁-C₈ allyl, a C₁-C₈ alkylene or C₁-C₈ alkyl.

In particularly preferred embodiments, the amine or amide linker can be selected from the group consisting of: —X′NR′—, —NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—, in which R′ is hydrogen, optionally substituted C₁-C₈ alkyl, C₃-C₁₀ cycloalkyl, aryl, heteroaryl, aralkyl, alkoxy or heteroaralkyl; and X′ and X″ is independently a bond, —NH—, piperzine, C₁-C₈ alkyl, a C₁-C₈ alkylene or C₁-C₈ alkyl; R″ is optionally substituted C₁-C₈ alkyl, C₃-C₁₀ cycloalkyl, aryl, heteroaryl, aralkyl, alkoxy or heteroaralkyl;

However, in a particularly preferred embodiment, the amine or amide linker can be selected from the group consisting of —CH₂NH—, —NH—, —C(O)NHNH—, —C(O)NC₂H₄N— and —C(O)NHCH₂CH₂—.

In the most preferred embodiments the amide linker is selected from the group consisting of —C(O)NHNH—, —C(O)NC₂H₄N— and —C(O)NHCH₂CH₂—.

Preferred compounds of the second aspect of the invention have the general formula (II)

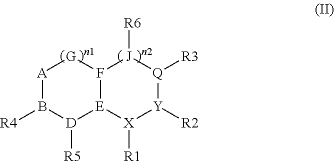

wherein at least one of the fused bicycle rings is aromatic;
n¹ is 0 or 1;
n² is 0 or 1; wherein at least one of n1 or n2 is 1; and at least one of the fused bicycle ring is aromatic; and wherein:
A is CH, N or S; B is C, N or S; D is C or N; E is C or N; F is C or N; G is CH, N or S; X is C or N; Y is C, N or S; Q is C or N; J is CH, N or S; or
A is CH, N, NH or S; B is C, N or S; D is C, N or S; E is C or N; F is C or N; G is CH, N, NH or S; X is C or N; Y is C, N or S; Q is C or N; J is CH, N or NH;
   and
one of R₁, R₃ or R₆ is R₁₄, wherein R₁₄ is the amide or amine linkage covalently bound to the PPAR pharmacophore;

wherein the PPAR pharmacophore comprises a salicylic acid, an alkoxybenzylacetic acid or an alkoxyphenylacetic acid functionality; and
R₁₅ is a cannabinoid pharmacophore substituent selected from the group consisting of:

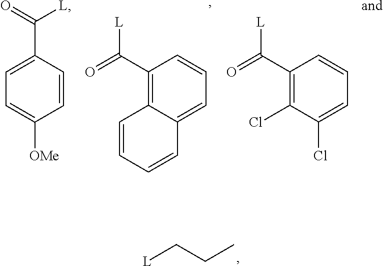

wherein L indicates the point of attachment;
R₁ is selected from H, C₁-C₈alkyl, R₁₅ or R₁₄;
R₂ is H, methyl, ═O, ═S, ═NH or a lone pair of electrons;
R₃ is H, R₁₄, or R₁₅; and
R₆ is H, R₁₄, or R₁₅;
R₄ is H, methyl, ═O, ═S, ═NH, C₁-C₈ alkyl or C₁-C₈ alkoxy;
R₅ is H, methyl, ═O, ═S, ═NH, C₁-C₈ alkyl or C₁-C₈ alkoxy;
   with the proviso that,
when B is S, R₄ is a lone pair of electrons; and

when R₁ is R₁₄ then R₃ is R₁₅ and when R₃ is R₁₄ then R₁ is R₁₅.

In particular embodiments the PPAR pharmacophore carboxylic acid OH group can be substituted with a C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group. This means that the —OH of —C(O)OH group may be substituted with an alkoxy group such as C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group.

The alkoxy groups of the alkoxybenzylacetic acid or a alkoxyphenylacetic acid functionality may also comprise an alkoxy group such as C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group. The acid functionality may be —C(O)OH or carboxylic acid esters of same.

However, Z comprising a salicylic acid functionality, an alkoxybenzylacetic acid functionality or an alkoxyphenylacetic acid functionality is particularly preferred.

In some embodiments Z further comprises a substitution at the PPAR pharmacophore carboxylic acid OH group, wherein the OH is substituted with a C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group.

Typically, preferred amine or amide linkers can be selected from the group consisting of —X′NR′—, —NR′—, —C(O)NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—,

in which R′ and R″ are independently hydrogen, optionally substituted C₁-C₈ alkyl, C₃-C₁₀ cycloalkyl, aryl, heteroaryl, aralkyl, alkoxy or heteroaralkyl; and

X′ and X″ is independently a bond, —NH—, piperzine, C₁-C₈ allyl, a C₁-C₈ alkylene or C₁-C₈ alkyl.

In particularly preferred embodiments, the amine or amide linker can be selected from the group consisting of: —X′NR′—, —NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—, in which R′ is hydrogen, optionally substituted C₁-C₈ alkyl, C₃-C₁₀ cycloalkyl, aryl, heteroaryl, aralkyl, alkoxy or heteroaralkyl; and X′ and X″ is independently a bond, —NH—, piperzine, C₁-C₈ allyl, a C₁-C₈ alkylene or C₁-C₈ alkyl; R″ is optionally substituted C₁-C₈ alkyl, C₃-C₁₀ cycloalkyl, aryl, heteroaryl, aralkyl, alkoxy or heteroaralkyl;

However, in a particularly preferred embodiment, the amine or amide linker can be selected from the group consisting of —CH₂NH—, —NH—, —C(O)NHNH—, —C(O)NC₂H₄N— and —C(O)NHCH₂CH₂—.

In the most preferred embodiments the amide linker is selected from the group consisting of —C(O)NHNH—, —C(O)NC₂H₄N— and —C(O)NHCH₂CH₂—.

Other preferred compounds relating to the second aspect of the invention have the general formula (II)

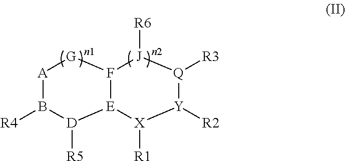

wherein at least one of the fused bicycle rings is aromatic;
n¹ is 0 or 1;
n² is 0 or 1; wherein at least one of n1 or n2 is 1; and at least one of the fused bicycle ring is aromatic; and wherein:
A is CH, N or S; B is C, N or S; D is C or N; E is C or N; F is C or N; G is CH, N or S; X is C or N; Y is C, N or S; Q is C or N; J is CH, N or S; or
A is CH, N, NH or S; B is C, N or S; D is C, N or S; E is C or N; F is C or N; G is CH, N, NH or S; X is C or N; Y is C, N or S; Q is C or N; J is CH, N or NH;
   and

one of R₁, R₃ or R₆ is R₁₄, wherein R₁₄ is the amide or amine linkage covalently bound to the PPAR pharmacophore, wherein the PPAR pharmacophore comprises a salicylic acid, an alkoxybenzylacetic acid or an alkoxyphenylacetic acid functionality; and

wherein the amine or amide linkers can be selected from the group consisting of —X′NR′—, —NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—, in which R′ is hydrogen, optionally substituted C₁-C₈ alkyl, C₃-C₁₀ cycloalkyl, aryl, heteroaryl, aralkyl, alkoxy or heteroaralkyl; and X′ and X″ is independently a bond, —NH—, piperzine, C₁-C₈ alkyl, a C₁-C₈ alkylene or C₁-C₈ alkyl; R″ is optionally substituted C₁-C₈ alkyl, C₃-C₁₀ cycloalkyl, aryl, heteroaryl, aralkyl, alkoxy or heteroaralkyl;

R₁₅ is selected from the group consisting of:

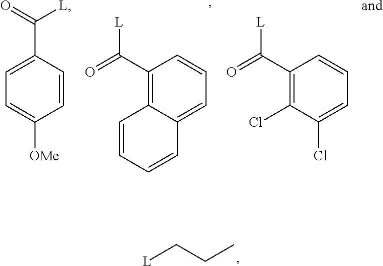

wherein L indicates the point of attachment;
R₁ is selected from H, C₁-C₈alkyl, R₁₅ or R₁₄;
R₂ is H, methyl, ═O, ═S, ═NH or a lone pair of electrons;
R₃ is H, R₁₄, or R₁₅; and
R₆ is H, R₁₄, or R₁₅;
R₄ is H, methyl, ═O, ═S, ═NH, C₁-C₈ alkyl or C₁-C₈ alkoxy;
R₅ is H, methyl, ═O, ═S, ═NH, C₁-C₈alkyl or C₁-C₈ alkoxy;
   with the proviso that,
when B is S, R₄ is a lone pair of electrons; and
when R₁ is R₁₄ then R₃ is R₁₅ and when R₃ is R₁₄ then R₁ is R.

In particular embodiments the PPAR pharmacophore carboxylic acid OH group can be substituted with a C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group. This means that the —OH of —C(O)OH group may be substituted with an alkoxy group such as C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group.

The alkoxy groups of the alkoxybenzylacetic acid or a alkoxyphenylacetic acid functionality may also comprise an alkoxy group such as C₁-C₈alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group. The acid functionality may be —C(O)OH or carboxylic acid esters of same.

However, Z comprising a salicylic acid functionality, an alkoxybenzylacetic acid functionality or an alkoxyphenylacetic acid functionality is particularly preferred.

In some embodiments Z further comprises a substitution at the PPAR pharmacophore carboxylic acid OH group, wherein the OH is substituted with a C₁-C₅ alkoxyl, a C₃-C₆ cycloalkoxyl group, a vinyloxyl, a C₃-C₅ allyloxyl, benzoxy, naphthaloxy or benzyloxy group.

Suitably, an arylcarboxy, C₁-C₈ cycloalkylcarboxy, C₁-C₅ alkylcarboxy, arylcarbamoyl, C₁-C₈ cycloalkylcarbamoyl, C₁-C₅ alkylcarbamoyl groups can also be suitably be used as cannabinoid pharamacophores substituents falling within the meaning of term as described herein. Preferable aryl group derivates include arylalkoxy or arylhalide derivates. Preferably, the cannabinoid pharmacophore substituent may be selected from the group consisting of:

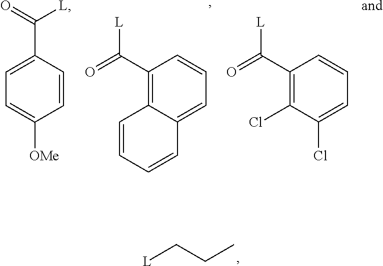

wherein L represents the fused bicyclic linker to which the cannabinoid pharmacophore is bound.

In another aspect relating to the first aspect, there is provided a compound having a general formula IIIA or IIIB and having activity at, at least one of a PPAR and a cannabinoid receptor, the compound comprising:

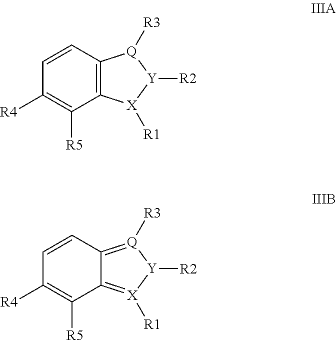

wherein according to IIIA the benzene ring is aromatic or according to IIIB the heterocylic ring is aromatic; and

X is C, N or S; Y is C, N or S; Q is C, N or S;

R₁ is H; or forms part of a pharmacophore having activity at a PPAR or a cannabinoid receptor;

R₂ is H, methyl, ═O, ═S, ═NH, C₁-C₅ alkyl, C₁-C₅ alkoxy or a lone pair of electrons;

R₃ is H; or forms part of a pharmacophore having activity at a PPAR or a cannabinoid receptor;

R₄ is H, methyl, ═O, ═S, ═NH, C₁-C₅ alkyl or C₁-C₅ alkoxy;

R₅ is H, methyl, ═O, ═S, ═NH, C₁-C₅ alkyl or C₁-C₅ alkoxy;

with the proviso that
when Y is C, R₂ is H, ═O, ═S, ═NH; or when Y is N, R₂ is H or a lone pair of electrons; or when Y is S, R₂ is a lone pair of electrons; and
   with the further proviso that
when R₁ forms part of a pharmacophore having activity at a PPAR then R₃ forms part of a pharmacophore having activity at a cannabinoid receptor and when R₃ forms part of a pharmacophore having activity at a PPAR then R₁ forms part of a pharmacophore having activity at a cannabinoid receptor
   wherein the PPAR pharmacophore comprises a salicylic acid, an alkoxybenzylacetic acid, or an alkoxyphenylacetic acid functionality.

In another aspect there is provided a compound having a general formula IIIA or IIIB and having activity at, at least one of a PPAR and a cannabinoid receptor, the compound comprising:

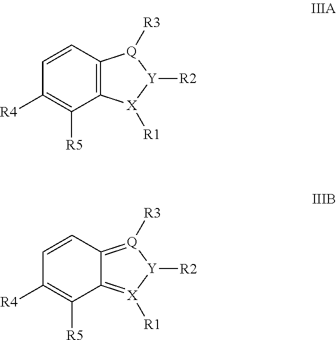

wherein according to IIIA the benzene ring is aromatic or according to IIIB the heterocylic ring is aromatic; and
X is C, N or S; Y is C, N or S; Q is C, N or S;
R₁ is H; or forms part of a pharmacophore having activity at a PPAR or is a cannabinoid pharmacophore substituent;
R₂ is H, methyl, ═O, ═S, ═NH, C₁-C₅ alkyl, C₁-C₅ alkoxy or a lone pair of electrons;
R₃ is H; or forms part of a pharmacophore having activity at a PPAR or is a cannabinoid pharmacophore substituent;
R₄ is H, methyl, ═O, ═S, ═NH, C₁-C₅ alkyl or C₁-C₅ alkoxy;
R₅ is H, methyl, ═O, ═S, ═NH, C₁-C₅ alkyl or C₁-C₅ alkoxy;
   with the proviso that
when Y is C, R₂ is H, ═O, ═S, ═NH; or when Y is N, R₂ is H or a lone pair of electrons; or when Y is S, R₂ is a lone pair of electrons; and
   with the further proviso that
when R₁ forms part of a pharmacophore having activity at a PPAR then R₃ is a cannabinoid pharmacophore substituent and when R₃ forms part of a pharmacophore having activity at a PPAR then R₁ is a cannabinoid pharmacophore substituent wherein the cannabinoid pharmacophore comprises the fused bicyclic ring; and
wherein the PPAR pharmacophore comprises a salicylic acid, an alkoxybenzylacetic acid or an alkoxyphenylacetic acid functionality; and
   the PPAR pharmacophore is linked to the bicyclic ring of the cannabinoid pharmacophore through a linker comprising an amine or an amide functional group.

In particular embodiments the PPAR pharmacophore carboxylic acid OH group can be substituted with a C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group. This means that the —OH of —C(O)OH group may be substituted with an alkoxy group such as C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group.

The alkoxy groups of the alkoxybenzylacetic acid or a alkoxyphenylacetic acid functionality may also comprise an alkoxy group such as C₁-C₈alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group. The acid functionality may be —C(O)OH or carboxylic acid esters of same.

However, Z comprising a salicylic acid functionality, an alkoxybenzylacetic acid functionality or an alkoxyphenylacetic acid functionality is particularly preferred.

In some embodiments Z further comprises a substitution at the PPAR pharmacophore carboxylic acid OH group, wherein the OH is substituted with a C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group.

Suitably, an arylcarboxy, C₁-C₈ cycloalkylcarboxy, C₁-C₅ alkylcarboxy, arylcarbamoyl, C₁-C₈ cycloalkylcarbamoyl, C₁-C₅ alkylcarbamoyl groups can also be suitably be used as cannabinoid pharamacophores substituents falling within the meaning of term as described herein. Preferable aryl group derivates include arylalkoxy or arylhalide derivates. Preferably, the cannabinoid pharmacophore substituent may be selected from the group consisting of:

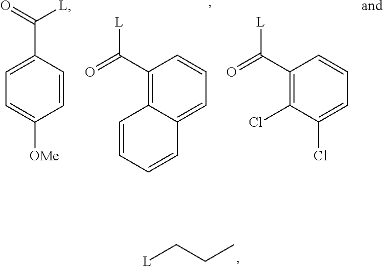

wherein L represents the fused bicyclic linker to which the cannabinoid pharmacophore is bound.

Typically, preferred amine or amide linkers can be selected from the group consisting of —X′NR′—, —NR′—, —C(O)NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—,

in which R′ and R″ are independently hydrogen, optionally substituted C₁-C₈ alkyl, C₃-C₁₀ cycloalkyl, aryl, heteroaryl, aralkyl, alkoxy or heteroaralkyl; and

X′ and X″ is independently a bond, —NH—, piperzine, C₁-C₈ allyl, a C₁-C₈ alkylene or C₁-C₈ alkyl.

In particularly preferred embodiments, the amine or amide linker can be selected from the group consisting of: —X′NR′—, —NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—, in which R′ is hydrogen, optionally substituted C₁-C₈ alkyl, C₃-C₁₀ cycloalkyl, aryl, heteroaryl, aralkyl, alkoxy or heteroaralkyl; and X′ and X″ is independently a bond, —NH—, piperzine, C₁-C₈ allyl, a C₁-C₈ alkylene or C₁-C₈ alkyl; R″ is optionally substituted C₁-C₈ alkyl, C₃-C₁₀ cycloalkyl, aryl, heteroaryl, aralkyl, alkoxy or heteroaralkyl;

However, in a particularly preferred embodiment, the amine or amide linker can be selected from the group consisting of —CH₂NH—, —NH—, —C(O)NHNH—, —C(O)NC₂H₄N— and —C(O)NHCH₂CH₂—.

In the most preferred embodiments the amide linker is selected from the group consisting of —C(O)NHNH—, —C(O)NC₂H₄N— and —C(O)NHCH₂CH₂—.

In a different aspect there is provided a compound having a general formula IVA or IVB and having activity at, at least one of a PPAR and a cannabinoid receptor, the compound comprising:

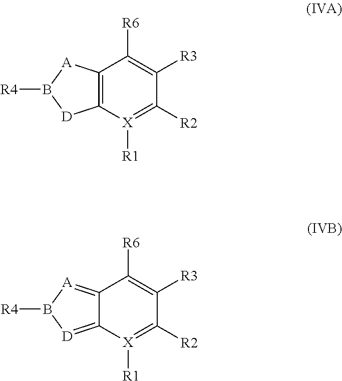

wherein

when the six membered ring is aromatic;
A is CH, CH₂, N, NH or S; B is C, CH, N or S; D is CH, CH₂, N, NH or S; X is C or N;

when the five membered ring is aromatic;
A is CH, N or S; B is C, N or S; D is CH, N or S; X is C, CH or N;
   and
R₁ is H; or forms part of a pharmacophore having activity at a PPAR or a cannabinoid receptor;
R₂ is H, methyl, ═O, ═S, ═NH, C₁-C₅ alkyl, C₁-C₅ alkoxy or a lone pair of electrons;
R₃ is H; or forms part of a pharmacophore having activity at a PPAR or a cannabinoid receptor;
R₄ is H, methyl, ═O, ═S, ═NH; and
R₆ is H; or forms part of a pharmacophore having activity at a PPAR or a cannabinoid receptor;
   with the proviso that
when B is C, R₂ is H, ═O, ═S, ═NH; or when B is N, R₂ is H or a lone pair of electrons; or when B is S, R₂ is a lone pair of electrons; and
   with the further proviso that
when R₁ forms part of a pharmacophore having activity at a PPAR then R₃ forms part of a pharmacophore having activity at a cannabinoid receptor and when R₃ forms part of a pharmacophore having activity at a PPAR then R₁ forms part of a pharmacophore having activity at a cannabinoid receptor;
   with the further proviso that
when X is N and R₁ is H then R₂ is ═O and R₃ forms part of a pharmacophore comprising a salicylic acid functionality, an alkoxybenzylacetic acid, or an alkoxyphenylacetic acid functionality.

In particular embodiments the PPAR pharmacophore carboxylic acid OH group can be substituted with a C₁-C₅ alkoxyl, a C₃-C₆ cycloalkoxyl group, a vinyloxyl, a C₃-C₅ allyloxyl, benzoxy, naphthaloxy or benzyloxy group. This means that the —OH of —C(O)OH group may be substituted with an alkoxy group such as C₁-C₅ alkoxyl, a C₃-C₆ cycloalkoxyl group, a vinyloxyl, a C₃-C₅ allyloxyl, benzoxy, naphthaloxy or a benzyloxy group.

The alkoxy groups of the alkoxybenzylacetic acid or a alkoxyphenylacetic acid functionality may also comprise an alkoxy group such as C₁-C₅ alkoxyl, a C₃-C₆ cycloalkoxyl group, a vinyloxyl, a C₃-C₅ allyloxyl, benzoxy, naphthaloxy or a benzyloxy group. The acid functionality may be —C(O)OH or carboxylic acid esters of same.

However, Z comprising a salicylic acid functionality, an alkoxybenzylacetic acid functionality or an alkoxyphenylacetic acid functionality is particularly preferred.

In some embodiments Z further comprises a substitution at the PPAR pharmacophore carboxylic acid OH group, wherein the OH is substituted with a C₁-C₅ alkoxyl, a C₃-C₆ cycloalkoxyl group, a vinyloxyl, a C₃-C₅ allyloxyl, benzoxy, naphthaloxy or benzyloxy group.

Typically, preferred amine or amide linkers can be selected from the group consisting of —X′NR′—, —NR′—, —C(O)NR′—, —C(O)NR′R″—, —NR′C(O)R″−, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—,

in which R′ and R″ are independently hydrogen, optionally substituted C₁-C₈ alkyl, C₃-C₁₀ cycloalkyl, aryl, heteroaryl, aralkyl, alkoxy or heteroaralkyl; and

X′ and X″ is independently a bond, —NH—, piperzine, C₁-C₈ allyl, a C₁-C₈ alkylene or C₁-C₈ alkyl.

However, in a particularly preferred embodiment, the amine or amide linker can be selected from the group consisting of —CH₂NH—, —NH—, —C(O)NHNH—, —C(O)NC₂H₄N— and —C(O)NHCH₂CH₂—.

In particularly preferred embodiments, the amine or amide linker can be selected from the group consisting of: —X′NR′—, —NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—, in which R′ is hydrogen, optionally substituted C₁-C₈ alkyl, C₃-C₁₀ cycloalkyl, aryl, heteroaryl, aralkyl, alkoxy or heteroaralkyl; and X′ and X″ is independently a bond, —NH—, piperzine, C₁-C₈ allyl, a C₁-C₈ alkylene or C₁-C₈ alkyl; R″ is optionally substituted C₁-C₈ alkyl, C₃-C₁₀ cycloalkyl, aryl, heteroaryl, aralkyl, alkoxy or heteroaralkyl;

However, in a particularly preferred embodiment, the amine or amide linker can be selected from the group consisting of —CH₂NH—, —NH—, —C(O)NHNH—, —C(O)NC₂H₄N— and —C(O)NHCH₂CH₂—.

In the most preferred embodiments the amide linker is selected from the group consisting of —C(O)NHNH—, —C(O)NC₂H₄N— and —C(O)NHCH₂CH₂—.

In a different aspect there is provided a compound having a general formula IVA or IVB and having activity at least one of a PPAR and a cannabinoid receptor, the compound comprising:

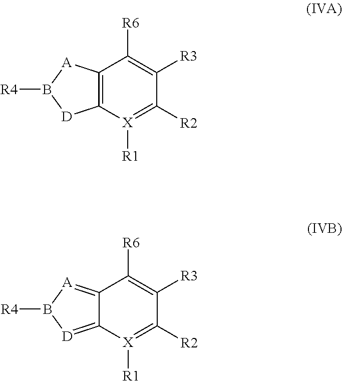

wherein

when the six membered ring is aromatic;
A is CH, CH₂, N, NH or S; B is C, CH, N or S; D is CH, CH₂, N, NH or S; X is C or N;

when the five membered ring is aromatic;
A is CH, N or S; B is C, N or S; D is CH, N or S; X is C, CH or N;
   and

R₁ is H; or forms part of a pharmacophore having activity at a PPAR or is a cannabinoid pharmacophore substituent;

R₂ is H, methyl, ═O, ═S, ═NH, C₁-C₅ alkyl, C₁-C₅ alkoxy or a lone pair of electrons;

R₃ is H; or forms part of a pharmacophore having activity at a PPAR or is a cannabinoid pharmacophore substituent;

R₄ is H, methyl, ═O, ═S, ═NH; and

R₆ is H; or forms part of a pharmacophore having activity at a PPAR or is a cannabinoid pharmacophore substituent;

with the proviso that
when B is C, R₂ is H, ═O, ═S, ═NH; or when B is N, R₂ is H or a lone pair of electrons; or when B is S, R₂ is a lone pair of electrons; and
   with the further proviso that
when R₁ forms part of a pharmacophore having activity at a PPAR then R₃ is a cannabinoid pharmacophore substituent and when R₃ forms part of a pharmacophore having activity at a PPAR then R₁ is a cannabinoid pharmacophore substituent;
   with the further proviso that
   when X is N and R₁ is H then R₂ is ═O,

wherein the cannabinoid pharmacophore comprises the fused bicyclic ring; and

wherein the PPAR pharmacophore comprises a salicylic acid, alkoxybenzylacetic acid or a alkoxyphenylacetic acid functionality; and

the PPAR pharmacophore is linked to the bicyclic ring of the cannabinoid pharmacophore through a linker comprising an amine or an amide functional group.

In particular embodiments the PPAR pharmacophore carboxylic acid OH group can be substituted with a C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group. This means that the —OH of —C(O)OH group may be substituted with an alkoxy group such as C₁-C₈alkoxy, C₃-C₆ cycloalkoxyl (—OR^{alk}(cyc) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group.

The alkoxy groups of the alkoxybenzylacetic acid or a alkoxyphenylacetic acid functionality may also comprise an alkoxy group such as C₁-C₈alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group. The acid functionality may be —C(O)OH or carboxylic acid esters of same.

However, Z comprising a salicylic acid, an alkoxybenzylacetic acid or an alkoxyphenylacetic acid functionality is particularly preferred.

In some embodiments Z further comprises a substitution at the PPAR pharmacophore carboxylic acid OH group, wherein the OH is substituted with a C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group.

Suitably, an arylcarboxy, C₁-C₈ cycloalkylcarboxy, C₁-C₅ alkylcarboxy, arylcarbamoyl, C₁-C₈ cycloalkylcarbamoyl, C₁-C₅ alkylcarbamoyl groups can also be suitably be used as cannabinoid pharamacophores substituents falling within the meaning of term as described herein. Preferable aryl group derivates include arylalkoxy or arylhalide derivates. Preferably, the cannabinoid pharmacophore substituent may be selected from the group consisting of:

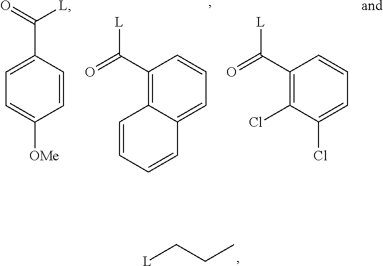

wherein L represents the fused bicyclic linker to which the cannabinoid pharmacophore is bound.

Typically, preferred amine or amide linkers can be selected from the group consisting of —X′NR′—, —NR′—, —C(O)NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—,

in which R′ and R″ are independently hydrogen, optionally substituted C₁-C₈ alkyl, C₃-C₁₀ cycloalkyl, aryl, heteroaryl, aralkyl, alkoxy or heteroaralkyl; and

X′ and X″ is independently a bond, —NH—, piperzine, C₁-C₈ allyl, a C₁-C₈ alkylene or C₁-C₈ alkyl.

In particularly preferred embodiments, the amine or amide linker can be selected from the group consisting of: —X′NR′—, —NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—, in which R′ is hydrogen, optionally substituted C₁-C₈ alkyl, C₃-C₁₀ cycloalkyl, aryl, heteroaryl, aralkyl, alkoxy or heteroaralkyl; and X′ and X″ is independently a bond, —NH—, piperzine, C₁-C₈ allyl, a C₁-C₈ alkylene or C₁-C₈ alkyl; R″ is optionally substituted C₁-C₈ alkyl, C₃-C₁₀ cycloalkyl, aryl, heteroaryl, aralkyl, alkoxy or heteroaralkyl;

However, in a particularly preferred embodiment, the amine or amide linker can be selected from the group consisting of —CH₂NH—, —NH—, —C(O)NHNH—, —C(O)NC₂H₄N— and —C(O)NHCH₂CH₂—.

In the most preferred embodiments the amide linker is selected from the group consisting of —C(O)NHNH—, —C(O)NC₂H₄N— and —C(O)NHCH₂CH₂—.

In a preferred embodiment relating to the second aspect, compounds of the invention have general formula (V*):

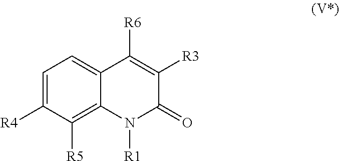

wherein
R₁ is H, or C₁-C₈alkyl or a cannabinoid pharmacophore substituent;

R₃ is a cannabinoid pharmacophore substituent or is —R₁₆-R₁₄; wherein R₁₆ is an amide or amide linker selected from the group consisting of: —X′NR′—, —NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″-, —X′C(O)NR′X″—, —X″R″NC(O)NR′X′—and —X″OC(O)NR′X′—, in which R′ is hydrogen, optionally substituted C₁-C₈ alkyl, C₃-C₁₀ cycloalkyl, aryl, heteroaryl, aralkyl, alkoxy or heteroaralkyl; and X′ and X″ is independently a bond, —NH—, piperzine, C₁-C₈ allyl, a C₁-C₈ alkylene or C₁-C₈ alkyl; R″ is optionally substituted C₁-C₈ alkyl, C₃-C₁₀ cycloalkyl, aryl, heteroaryl, aralkyl, alkoxy or heteroaralkyl; and

R₁₄ is selected from the group consisting of:

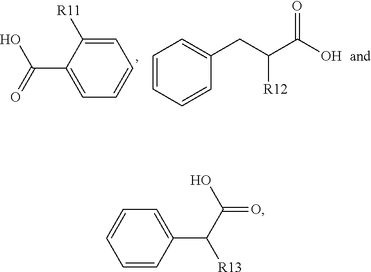

wherein:
R₁₁, R₁₂, and R₁₃ are each independently selected from the group consisting of: OH, C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group;
R₄ is C₁-C₈alkoxy, C₁-C₈alkyl or H;
R₅ is H, methyl, ═O, ═S or NH, C₁-C₅ alkyl or C₁-C₅ alkoxy;
R₆ is H or a cannabinoid pharmacophore substituent or —R₁₆-R₁₄.

Suitably, an arylcarboxy, C₁-C₈ cycloalkylcarboxy, C₁-C₅ alkylcarboxy, arylcarbamoyl, C₁-C₈ cycloalkylcarbamoyl, C₁-C₅ alkylcarbamoyl groups can also be suitably be used as cannabinoid pharamacophores substituents falling within the meaning of term as described herein. Preferable aryl group derivates include arylalkoxy or arylhalide derivates. Preferably, the cannabinoid pharmacophore substituent may be selected from the group consisting of:

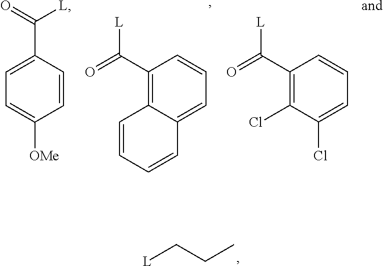

wherein L represents the fused bicyclic linker to which the cannabinoid pharmacophore is bound.

The alkoxy groups of the alkoxybenzylacetic acid or a alkoxyphenylacetic acid functionality may also comprise an alkoxy group such as C₁-C₈alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group. The acid functionality may be —C(O)OH or carboxylic acid esters of same.

However, Z comprising a salicylic acid functionality, an alkoxybenzylacetic acid functionality or an alkoxyphenylacetic acid functionality is particularly preferred.

In some embodiments Z further comprises a substitution at the PPAR pharmacophore carboxylic acid OH group, wherein the OH is substituted with a C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group.

The compound according to any of the preceding claims with general formula (V*):

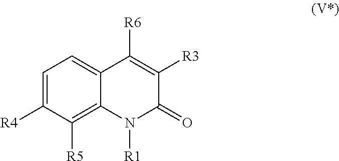

wherein
R₁ is H, or C₁-C₈alkyl, or a cannabinoid pharmacophore substituent;
R₃ is a cannabinoid pharmacophore substituent or is —R₁₆-R₁₄; wherein R₁₆ is an amide or amide linker selected from the group consisting of -alkylene-NR′—, —NR′—, —C(O)—NR′-alkylene-, NR′—C(O)-alkylene-, —C(O)—NR′NR′—, wherein R′ is H or C₁-C₈ alkyl,
R₁₄ is selected from the group consisting of:

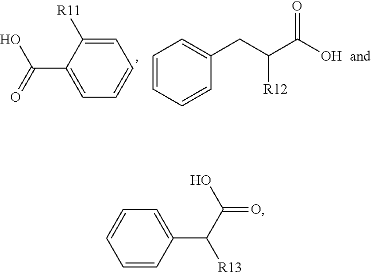

wherein:
R₁₁, R₁₂, and R₁₃ are each independently selected from the group consisting of: OH, C₁-C₈alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group;
R₄ is C₁-C₈alkoxy, C₁-C₈alkyl or H;
R₅ is H, methyl, ═O, ═S or NH, C₁-C₅ alkyl or C₁-C₅ alkoxy;
R₆ is H or a cannabinoid pharmacophore substituent.

Suitably, an arylcarboxy, C₁-C₈ cycloalkylcarboxy, C₁-C₅ alkylcarboxy, arylcarbamoyl, C₁-C₈ cycloalkylcarbamoyl, C₁-C₅ alkylcarbamoyl groups can also be suitably be used as cannabinoid pharamacophores substituents falling within the meaning of term as described herein. Preferable aryl group derivates include arylalkoxy or arylhalide derivates. Preferably, the cannabinoid pharmacophore substituent may be selected from the group consisting of:

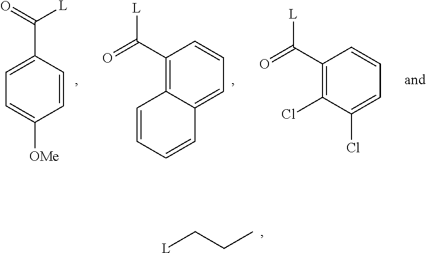

wherein L represents the fused bicyclic linker to which the cannabinoid pharmacophore is bound.

In another particular embodiment, there is provided a compound having general formula (VI)

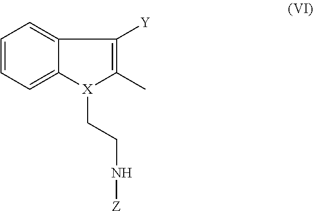

wherein
X is C, N or S; and
Y is a naphthoyl, arylcarboxy, cycloalkylcarboxy, arylcarbamoyl, cycloalkylcarbamoyl or alkylcarbamoyl group; and
Z has salicylic acid functionality, an alkoxybenzylacetic acid functionality or an alkoxyphenylacetic acid functionality.

In some embodiments Z further comprises a substitution at the PPAR pharmacophore carboxylic acid OH group, wherein the OH is substituted with a C₁-C₅ alkoxyl, a C₃-C₆ cycloalkoxyl group, a vinyloxyl, a C₃-C₅ allyloxyl, benzoxy, naphthaloxy or benzyloxy group.

However, Z comprising a salicylic acid, alkoxybenzylacetic acid or a alkoxyphenylacetic acid functionality are particularly preferred.

In a related embodiment there is provided a compound having general formula (VII)

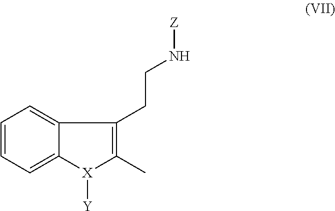

wherein
X is C, N or S;
Y is a naphthoyl, arylcarboxy, cycloalkylcarboxy, arylcarbamoyl, cycloalkylcarbamoyl or alkylcarbamoyl group; and
Z has salicylic acid, alkoxybenzylacetic acid or a alkoxyphenylacetic acid functionality.

In a related embodiment this is provided a compound having general formula (VII*)

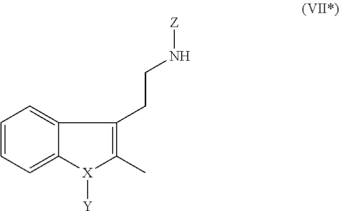

wherein
X is C, N or S;
Y is a cannabinoid pharmacophore substituent selected from the group consisting of a naphthoyl, arylcarboxy, cycloalkylcarboxy, arylcarbamoyl, cycloalkylcarbamoyl or an alkylcarbamoyl group; and
Z is a salicylic acid functionality, an alkoxybenzylacetic acid functionality or an alkoxyphenylacetic acid functionality.

In another embodiment still, there is provided a compound having general formula (VIII)

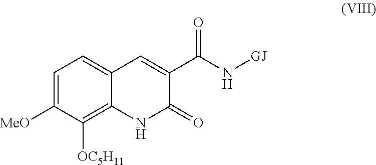

wherein
G is a C₁-C₃ alkyl group; and

J is salicylic acid or an alkoxybenzylacetic acid or an alkoxyphenylacetic acid functionality. The acid functionality may be —C(O)OH or carboxylic acid esters of same.

In some embodiments J further comprises a substitution at the PPAR pharmacophore carboxylic acid OH group, wherein the OH is substituted with an alkoxy group such as a C₁-C₅ alkoxyl, a C₃-C₈ cycloalkoxyl group, a vinyloxyl, a C₃-C₅ allyloxyl, benzoxy, naphthaloxy or a benzyloxy group.

However, compounds wherein J comprises a salicylic acid group, an alkoxybenzylacetic acid or an alkoxyphenylacetic acid functionality are particularly preferred. The acid functionality may be —C(O)OH or carboxylic acid esters of same.

In another embodiment still, there is provided a compound having general formula (VIII)

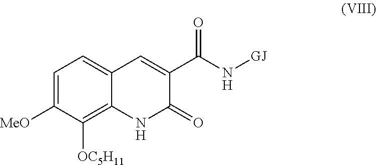

wherein
G is a C₁-C₈ alkyl group; and

J is salicylic acid functionality or an alkoxybenzylacetic acid functionality or an alkoxyphenylacetic acid functionality. The acid functionality may be —C(O)OH or carboxylic acid esters of same.

In some embodiments J further comprises a substitution at the PPAR pharmacophore carboxylic acid OH group, wherein the OH is substituted with an alkoxy group such as a C₁-C₈ alkoxy, C₃-C₆ cycloalkoxyl (—OR^{alk}(cyc) group, a vinyloxyl (—OCH₂CH₂), a C₃-C₅ allyloxyl, benzoxy (—OPh), naphthaloxy (—ONp), benzyloxy (—OCH₂Ph) or a phenylphenoxy (—OPhPh) group.

Particularly preferred compounds of the invention, having agonist activity at, at least one of a PPAR and a cannabinoid receptor may be selected from the group consisting of:

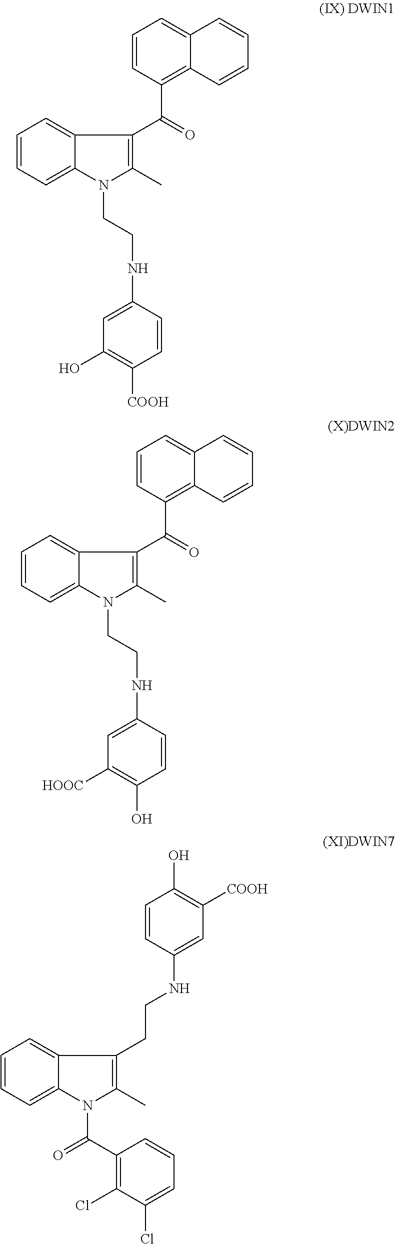

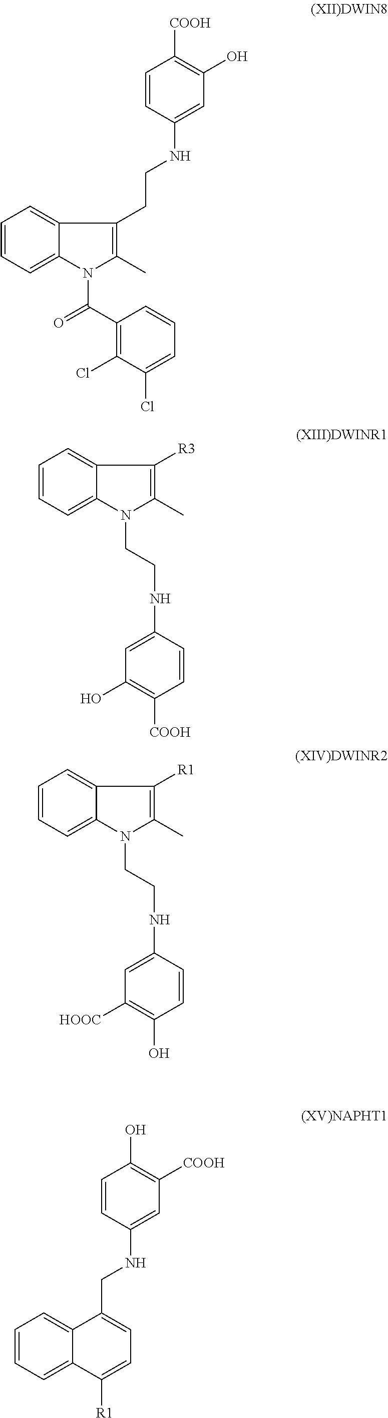

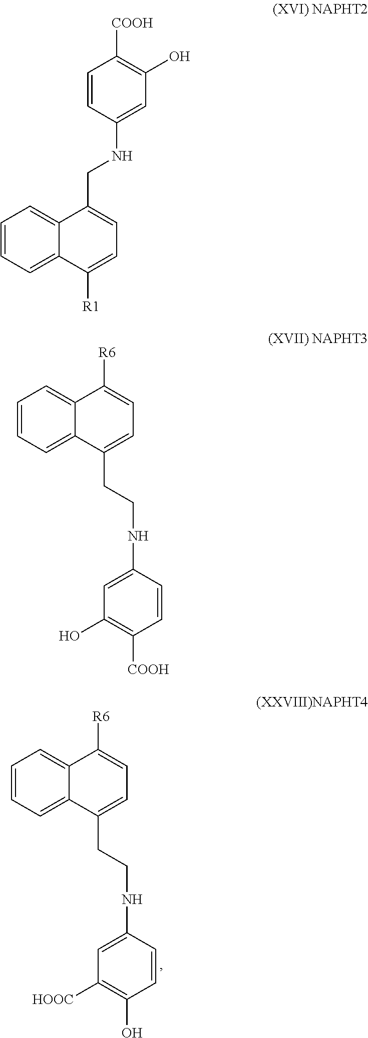

wherein R¹ and R⁶ is a arylcarboxy, C₁-C₈ cycloalkylcarboxy, C₁-C₅ alkylcarboxy, arylcarbamoyl, C₁-C₈ cycloalkylcarbamoyl, C₁-C₅ alkylcarbamoyl group. R¹, R³ and R⁶ are independently a cannabinoid pharmacophore substituent such as arylcarboxy, C₁-C₈ cycloalkylcarboxy, C₁-C₅ alkylcarboxy, arylcarbamoyl, C₁-C₈ cycloalkylcarbamoyl, C₁-C₅ alkylcarbamoyl group. Preferable aryl group derivates include arylalkoxy or arylhalide derivates.

Particularly preferred compounds of the invention, having agonist activity at, at least one of a PPAR and a cannabinoid receptor may be selected from the group consisting of:

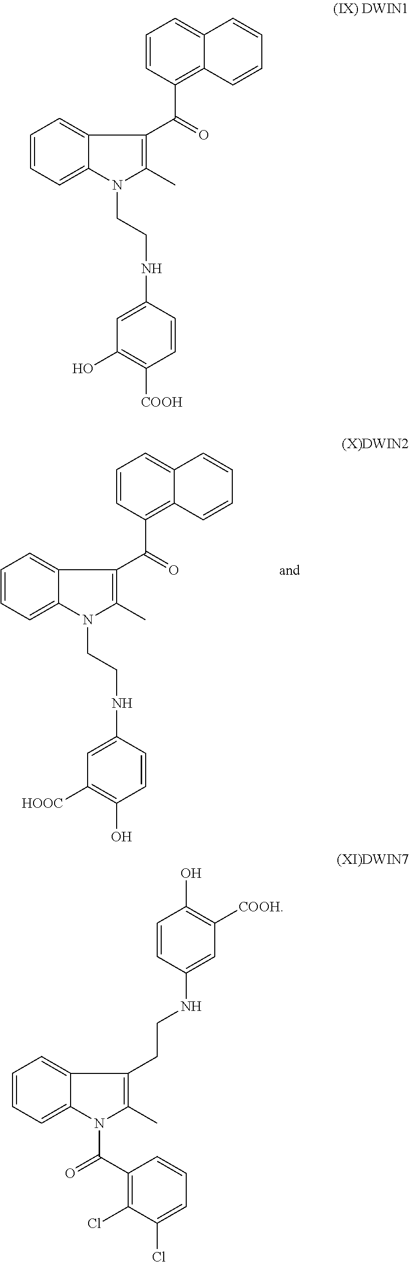

Particularly preferred compounds of the invention, having agonist activity at, at least one of a PPAR and a cannabinoid receptor may be selected from the group consisting of:

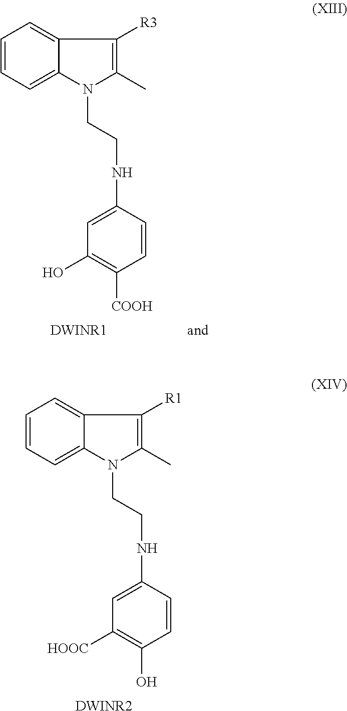

wherein R¹ and R³ is a cannabinoid pharmacophore substituent selected from the group consisting of: a arylcarboxy, C₁-C₈ cycloalkylcarboxy, C₁-C₅ alkylcarboxy, arylcarbamoyl, C₁-C₈ cycloalkylcarbamoyl and C₁-C₅ alkylcarbamoyl group. In a further preferred embodiments, R¹ and R³ is may be arylcarboxy, C₁-C₈ cycloalkylcarboxy, C₁-C₈ alkylcarboxy, arylcarbamoyl, C₁-C₈ cycloalkylcarbamoyl, C₁-C₈ alkylcarbamoyl groups.

Particularly preferred compounds of the invention, having agonist activity at, at least one of a PPAR and a cannabinoid receptor may be selected from the group consisting of:

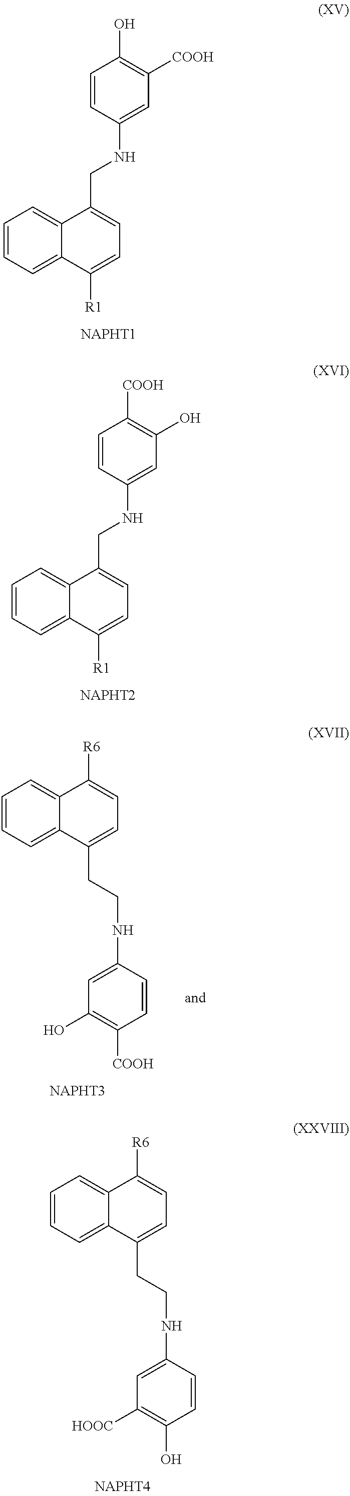

wherein R₁ and R₆ is a cannabinoid pharmacophore substituent selected from the group comprising a arylcarboxy, C₁-C₈ cycloalkylcarboxy, C₁-C₅ alkylcarboxy, arylcarbamoyl, C₁-C₈ cycloalkylcarbamoyl, C₁-C₅ alkylcarbamoyl group.

Equally preferred compounds having agonist activity at least one of a PPAR and a cannabinoid receptor may be selected from the group consisting of:

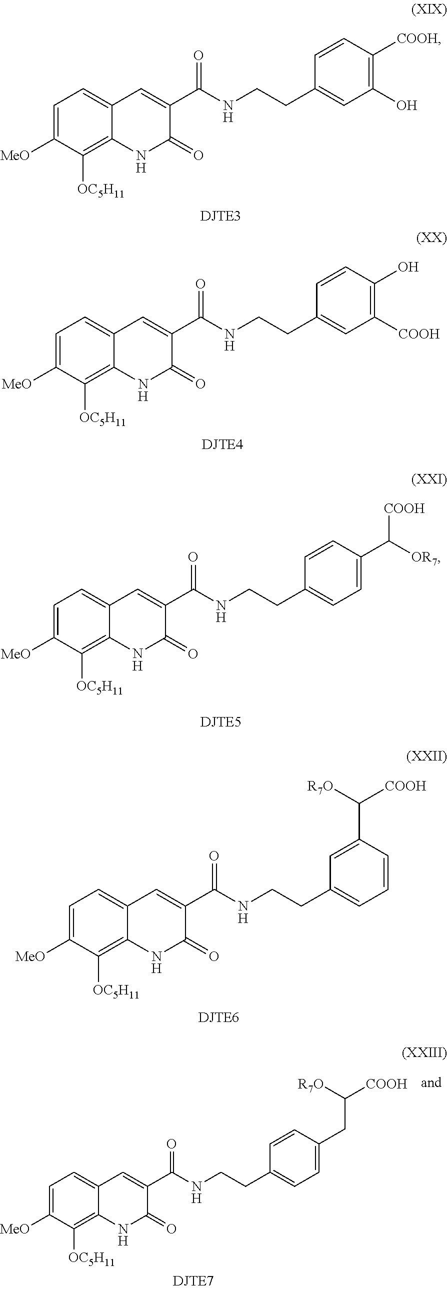

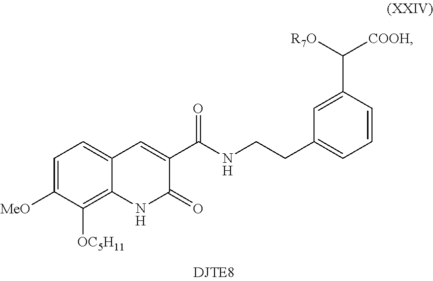

wherein —OR₇ is an alkoxy group such as a C₁-C₅ alkoxyl, a C₃-C₆ cycloalkoxyl group, a vinyloxyl, a C₃-C₅ allyloxyl, benzoxy, naphthaloxy or a benzyloxy group.

Particularly preferred compounds may be selected from the group consisting of:

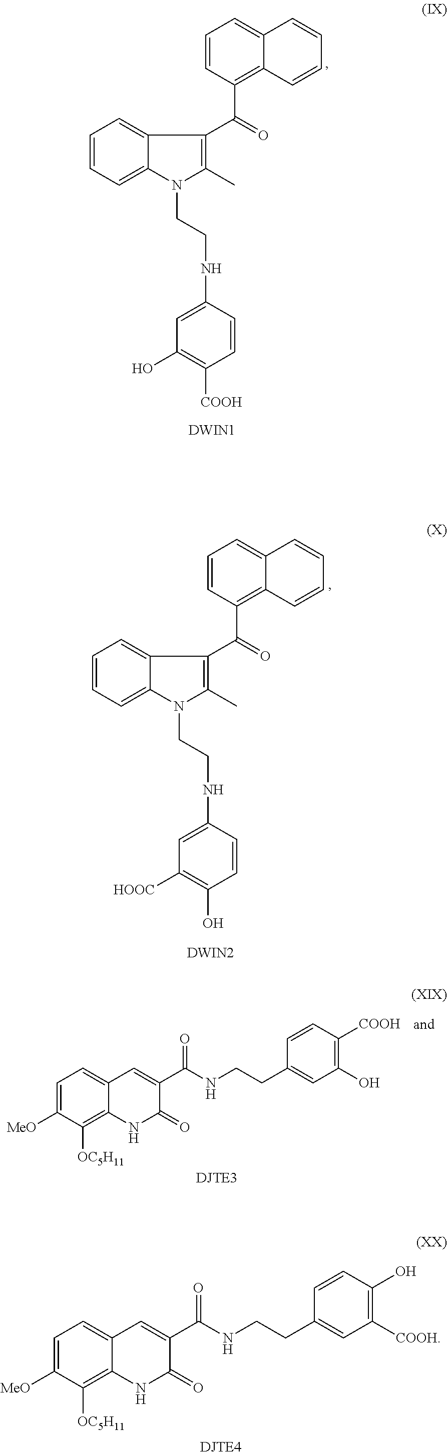

These particular examples are particularly advantageous since they have been shown to be more potent than PPAR-γ agonist control compound GW1929, based on EC50 results provided herein.

Most particularly preferred compounds may be selected from the group consisting of:

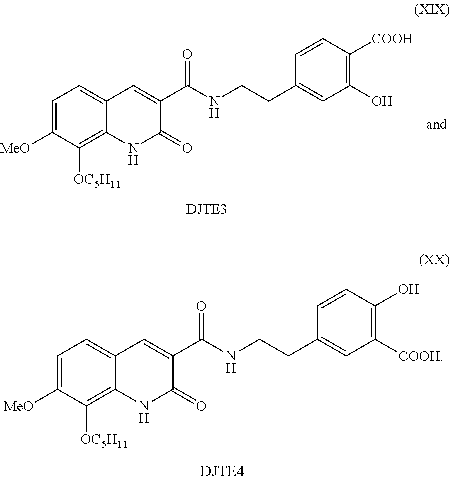

These particular examples are particularly advantageous since they have been shown to have superior potency when compared to PPAR-γ agonist control compound GW1929, based on EC50 results provided herein.

A particularly preferred compound of the invention has structure:

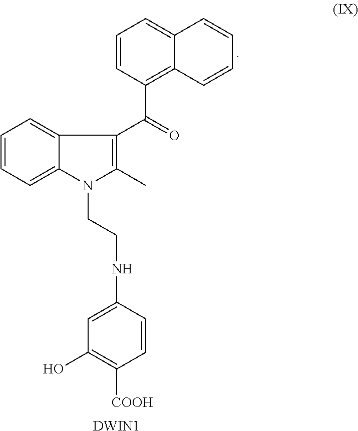

Another particularly preferred compound of the invention has structure:

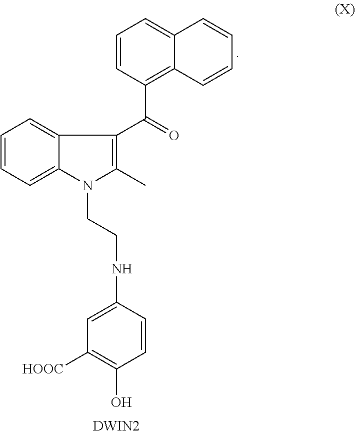

Yet another particularly preferred compound of the invention has structure:

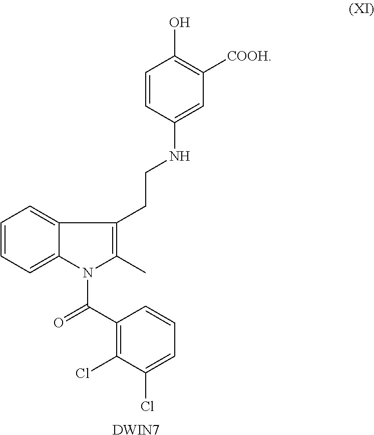

Yet another particularly preferred compound of the invention has structure:

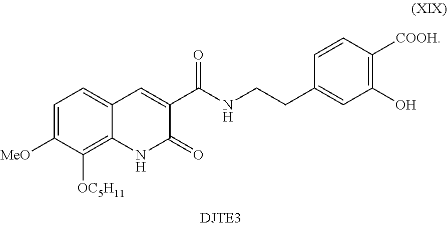

Another particularly preferred compound of the invention has structure:

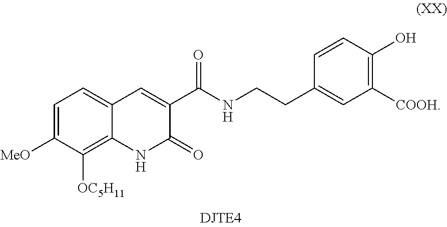

Each of these specific structures are examples of compounds that are at least active at the PPAR-γ receptor. The compounds comprise a cannabinoid pharmacophore as defined by the present invention and thus are expected to also be active at a cannabinoid receptor.

Thus the present invention provides novel MTL compounds, for pharmaceutical compositions containing these compounds and medical and therapeutic uses of such MTL compounds. The compounds of the invention will be active on at least one of the PPARs and at least one of the cannabinoid receptors. The compounds are agonistic at each of the PPAR and cannabinoid receptors.

Thus, the present invention focuses on provision of a series of non-cleavable conjugated MTLs for PPARs and cannabinoid receptors.

In the present invention, compounds which will be active at the PPARs and the cannabinoid receptors have been identified by in silico investigation using 5ASA and 4ASA, but also based on modelling using glitazar, which is known to be a ligand of both PPARα and PPARγ.

Modelled compounds are based on the fact that two compounds displaying activity against different receptors may be linked together by an appropriate cleavable or non-cleavable linker (cleavable or non-cleavable conjugated pharmacophores) or their common pharmacophores may be overlapped (slightly overlapped or highly integrated) (FIG. 1).¹²

Thus the compounds of the invention are designed on the basis of pharmacophore models and in silico virtual screening. The process has resulted in the design of new hybrid molecules that target at least one of a cannabinoid receptor and a peroxisome proliferator-activated receptor, particularly the PPAR-γ receptor and thus the compounds are potentially endowed with anti-inflammatory and neuroprotective actions.

Particularly preferred are compounds having at least one activity but preferably dual agonist activities on both the cannabinoid CB2 receptor 2 (CB2) and the peroxisome proliferator-activated receptor γ (PPAR-γ) receptor.

In general, the compounds of the invention comprise a first part and a part, the first part comprises a PPAR pharmacophore; and the second part comprises a CB pharmacophore, wherein the first and second parts are connected by at least one linker characterized in that the compound is active at, at least one of a PPARs and a CB receptor. The most preferred compounds have dual activities at both the PPARs and CB receptor.

Advantageously, all of the compounds herein are expected to be active to some degree on at least one of PPARα and PPARγ receptors, since there is only one residue differing α (Tyr) and γ (His) active site. α selectivity can be generally achieved by introducing a gem-dimethyl group at the alpha position of the carboxylate as shown in fibrates.

To design compounds with dual activities, knowledge of the structure—activity relationships (SAR) and the pharmacophore requirements for the two target activities was required. This was obtained from (i) literature data and (ii) from docking studies of known CB₂ and PPARγ selective agonist compounds. The data was used to refine three-dimensional models of their respective receptors, which allowed identification of the receptors residues and the compound functional groups, implicated in the molecular recognition process.

Typically, the compounds described herein present a docking scoring value, calculated with the Goldscore fitness function, which is greater than that of WIN-55212-2 or JTE-907 for the CB₂ receptor or greater than the score of 5-ASA for PPAR γ.

The most preferred compounds will have receptor potencies greater than that of PPAR control compound GW1929 in cell free pharmacological activity tests.

The most preferred compounds will have receptor potencies greater than that of PPAR control compound rosoglitazone in cell based pharmacological activity tests.

The compounds described herein can be advantageously used in the design of dual active ligands, active at PPAR and cannabinoid receptors. Further modification can be made to these compounds to optimize further the receptor activities.

In another aspect of the invention the compounds have activity at, at least one of a PPAR and a cannabinoid receptor, particularly a PPAR receptor. Particularly preferred are those compounds, which have activity at a PPAR receptor. The most preferred compounds of this aspect have activity at a PPAR-γ receptor. Particularly preferred compounds in this regard may be selected from the group consisting of:

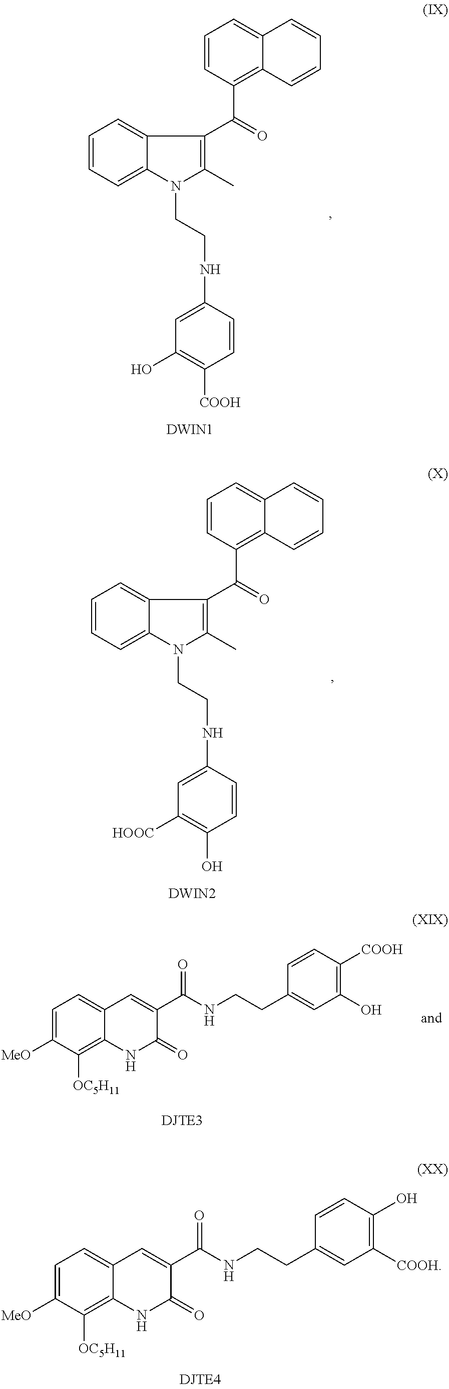

These particular examples are particularly advantageous since they have been shown to be more potent than PPAR-γ agonist control compound GW1929, based on EC50 results provided herein.

Most particularly preferred compounds may be selected from the group consisting of:

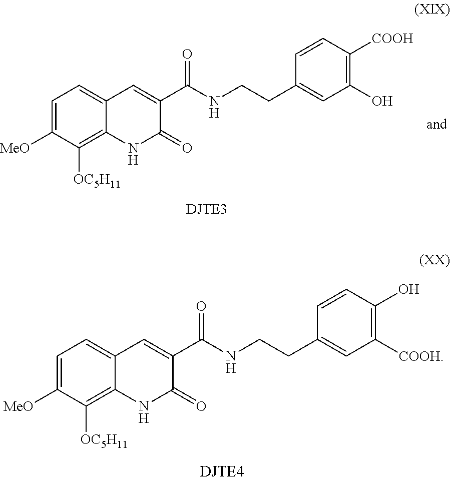

These particular examples are particularly advantageous since they have been shown to have superior potency when compared to PPAR-γ agonist control compound GW1929, based on EC50 results provided herein.

The compounds according to the invention will be used advantageously in the medical field.

Therefore, the present invention further relates to a pharmaceutical composition comprising one or more compounds according to the invention as active principles in combination with one or more pharmaceutically acceptable excipients or adjuvants.

Furthermore, in one aspect, the present invention relates to the use of the compounds according to the invention for the preparation of a medicinal product for the prevention and treatment of conditions involving PPAR, e.g., tumours expressing PPARγ.

In a second aspect, the invention relates to the use of the compounds according to the invention for the preparation of a medicinal product for the prevention and treatment of conditions involving tumours expressing the PPARs.

In a third aspect, the invention relates to the use of the compounds according to the invention for the preparation of a medicinal product for the prevention and treatment of chronic inflammatory diseases. Typically such conditions include irritable bowel disease, Crohn's disease and ulcerative rectocolitis.

The compounds may also be used in the intervention of gastrointestinal tract conditions such as Crohn's disease, ulcerative colitis, intestinal bowel syndrome and acute diverticulitis. In one aspect of the invention, there are provided compounds for use in the prevention of conditions such as acute diverticulitis in patients affected by colonic diverticulosis, indeterminate colitis and infectious colitis.

The compounds according to the present invention can be used advantageously in the medical field to stimulate PPAR-γ to mediate cationic antimicrobial peptides (CAMPs) in epithelia and mucosal tissues. CAMPS include defensin and/or cathelicidin. Insofar as the compounds of the invention stimulate production of cationic antimicrobial peptides (CAMPs) expression though mediation of PPAR receptors, the compounds may be used to stimulate the immune system by producing CAMPs such as defensin and cathelidicin in epithelial and mucosal tissues where PPAR are present. Thus, in one embodiment the compounds of the invention may be used to treat irritable bowel syndrome (IBS) or may be used in the manufacture of a medicament for the treatment of irritable bowel syndrome or other conditions where microbial infection is implicated.

Therefore, another aspect of the present invention relates to a pharmaceutical composition comprising one or more compounds as defined above as active principles in combination with one or more pharmaceutically acceptable excipients or adjuvants.

In a further aspect the present invention relates to a pharmaceutical composition comprising a compound according to the present invention, a tautomer thereof, a pharmaceutically acceptable salt thereof, or a hydrate thereof, together with a pharmaceutically acceptable carrier or excipient.

In another aspect, the invention provides compounds for use in the preparation of a medicament for the treatment and prevention of diseases such as Crohn's disease, ulcerative colitis, irritable bowel syndrome (IBS), acute diverticulitis and prevention of conditions such as acute diverticulitis in patients affected by colonic diverticulosis, indeterminate colitis and infectious colitis.

In another aspect, the compounds and compositions of the invention can be used for the preparation of a medicinal product for the treatment of pain.

The compounds of the present invention can be used for the prevention and treatment of conditions and alleviation of symptoms such as those of pain, inflammation, hyperactivation of the immune system including chronic inflammatory diseases, allergic diseases, autoimmune diseases, metabolic disorders and particularly disease with intestinal inflammation including Crohn disease, ulcerative colitis, indeterminate colitis, infections intestinal inflammation, celiac disease, microscopic colitis, irritable bowel syndrome, hepatitis, dermatitis including atopic dermatitis, contact dermatitis, acne, rosacea, Lupus Erythematosus, lichen planus, and Psoriasis, NASH, liver fibrosis, lung inflammation and fibrosis, but also anxiety, emesis, glaucoma, feeding disorders (obesity), movement disorders, diseases of Central Nervous System, such as multiple sclerosis, traumatic brain injury, stroke, Alzheimer's Disease and Peripheral Neuropathies such as traumatic neuropathies, metabolic neuropathies and neuropathic pain, Atherosclerosis, Osteoporosis, alopecia androgenetica and alopecia aerate.

PPAR disfunction has also been implicated in alopecia, including alopecia androgenetica and alopecia aerate. Thus, the compounds of the invention may be used to treat or prevent these conditions.

The compounds and compositions of the invention can be used to treat humans or animals suffering from any of the conditions described herein.

In the case of activity at the PPARs, experiments involving cells transfected with the PPARs, the quantification of target genes from said infected cells, investigation of the ability of the molecules to induce PPAR translocation into the nucleus and competition-binding assays will allow evaluation of the activity of the compounds. Competition binding assay studies will be useful for investigation into the activity of the compounds at the cannabinoid receptors.

### BRIEF DESCRIPTION OF THE DRAWINGS

The invention will be more clearly understood from the following description of an embodiment thereof, given by way of example only, with reference to the accompanying drawings, in which:—

FIG. 1: Typical Types of Rationally Designed Multi Target Ligands

FIG. 2: Interactions of 5ASA into the PPARγ active site

FIG. 3: Interactions of 4ASA into the PPARα active site

FIG. 4: Interactions of Win-55212-2 into the CB₂ active site

FIG. 5: Interactions of JTE-907 into the CB₂ active site

FIG. 6: Docking of DWIN and DJTE type compounds possessing the 4-ASA feature into the PPARγ active site

FIG. 7: Docking of DWIN and DJTE type compounds possessing the 5-ASA feature into the PPARγ active site

FIG. 8: Docking of DWIN and DJTE type compounds possessing the 4-ASA feature into the PPARα active site

FIG. 9: Docking of DWIN and DJTE type compounds possessing the 5-ASA feature into the PPARα active site

FIG. 10: Docking of DWIN type compounds into the CB₂ active site

FIG. 11: Docking of DJTE compounds into the CB₂ active site

FIG. 12: Activity of a number of compounds of the invention at the PPAR-γ receptor in cell free system (AlphaScreen) versus GW1929 control—test 1.

FIG. 13: Activity of a number of compounds of the invention at the PPAR-γ receptor in cell free system (GeneBlazer) versus GW1929 control—test 2.

FIG. 14: Activity of a number of compounds of the invention at the PPAR-γ receptor in cell based system (GeneBlazer) versus rosglitazone control.

FIG. 15: Activity of WIN 55212-2 control compound at the CB2 receptor in cell based system (GeneBlazer).

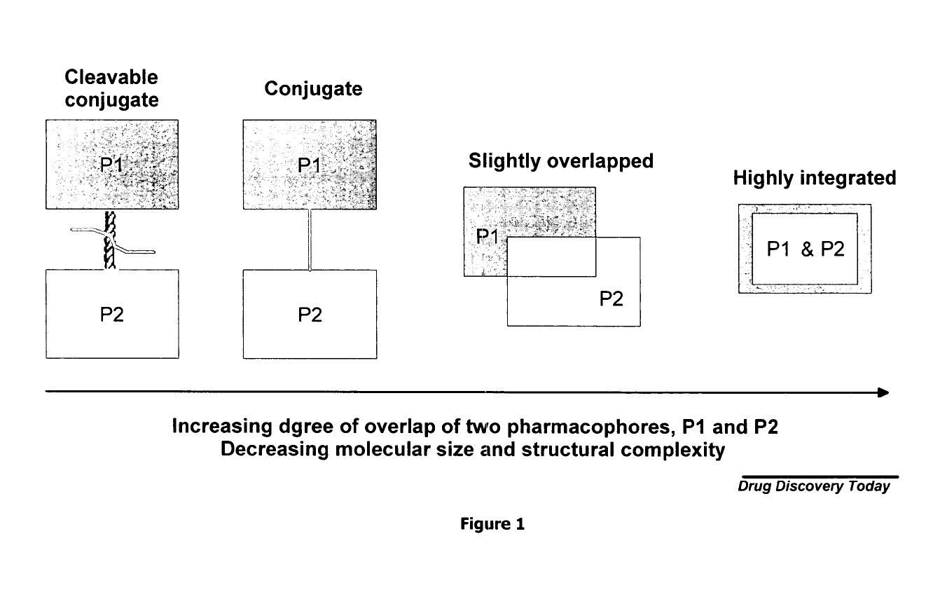

### DETAILED DESCRIPTION OF THE INVENTION

During the course of the studies into the dual active compounds of the present invention of the MTL approach, it was surprising discovered that a number of the compounds have surprisingly advantageous utility at, at least a single receptor, rather than a balanced activity at both receptors concurrently. In particular, it was surprisingly found that a number of the compounds of the invention were particularly potent at a PPAR receptor when compared to normal control compounds known to have reasonable activity for a particular given dose. These compounds when used at comparable doses appear to be substantially more potent at PPAR-γ receptors in particular. The results show that the compounds were surprisingly more active at the PPAR receptor than was initially indicated by the Goldscore docking results initially carried out.

### Design of New Chemical Entities

Compound structural modifications involved introducing the 4-amino (4-ASA) or 5-aminosalicylate (5-ASA) groups, which were known to activate the PPARα and γ receptor, into the CB₂ agonists ligands.

### Non-Cleavable Conjugated Pharmacophores

The compound WIN 55, 212-2 is an example of a potent non-classical cannabinoid receptor agonist, and acts as a potent analgesic in a rat model of neuropathic pain. WIN 55, 212-2 is a member of the aminoalkylindole family and is a weaker partial agonist than THC, but displays a higher affinity towards the CB₁ receptor.

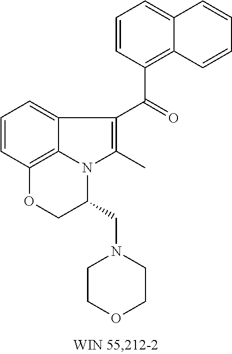

Another compound, JTE-907, a 2-oxoquinoline family member, has been found to be a highly selective CB₂ ligand which behaves as an inverse agonist in vitro, but has an anti-inflammatory effect in vivo.

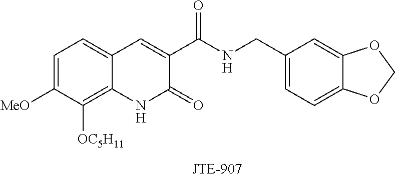

It is known to possess a potent analgesic and anti-inflammatory activity and does not exhibit undesirable psychotropic effects. JTE-907 binds in vitro with high affinity at human CB₁ and CB₂ receptors and exerts an agonist activity. Moreover, AJA binds to PPARγ and activates the receptor. Its anti-inflammatory activity is certainly mediated by this mechanism.^{8,21,22}

Thus aminoalkylindoles and 2-oxoquinolines were chosen as starting points in the design of non-cleavable conjugated pharmacophores.

In the aminoalkylindoles family, the morpholine group of WIN-55212-2 derivatives was replaced by the 4-amino (4-ASA) or 5-aminosalicylate (5-ASA) group.

SAR data indicated that exchange at the R₁ and R₃ substituents on the aminoalkylindole should lead to retention of target activity.¹⁹

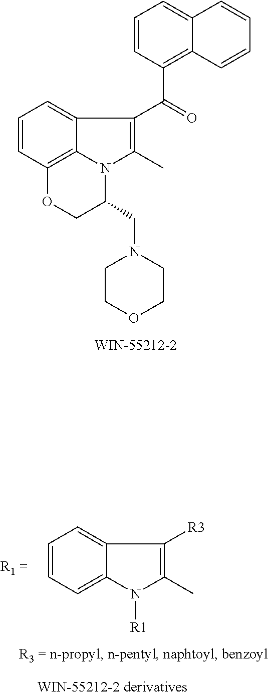

In the 2-oxoquinoline family, the benzodioxole group of JTE-907 was replaced by salicylate groups.^{19,20}

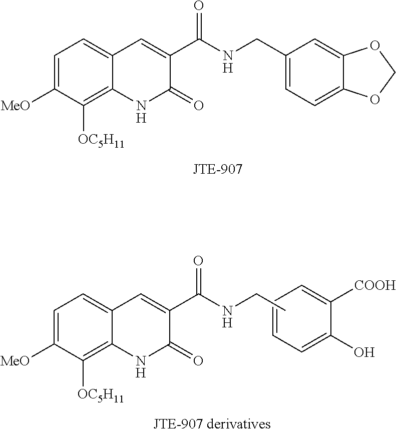

The structure of the human PPARs ligand-binding domain was obtained from its complexed tesaglitazar (AZ 242) X-Ray crystal structure which is available in the RCSB Protein Data Bank (http://www.rcsb.org/pdb/home/home.do) (PDB ID: 117I).^{16,17}

Since the experimental determination of the G-protein coupled receptors (GPCRs) structures has not yet been realised, a theoretical model of the CB₂ receptor was constructed by homology modelling using the X-ray structure of the GPCR bovine rhodopsin as a template.¹⁸

Structurally modified CB₂ selective agonist compounds and their PPARs and CB₂ active sites binding modes were investigated (see Tables 1 and 2). The retained compounds were found to belong to the classical and non-classical cannabinoids, i.e., the aminoalkylindoles and 2-oxoquinolines families respectively.

### Molecular Modelling

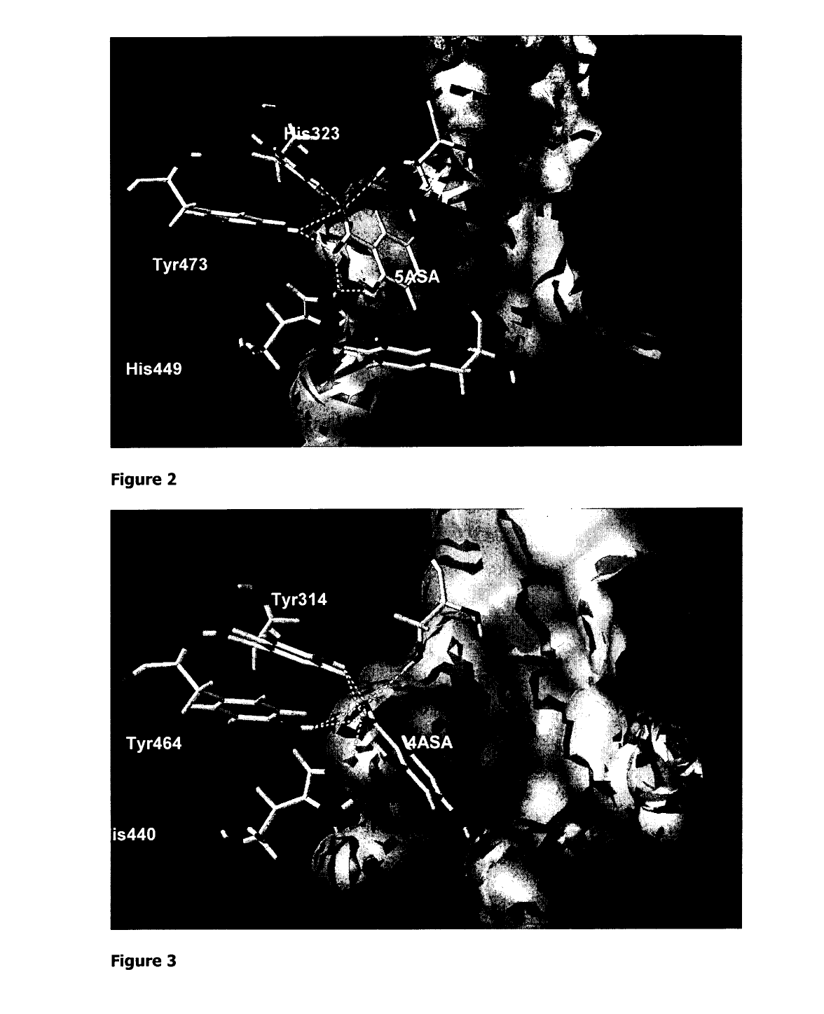

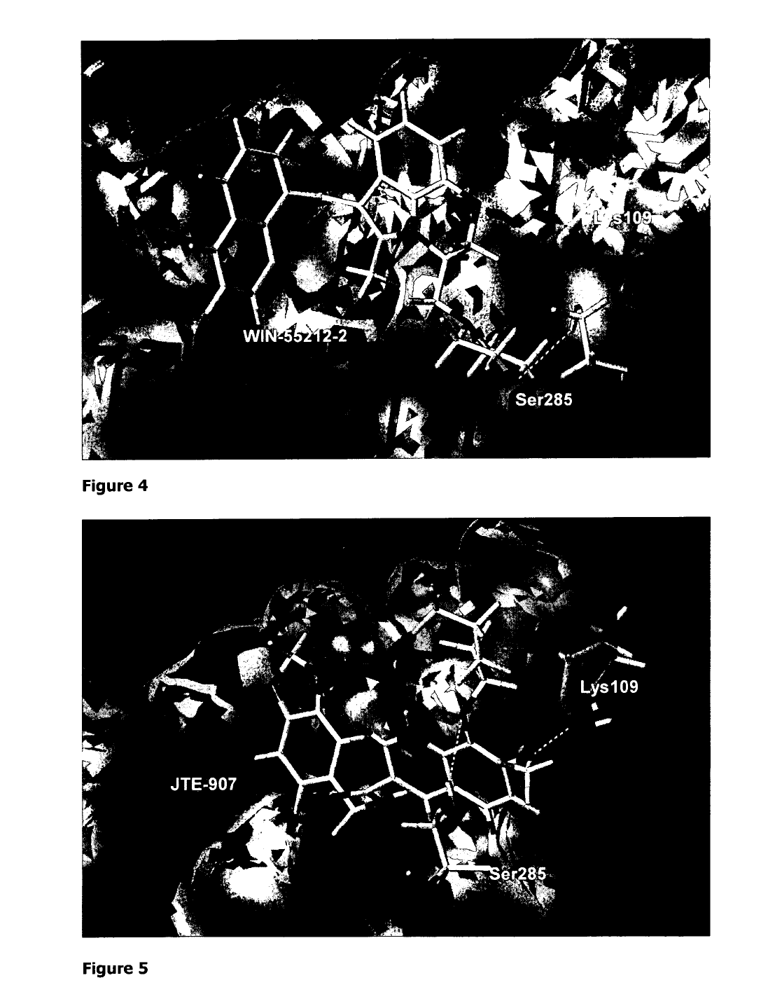

Docking simulations were carried out in order to predict the binding mode of these compounds in the PPARs and CB₂ active sites. Automated docking of the ligands into the receptors active sites provided multiple docking solutions. Among the best scored solutions, a visual inspection was performed to retain the conformations forming the interactions considered to be essential for the PPARγ activity, including hydrogen bonding with His323, His449, and Tyr473 (FIG. 2), those for the PPARα activity, including hydrogen bonding with Tyr314, His440, and Tyr464 (FIG. 3), and also those for the CB₂ agonist activity, i.e., multiple hydrophobic contacts and hydrogen bonding with Lys109 and/or Ser285 (FIGS. 4 and 5).

### Materials and Methods

Molecular modelling studies were performed using SYBYL software version 6.9.1²⁵ running on Silicon Graphics Octane 2 workstations. As the pkₐ of compounds are unknown, the SPARC online calculator was used to determine the species occurring at physiological pH (7.4) (http://ibmlc2.chem.uga.edu/sparc/index.cfm)²⁶. Three-dimensional model of ionized compounds were built from a standard fragments library, and their geometry was subsequently optimized using the Tripos force field²⁷ including the electrostatic term calculated from Gasteiger and Hückel atomic charges. The method of Powell available in the Maximin2 procedure was used for energy minimization until the gradient value was smaller than 0.001 kcal/mol.Å. The structure of the human PPARs ligand-binding domain was obtained from its complexed X-Ray crystal structure with the tesaglitazar (AZ 242) available in the RCSB Protein Data Bank (http://www.rcsb.org/pdb/home/home.do)¹⁷ (PDB ID: 117I)^{16,17}. An homology model of the CB₂ receptor was constructed by aligning its sequence (UniProtKB entry: P34972)²⁸ on the bovine rhodopsine (UniProtKB entry: PO2699)²⁹ with ClustalW³⁰ then transferring the 3D coordinates of the bovine rhodopsine crystallographic structure (PDB ID: 1U19)³¹ with Jackal.³² In order to create a model in a putative activated conformation, transmembrane domains 3 and 6 (TM3 and TM6) were rotated by 20° and 30° respectively as described for CB₁ by McAllister and coworkers.³³ Flexible docking of the compounds into the receptors active sites was performed using GOLD 3.1.1 software. The most stable docking models were selected according to the best scored conformation predicted by the GoldScore scoring function.³⁴ The complexes were energy-minimized using the Powell method available in Maximin2 procedure with the Tripos force field and a dielectric constant of 4.0 until the gradient value reached 0.01 kcal/mol.Å. The anneal function was used to define a 10 Å hot region and a 15 Å region of interest around the ligand.

### Results

The best docking results for both PPARs and CB₂ receptors were obtained with pharmacophores derivatives, according to their GoldScore values (Tables 1 and 2). The GoldScore fitness function has been optimised for the prediction of ligand binding positions and takes into account factors such as H-bonding energy, van der Waals energy and ligand torsion strain. GoldScore give fitness scores that are dimensionless however, the scale of the score gives a guide to how good the pose is; the higher the score, the better the docking result is likely to be. GoldScore represents strength of binding interaction.

Results for examples of WIN-55212-2 derivatives (DWIN) and JTE-907 derivatives (DJTE) are presented in Tables 1 and 2 respectively.

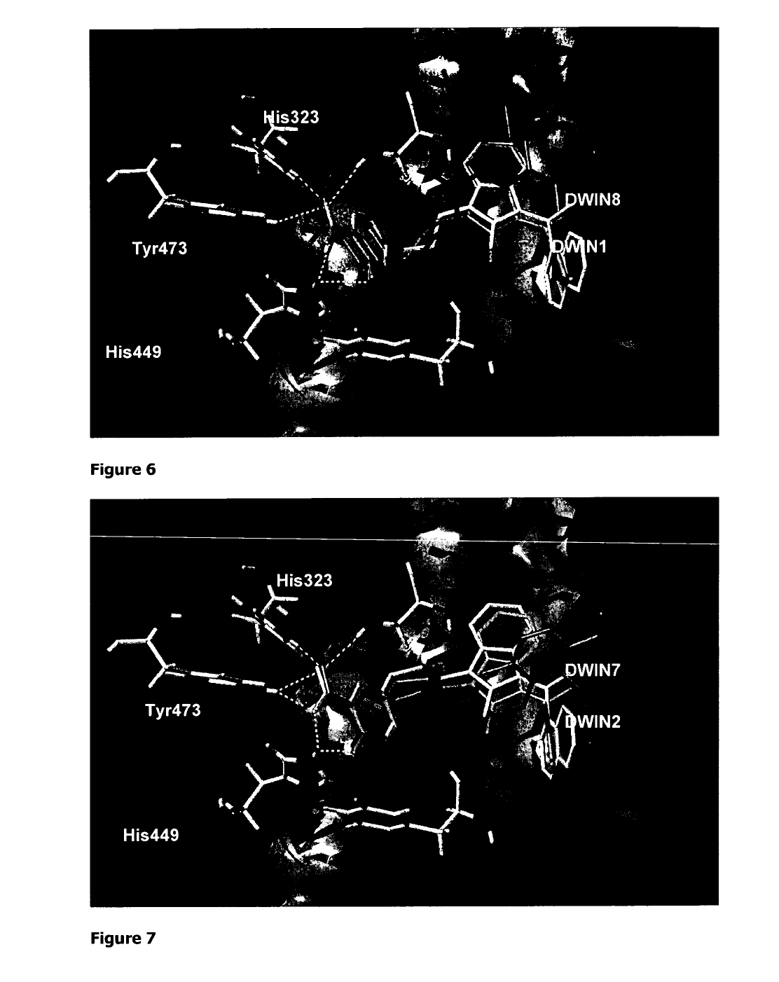

Docking results of DWIN and DJTE compounds into the PPARγ active site are presented in FIGS. 6 and 7.

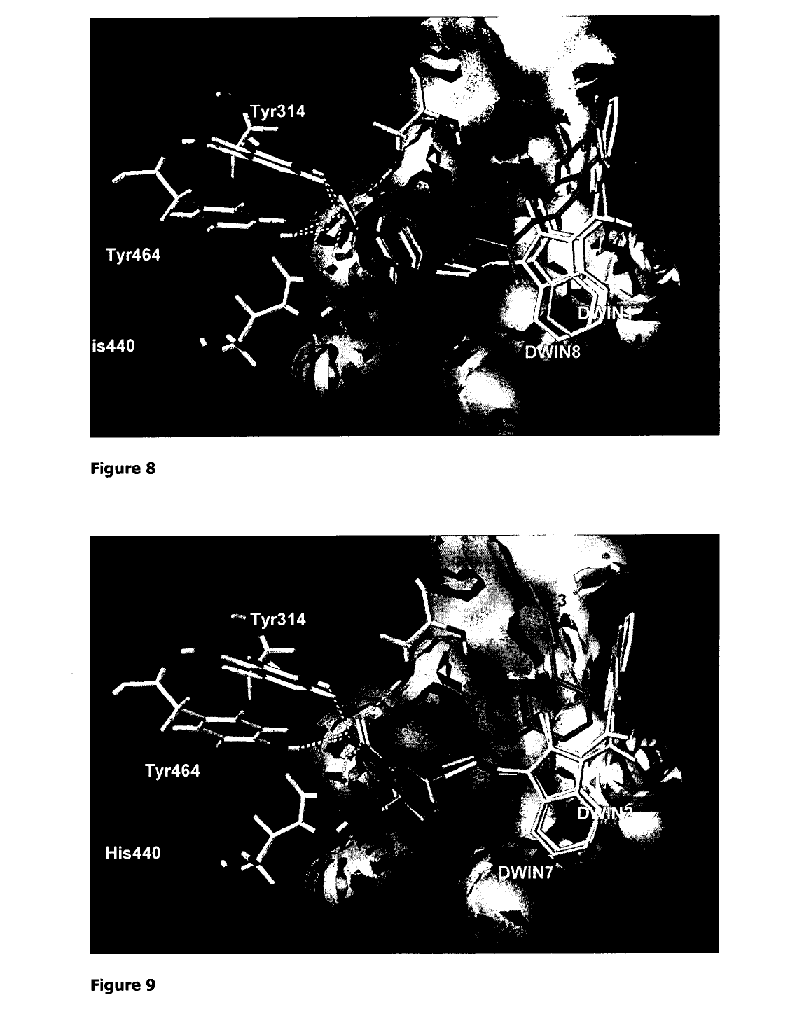

Docking results of DWIN and DJTE compounds into the PPARα active site are presented in FIGS. 8 and 9.

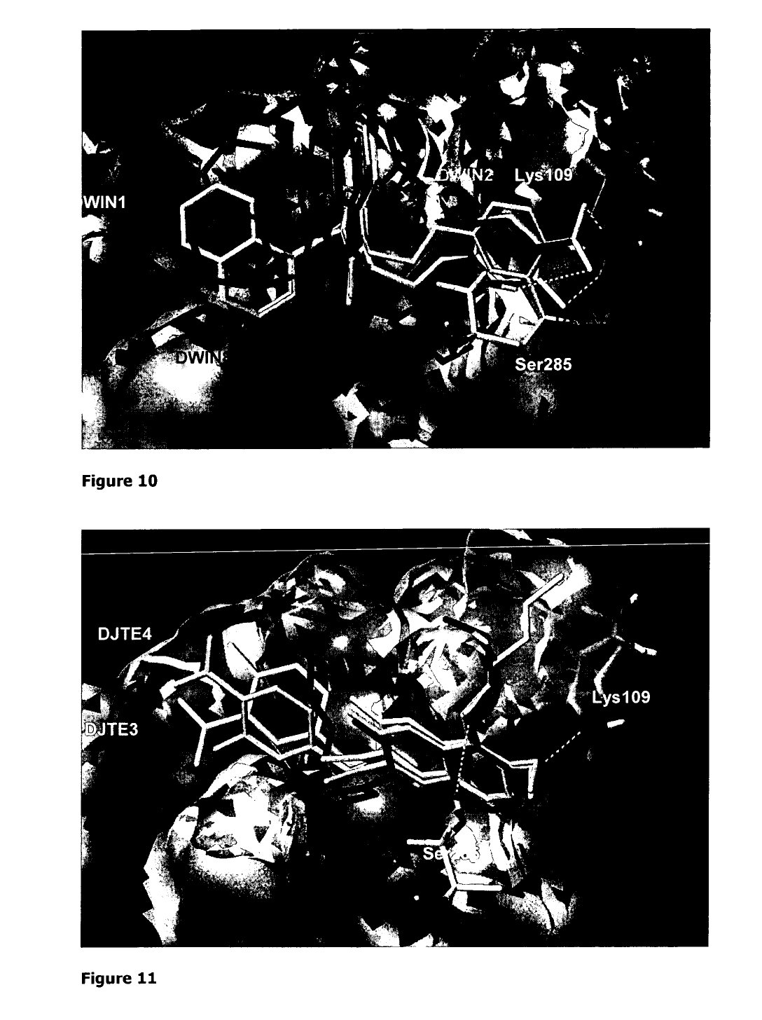

Docking results of DWIN and DJTE compounds into the CB₂ active site are presented in FIGS. 10 and 11 respectively. Generally speaking, the new designed compounds scoring values are higher than reference ligands for PPARγ (4-ASA, 5-ASA) and are in the same range for CB₂ (WIN-55212-2, JTE-907).

TABLE 1
Docking results for some WIN-55212-2 derivatives.

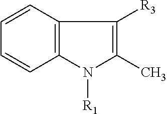

| | | | GoldScore | GoldScore | GoldScore |
|---|---|---|---|---|---|
| Compounds | R₁ | R₃ | PPARα | PPARγ | CB₂ |
| DWIN1 (IX) | 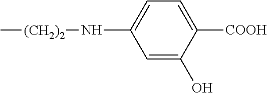 | 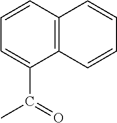 | 64.93 | 75.37 | 49.29 |
| DWIN2 (X) | 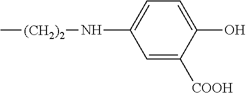 | 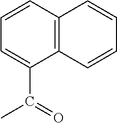 | 64.56 | 71.72 | 42.20 |
| DWIN7 (XI) | 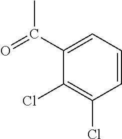 | 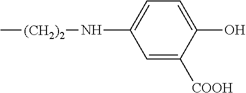 | 56.02 | 67.40 | 40.34 |
| DWIN8 (XII) | 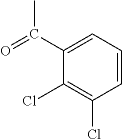 | 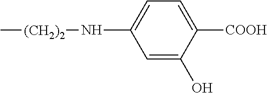 | 54.88 | 67.30 | 50.48 |
| 4-ASA | | | 41.76 | 34.83 | — |
| 5-ASA | | | 44.31 | 34.27 | — |
| WIN-552122 | | | — | — | 50.13 |

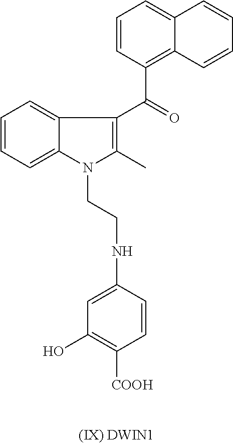

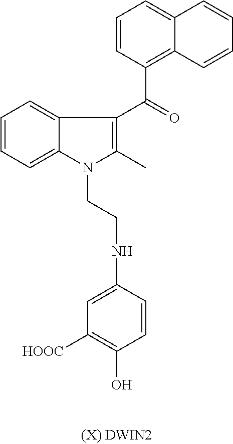

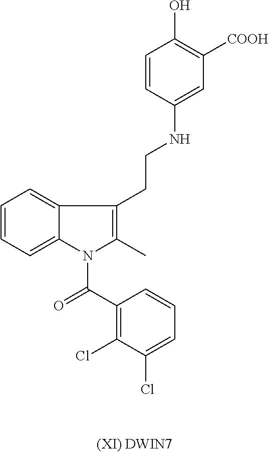

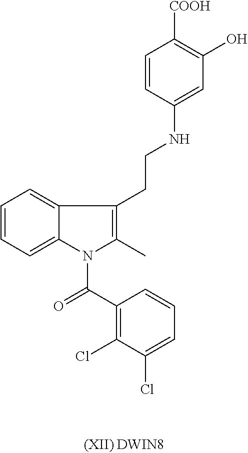

The GoldScore fitness function reflects the theoretical energy necessary to the position the ligand in the ligand binding domain of the receptor. It has been optimised for the prediction of ligand binding positions rather than the prediction of binding affinities, although some correlation with the latter has been found. It was designed to discriminate between different binding modes of the same molecule. Extra terms are probably required to compare different molecules. For example, a term is probably required to account for the entropic loss associated with freezing rotatable bonds when the ligand binds.

TABLE 2
Docking results for some JTE-907 derivatives.

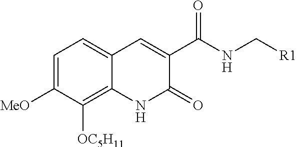

| | | | | Gold- |
|---|---|---|---|---|
| Com- | | GoldScore | GoldScore | Score |
| pounds | R₁ | PPARα | PPARγ | CB₂ |
| DJTE3 (XIX) | 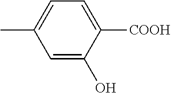 | 69.73 | 69.13 | 40.33 |
| DJTE4 (XX) | 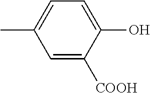 | 66.72 | 73.33 | 39.17 |
| 4-ASA | | 41.76 | 34.83 | — |
| 5-ASA | | 44.31 | 34.27 | — |
| JTE-907 | | — | — | 41.21 |

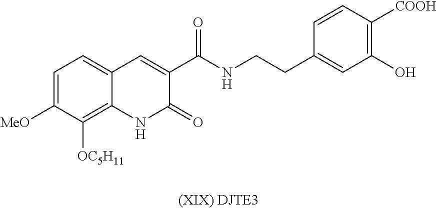

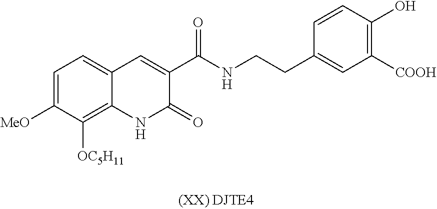

It is expected that molecules having the best Goldscores for PPARγ and CB₂ will have a synergistic anti-inflammatory and analgesic effect mediated by PPARs and CB₂. The preferred compounds of the invention are those having docking Goldscore greater than that of WIN-55212-2 or JTE-907 for the CB receptor or greater than the score of 5-ASA for PPAR γ receptor.

### Conclusion

The highest ranking compounds, indicated from modelling studies, all show an activity similar/superior to that of mesalazine and JTE-907.

All chemically feasible variations were evaluated in order to achieve the best score (affinity and activation of the receptor) in computer docking experiments. Consequently, it is believed that the compounds of the present invention show comparable function and/or activity to mesalazine and AJA and do so through similar biological pathways.

### Synthesis of Chemical Compounds

### General

Commercial chemicals were purchased from Aldrich unless stated otherwise and were used as received. Flash column chromatography was carried out using Merck silica gel 60 (0.040-0.063 mm). Thin layer chromatography was performed on pre-coated plastic plates (Merck silica 60F254), and visualised using UV light and were developed with either aqueous KMnO4 or cerric ammonium molybdate (CAM). Proton (1H) and carbon (13C) NMR spectra were recorded on Varian INOVA 300, 400 and 500 spectrometers. Chemical shifts are quoted relative to tetramethylsilane and referenced to residual solvent peaks as appropriate. Infrared spectra were recorded on a Varian 3100 FT-IR Excalibur Series spectrophotometer as neat liquids or evaporated films using NaCl plates. LR-MS were acquired using a Waters Separations Module linked to a Micromass Quattro micro electrospray mass spectrometer. HPLC analysis was performed using a Thermo Separation Products system (Chromsoft software) with 20 μl injections.

DJTE3 and DJTE4

### Synthesis of Intermediate Acid 5 for DJTE3 and DJTE4

Intermediate 5 was prepared using the literature procedure of Raitio et al. [1] and the yields and spectroscopic data for compounds 1, 2, 3, 4 and 5 were consistent with the data given in this reference.

### Synthesis of DJTE3:2-Hydroxy-5-{[(7-methoxy-2-oxo-8-pentyloxy-1,2-dihydroquinoline-3-carbonyl)-amino]-methyl}-benzoic acid methyl ester 6

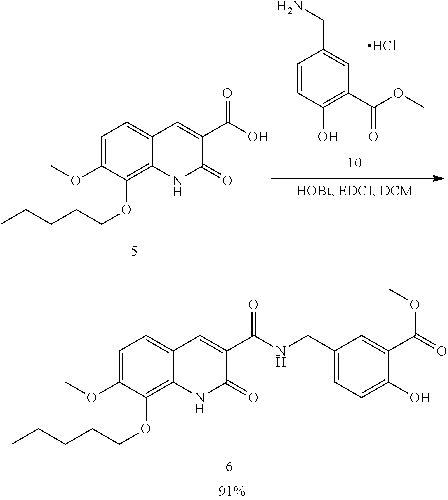

Acid 5 (0.4 g, 1.31 mmol, 1 eq), 5-aminomethyl salicylic acid methyl ester HCl (0.26 g, 1.43 mmol, 1.095 eq), 1-hydroxybenzotriazole (0.196 g, 1.44 mmol, 1.102 eq) and N-(3-dimethylaminopropyl)-N′-ethylcarbodiimide.HCl (0.276 g, 2.176 mmol, 1.66 eq) were dissolved in DCM (2 ml) and were stirred at ambient temperature for 18 h. The reaction mixture was poured into water (10 ml)

and DCM (10 ml) was added, the pH was adjusted to 7 with dil. aq. NaOH and the organic layer was poured off. The aqueous layer was then extracted with DCM (2×10 ml) and the combined organic layers were washed with water (2×10 ml), were washed with brine (10 ml), were dried over Na2SO4, filtered and the
solvent was removed in vacuo. The product was purified via column chromatography eluted with a gradient from 1:1 to 1:3 CyH:EtOAc (Rf product=0.7, Rf acid 5=0.4 in DCM/5% MeOH, UV, CAM). This gave 0.558 g (91%) of the product as a white solid. 1H-NMR (CDCl3) 500 MHz: δ (ppm)=0.94 (3H, t, J=7.1 Hz, CH2CH2CH3), 1.35-1.50 (4H, m, CH2CH2CH3), 1.81 (2H, quin, J=7.8 Hz, CH2CH2CH2CH3), 3.93 (3H, s, COOCH3), 3.97 (3H, s, COCH3), 4.13 (2H, t, J=6.9 Hz, OCH2CH2), 4.58 (2H, d, J=5.9 Hz, NHCH2C), 6.93 (1H, d, J=8.9 Hz, CHCOCH3), 6.95 (1H, d, J=8.8 Hz, CHCOH), 7.45 (1H, d, J=8.5 Hz, CHCHCOCH3), 7.50 (1H, dd, J=2.3 Hz, J=8.5 Hz, CHCHCOH), 7.84 (1H, d, J=2.3 Hz, CHCCOH), 8.90 (1H, s, CCCHCCONH), 9.12 (1H, br.s, CNHCOC), 9.97 (1H, br.t, J=5.5 Hz, NHCH2C), 10.69 (1H, s, COH). 13C-NMR (CDCl3) 125 MHz: δ (ppm)=14.0 (CH3), 22.4 (CH2), 28.0 (CH2), 29.9 (CH2), 42.8 (NCH2), 52.2 (COOCH3), 56.3 (OCH3), 73.8 (OCH2), 109.1 (CH), 112.2 (C), 114.2 (C), 117.9 (CH), 119.4 (C), 125.3 (CH), 129.1 (CH), 129.6 (C), 132.4 (C), 133.5 (C), 135.5 (CH), 145.1 (CH), 154.4 (CH), 160.8 (C), 162.1 (CO), 163.6 (CO), 170.4 (CO). IR Spectrum; evaporated film: v˜(cm-1)=32.45, 29.53, 1672, 1621, 1534, 1495, 1355, 1288, 1213, 1110. MS-ES (negative): 467.7 (M−H+). MS-ES (positive): 469.8 (M+H+). HPLC: 14.615 min.

### 2-Hydroxy-5-{[(7-methoxy-2-oxo-8-pentyloxy-1,2-dihydroquinoline-3-carbonyl)-amino]-methyl}-benzoic acid DJTE3

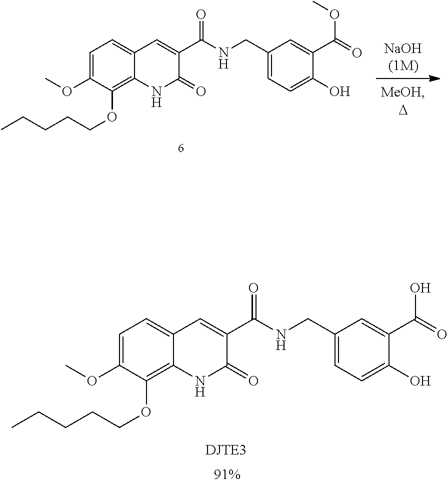

Methylester 6 (0.558 g, 1.19 mmol, 1 eq) and NaOH (0.189 g, 4.72 mmol, 4 eq) was stirred in methanol (15 ml) and water (5 ml) at reflux temperature. Hydrolysis was followed by HPLC (SM=14.615 min, product=10.857 min) and was complete in 3 h. The reaction mixture was then cooled and the pH was adjusted to 4 with dil. aq. HCl, which caused the product to precipitate out of solution as a white solid which was washed with water (20 ml) and ether (20 ml), collected and dried in vacuo to give 0.493 g (91%) of a white powder. 1H-NMR (DMSO D6) 500 MHz: δ (ppm)=0.89 (3H, t, J=7.1 Hz, CH2CH2CH3), 1.35-1.45 (4H, m, CH2CH2CH3), 1.78 (2H, quin, J=7.2 Hz, CH2CH2CH2CH3), 3.93 (3H, s, COCH3), 3.99 (2H, t, J=6.9 Hz, OCH2CH2), 4.50 (2H, d, J=6.0 Hz, NHCH2C), 6.92 (1H, d, J=8.5 Hz, CHCOH), 7.13 (1H, d, J=8.9 Hz, CHCOCH3), 7.50 (1H, dd, J=2.2 Hz, J=8.5 Hz, CHCHCOH), 7.69 (1H, d, J=8.9 Hz, CHCHCOCH3), 7.78 (1H, d, J=2.2 Hz, CHCCOH), 8.79 (1H, s, CCCHCCONH), 10.08 (1H, br.t, J=6.0 Hz, NHCH2C), 11.27 (1H, br.s, COH), 11.51 (1H, s, CNHCOC), 13.77 (1H, br.s, COOH). 13C-NMR (DMSO D6) 125 MHz: δ (ppm)=13.8 (CH3), 21.8 (CH2), 27.3 (CH2), 28.6 (CH2), 41.4 (NCH2), 56.3 (OCH3), 72.7 (OCH2), 109.2 (CH), 113.1 (C), 113.7 (C), 117.0 (CH), 118.7 (C), 125.7 (CH), 129.0 (CH), 130.0 (C), 132.2 (C), 133.9 (C), 134.9 (CH), 144.1 (CH), 154.1 (C), 160.0 (C), 162.1 (CO), 162.9 (CO), 171.6 (CO). IR Spectrum; solid state: v˜(cm-1)=3584, 3325, 3164, 3033, 2930, 2861, 1670, 1593, 1539, 1465, 1333, 1284, 1228, 1113. MS-ES (negative): 453.6 (M−H+). MS-ES (positive): 455.7 (M+H+). HPLC: 10.857 min, >99.1% purity.

Synthesis of DJTE4

### Note on the Synthesis of Acetonide 11 and Bromide 12

The synthesis of these two compounds was undertaken using the procedure of Kang et al.[5] However, changes were made and the actual procedures used are given in elsewhere herein. The spectroscopic data acquired on the products was consistent with the data given by Kang et al.

### 2,2,7-Trimethyl-benzo[1,3]dioxin-4-one 11

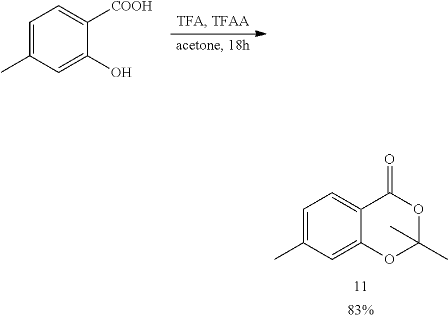

Trifluoroacetic acid (50 ml) and acetone (12 ml) were added to the 4-methysalicylic acid (10 g, 65.72 mmol, 1 eq). Reaction mixture was cooled to 0° C. and trifluoroacetic anhydride (30 ml) was added dropwise over 2 min. Reaction mixture was stirred for 3 days at room temperature and then the volatiles were removed in vacuo. The residues were purified through a dry-flash silica plug eluted with DCM (˜800 ml). The oil was then additionally purified through another dry-flash silica gel plug eluted with toluene (˜1 L). This gave the product as a yellow waxy solid (10.475 g, 83%).

### 7-Bromomethyl-2,2-dimethyl-benzo[1,3]dioxin-4-one 12

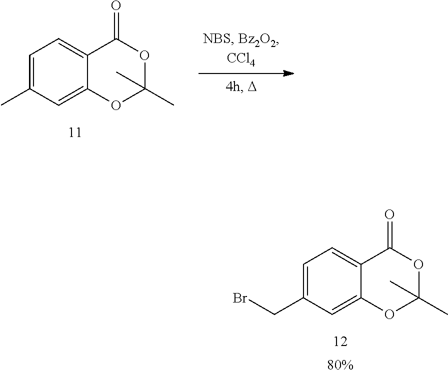

Acetonide 11 (6.0 g, 31 mmol, 1 eq), N-bromo succinimide (6.4 g, 36 mmol, 1.16 eq) and benzoyl peroxide (2.25 g, 7 mmol, 0.22 eq) were dissolved in carbontetrachloride (20 ml). The reaction mixture was stirred at 75° C. for 2 h and was then allowed to cool to ambient temperature. The white precipitate was filtered out and was washed with a small amount of cyclohexane. The filtrate was concentrated in vacuo and the residues were purified via a dry-flash silica gel plug eluted with DCM (˜300 ml). DCM was evaporated. This gave bromide 12 at about 80% conversion by 1H-NMR and this material was used directly in the next step.

### 4-Aminomethyl-2-hydroxy-benzoic acid methyl ester 14

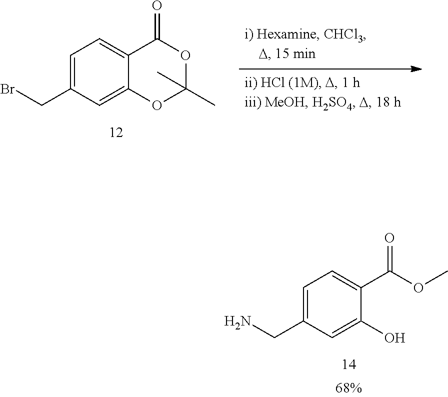

Bromide 12 (0.574 g, 2.12 mmol, 1 eq) was dissolved in chloroform (10 ml), hexamethylenetetramine (0.44 g, 3.18 mmol, 1.5 eq) was added and the mixture was heated to reflux temperature for 15 min. The reaction mixture was cooled and the resulting white solid was removed via filtration and washed with chloroform. This white solid was then heated to reflux in dil. aq. 1M HCl (10 ml) for 1 h. The volatiles were then removed in vacuo and the residues were azeotropically dried with MeOH. The residues were taken up in methanol (20 ml), conc. H2SO4 (3 ml) was added and the mixture was heated to reflux temperature overnight. The reaction mixture was allowed to cool to ambient temperature and was then poured into a separating funnel, water (10 ml) and DCM (50 ml) were added. The layers were shaken and separated and the organic layer was discarded. Then DCM (50 ml) was added and the pH was adjusted to 7 and the organic layer was poured off. The aqueous layer was then extracted with DCM (2×50 ml) and the combined organic layers were washed with water (2×10 ml), were washed with brine (10 ml), were dried over Na2SO4, filtered and the solvent was removed in vacuo. This gave 0.263 g (68%) of an off white solid. 1H-NMR (CDCl3) 500 MHz: δ (ppm)=1.56 (2H, br.s, NH2), 3.85 (2H, s, NCH2C), 3.93 (3H, s, COOCH3), 6.83 (1H, d, J=8.2 Hz, CH2CCHCHC), 6.93 (1H, s, CCHC), 7.78 (1H, d, J=8.2 Hz, CH2CCHCHC), 10.72 (1H, br.s, COH). 13C-NMR (CDCl3) 125 MHz: δ (ppm)=46.2 (CH2), 52.3 (CH3), 110.8 (C), 115.4 (CH), 117.9 (CH), 130.1 (CH), 151.9 (C), 161.8 (C), 170.4 (CO). IR Spectrum; evaporated film: v˜(cm-1)=3585, 3288, 3170, 2960, 1675, 1622, 1575, 1441, 1341, 1259, 1092. MS-ES (negative): 180.1 (M−H+). MS-ES (positive): 182.1 (M+H+).

### 2-Hydroxy-4-{[(7-methoxy-2-oxo-8-pentyloxy-1,2-dihydroquinoline-3-carbonyl)-amino]-methyl}-benzoic acid methyl ester 7

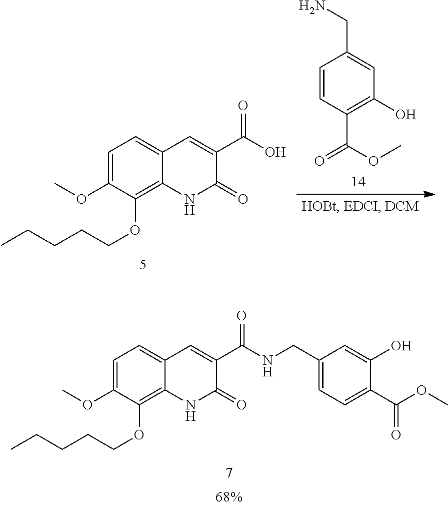

Prepared on 0.328 mmol scale using the same procedure as for 7 (Section 5.4.1). The product was purified via column chromatography eluted with a gradient from 1:1 to 1:3 CyH:EtOAc (Rf product=0.7, Rf acid 5=0.4 in DCM/5% MeOH, UV, CAM). This gave 0.341 g (68%) of the product as a white solid. 1H-NMR (CDCl3) 500 MHz: δ (ppm)=0.94 (3H, t, J=7.1 Hz, CH2CH2CH3), 1.35-1.50 (4H, m, CH2CH2CH3), 1.82 (2H, quin, J=7.7 Hz, CH2CH2CH2CH3), 3.93 (3H, s, COOCH3), 3.98 (3H, s, COCH3), 4.14 (2H, t, J=6.9 Hz, OCH2CH2), 4.67 (2H, d, J=6.0 Hz, NHCH2C), 6.88 (1H, d, J=8.2 Hz, NHCH2CCHCHC), 6.94 (1H, d, J=8.9 Hz, CHCHCOCH3), 6.99 (1H, s, CCHCOH), 7.45 (1H, d, J=8.9 Hz, CHCHCOCH3), 7.78 (1H, d, J=8.2 Hz, NHCH2CCHCHC), 8.89 (1H, s, CCCHCCONH), 9.15 (1H, br.s, CNHCOC), 10.06 (1H, br.t, J=5.7 Hz, NHCH2C), 10.72 (1H, s, COH). 13C-NMR (CDCl3) 125 MHz: δ (ppm)=14.0 (CH3), 22.4 (CH2), 28.0 (CH2), 29.9 (CH2), 43.0 (NCH2), 52.2 (COOCH3), 56.3 (OCH3), 73.9 (OCH2), 109.1 (CH), 111.2 (C), 114.3 (C), 116.0 (CH), 118.2 (CH), 119.3 (C), 125.2 (CH), 130.2 (CH), 132.4 (C), 133.5 (C), 145.2 (CH), 147.3 (C), 154.4 (C), 161.8 (C), 162.1 (CO), 163.8 (CO), 170.4 (CO). IR Spectrum; evaporated film: v˜(cm-1)=3242, 3189, 2954, 2864, 1671, 1622, 1534, 1342, 1260, 1214, 1110. MS-ES (negative): 467.2 (M−H+). MS-ES (positive): 469.3 (M+H+). HPLC: 14.730 min.

### 2-Hydroxy-4-{[(7-methoxy-2-oxo-8-pentyloxy-1,2-dihydroquinoline-3-carbonyl)-amino]-methyl}-benzoic acid DJTE4

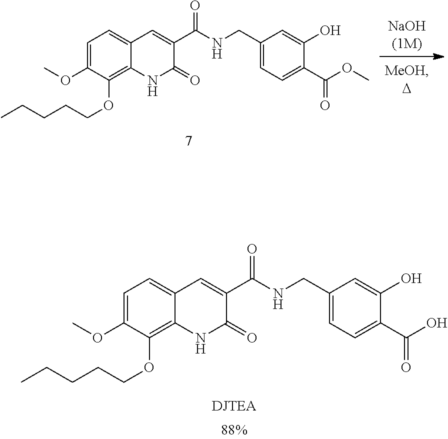

Prepared on 1.25 mmol scale using the same procedure as for DJTE3 (Section 5.4.2). Hydrolysis was followed by HPLC (SM=14.730 min, product=10.997 min) and was complete in 3 h. The reaction mixture was then cooled and the pH was adjusted to 4 with dil. aq. HCl, which caused the product to precipitate out of solution as a white solid which was collected and washed with water (20 ml), then ether (20 ml) and was dried in vacuo to give 0.499 g (88%) of a white powder. 1H-NMR (DMSO D6) 500 MHz: δ (ppm)=0.89 (3H, t, J=7.0 Hz, CH2CH2CH3), 1.30-1.45 (4H, m, CH2CH2CH3), 1.78 (2H, quin, J=7.2 Hz, CH2CH2CH2CH3), 3.93 (3H, s, COCH3), 4.00 (2H, t, J=6.9 Hz, OCH2CH2), 4.58 (2H, d, J=6.0 Hz, NHCH2C), 6.80-6.95 (2H, m, CCHCHCCHCOH), 7.14 (1H, d, J=8.9 Hz, CHCOCH3), 7.69 (1H, d, J=8.9 Hz, CHCHCOCH3), 7.75 (1H, d, J=8.5 Hz, CCHCHCCHCOH), 8.79 (1H, s, CCCHCCONH), 10.15 (1H, br.t, J=6.0 Hz, NHCH2C), 11.26 (1H, br.s, COH), 11.44 (1H, s, CNHCOC), 13.77 (1H, br.s, COOH). 13C-NMR (DMSO D6) 125 MHz: δ (ppm)=13.8 (CH3), 21.8 (CH2), 27.3 (CH2), 28.7 (CH2), 42.0 (NCH2), 56.3 (OCH3), 72.8 (OCH2), 109.2 (CH), 111.3 (C), 113.7 (C), 115.1 (CH), 117.8 (CH), 118.6 (C), 125.7 (CH), 130.3 (CH), 132.2 (C), 133.9 (C), 144.2 (CH), 147.7 (C), 154.7 (C), 161.1 (C), 162.1 (CO), 163.1 (CO), 171.6 (CO). IR Spectrum; solid state: v˜(cm-1)=3270, 3070, 2947, 1671, 1626, 1530, 1467, 1269, 1214. MS-ES (negative): 453.2 (M−H+). HPLC: 10.997 min, >97.2% purity.

Synthesis of DWIN1

### Synthesis of (2-Methyl-1H-indol-3-yl)-naphthalen-1-ylmethanone 15

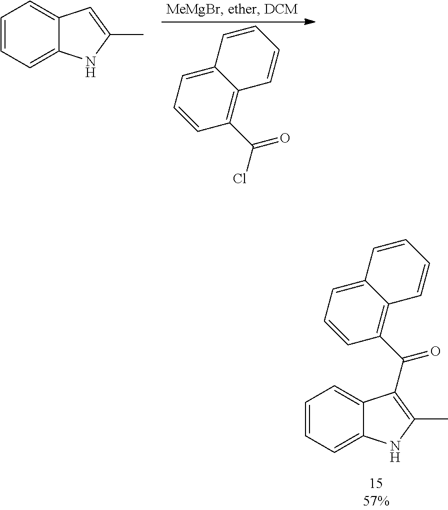

2-Methylindole (6.88 g, 52.46 mmol, 1 eq) was dissolved in ether (30 ml) and the solution was cooled to 0° C. MeMgBr (3M in ether, 62.95 ml, 62.95 mmol, 1.2 eq) was then added dropwise over 30 min and after the addition, the mixture was allowed to warm to ambient temperature. 1-Naphthoyl chloride (10 g, 52.46 mmol, 1 eq) in ether (15 ml) was added dropwise over 30 min and then the mixture was refluxed for 1 h, cooled and sat. aq. NH4Cl (200 ml) was added slowly to quench the reaction. The mixture was stirred until it was a pink slurry and the solids were then removed via filtration and were washed with water (50 ml). The solids were suspended in methanol (200 ml), a solution of NaOH (3 g) in water (100 ml) was added and the mixture was refluxed overnight. The solids were then filtered, washed with water (500 ml), washed with ether (250 ml) and were dried in vacuo. The solids were dissolved in DCM and were dry loaded onto silica and were then chromatographed in 1:1 CyH/EtOAc (Rf SM=0.9, Rf product=0.51, UV, KMnO4). This gave 10.847 g (70%) of the product as a pink solid. 1H-NMR (DMSO D6) 400 MHz: δ (ppm)=2.17 (3H, s, CH3), 3.34 (1H, s, NH), 6.94-6.99 (1H, m, NCCHCHCHCHC), 7.08-7.14 (1H, m, NCCHCHCHCHC), 7.25 (1H, br.d, J=8.0 Hz, NCCHCHCHCHC), 7.37 (1H, br.d, J=8.0 Hz, NCCHCHCHCHC), 7.44-7.58 (3H, m, CCHCHCHCCO, CCHCHCHCHCCCO), 7.61 (1H, dd, J=7.0 Hz, J=8.1 Hz, CCHCHCHCHCCCO), 7.83 (1H, br.d, J=8.3 Hz, CCHCHCHCCO), 8.03 (1H, br.d, J=8.2 Hz, CCHCHCHCHCCCO), 8.07 (1H, br.d, J=8.2 Hz, CCHCHCHCHCCCO). 13C-NMR (DMSO D6) 100 MHz: δ (ppm)=14.1 (CH3), 111.2 (CH), 113.7 (C), 120.1 (CH), 121.3 (CH), 122.0 (CH), 124.2 (CH), 124.7 (CH), 125.4 (CH), 126.2 (CH), 126.7 (CH), 126.9 (C), 128.2 (CH), 129.1 (CH), 129.3 (C), 133.1 (C), 134.9 (C), 140.5 (C), 145.7 (C), 191.9 (CO). IR Spectrum; evaporated film: v˜(cm-1)=3173, 1720, 1569, 1433, 1237, 1099, 1043. MS-ES (negative): 284.1 (M−H+). MS-ES (positive): 308.0 (M+Na+).

### Synthesis of 4-(2-Chloro-ethylamino)-2-methoxy-benzoic acid methyl ester 16

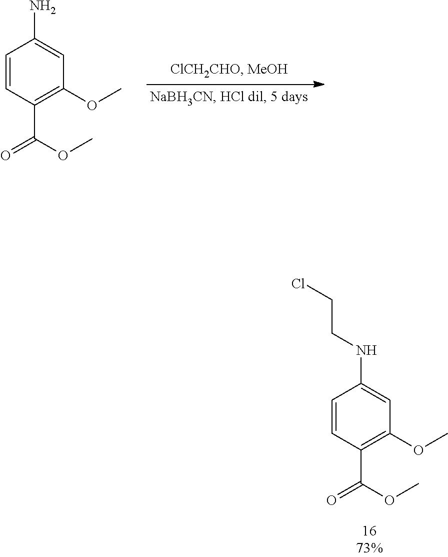

Methyl 4-amino-2-methoylbenzoate (2 g, 11.04 mmol, 1 eq) was dissolved in methanol (30 ml) and a 1:1 mixture (2 ml) of 6M aq. HCl and methanol was added. Chloroacetaldehyde (50% in water, 2.08 ml, 13.27 mmol, 1.2 eq) was added and the mixture was cooled to 0° C. NaBH3CN (0.78 g, 12.37 mmol, 1.12 eq) was added in portions over 2 min and the mixture was stirred for 5 days at ambient temperature. The mixture was poured into sat. aq. NaHCO3 (100 ml) and DCM (100 ml) was added, the pH was adjusted to 7-8 with dil. aq. HCl and the organic layer was poured off. The aqueous layer was then extracted with DCM (2×50 ml) and the combined organic layers were washed with water (2×100 ml), were washed with brine (50 ml), were dried over Na2SO4, filtered and the solvent was removed in vacuo. The product was purified via column chromatography eluted with a gradient from 1:1 to 1:3 CyH:EtOAc (Rf product=0.5, Rf SM=0.35 in 1:3 CyH:EtOAc, UV, KMnO4). This gave 1.968 g (73%) of white solid. 1H-NMR (CDCl3) 500 MHz: δ (ppm)=3.55 (2H, br.quart, J=5.1 Hz, ClCH2CH2), 3.71 (2H, t, J=5.9 Hz, ClCH2CH2), 3.82 (3H, s, COCH3), 3.86 (3H, s, COOCH3), 4.48 (1H, br.s, ClCH2CH2NH), 6.13 (1H, d, J=1.9 Hz, CCHCN), 6.19 (1H, dd, J=2.0 Hz, J=8.6 Hz, CCHCHCN), 7.77 (1H, d, J=8.6 Hz, CCHCHCN). 13C-NMR (CDCl3) 125 MHz: δ (ppm)=43.1 (CH2), 44.8 (CH2), 51.4 (CH3), 55.8 (CH3), 96.1 (CH), 104.1 (CH), 108.8 (C), 134.3 (CH), 152.2 (C), 161.8 (C), 166.1 (CO). IR Spectrum; evaporated film: v˜(cm-1)=3361, 2950, 2840, 1700, 1607, 1526, 1346, 1255, 1182, 1085. MS-ES (negative): 242.1 (M−H+), 244.1 (M−H+). MS-ES (positive): 244.1 (M+H+), 246.1 (M+H+).

### Synthesis of 2-Methoxy-4-{2-[2-methyl-3-(naphthalene-1-carbonyl)-indol-1-yl]-ethylamino}-benzoic acid methyl ester 17

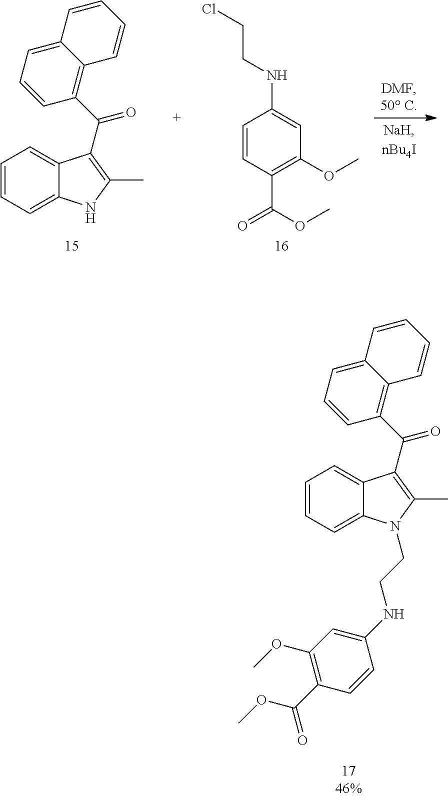

Indole 15 (2.303 g, 8.07 mmol, 1 eq) and nBu4NBr (50 mg) were dissolved in DMF (8 ml). Sodium hydride (60% dispersion in mineral oil, 0.339 g, 8.47 mmol, 1.05 eq) was added and the mixture was stirred for 15 min. Chloride 16 (1.967 g, 8.07 mmol, 1 eq) was dissolved in DMF (8 ml) and was then added rapidly to the reaction mixture and the reaction was heated to 50° C. overnight. After cooling, the reaction mixture was poured into water (100 ml) and DCM (100 ml) was added and the organic layer was poured off. The aqueous layer was then extracted with DCM (2×50 ml) and the combined organic layers were washed with water (2×100 ml), were washed with brine (50 ml), were dried over Na2SO4, filtered and the solvent was removed in vacuo. The product was purified via column chromatography eluted with a gradient from 1:1 to 1:1.3 CyH:EtOAc (Rf indole SM=0.5, Rf chloride 16=0.4, Rf product=0.2 in 1:1 CyH:EtOAc, UV, CAM). This gave 1.825 g (46%) of a foamy white solid. 1HNMR (CDCl3) 500 MHz: δ (ppm)=2.34 (3H, s, CCH3), 3.63 (3H, s, COCH3), 3.66 (2H, quart, J=5.8 Hz, NCH2CH2NHC), 3.82 (3H, s, COOCH3), 4.27 (1H, t, J=6.5 Hz, NCH2CH2NHC), 4.34 (2H, t, J=5.8 Hz, NCH2CH2NHC), 5.86 (1H, d, J=1.8 Hz, CCHCN), 6.10 (1H, dd, J=2.0 Hz, J=8.6 Hz, CCHCHCN), 7.04 (1H, t, J=7.6 Hz, NCCHCHCHCHC), 7.18 (1H, t, J=7.2 Hz, NCCHCHCHCHC), 7.25-7.30 (2H, m, NCCHCHCHCHC), 7.40-7.53 (4H, m, CCHCHCHCCO, CCHCHCHCHCCCO), 7.75 (1H, d, J=8.6 Hz, CCHCHCN), 7.91 (1H, br.d, J=8.2 Hz, CCHCHCHCCCO), 7.96 (1H, br.d, J=8.0 Hz, CCHCHCHCHCCCO), 8.08 (1H, br.d, J=8.4 Hz, CCHCHCHCHCCCO). 13C-NMR (CDCl3) 125 MHz: δ (ppm)=12.6 (CH3), 42.3 (CH2), 42.8 (CH2), 51.4 (CH3), 55.5 (CH3), 95.2 (CH), 103.8 (CH), 108.8 (C), 109.0 (CH), 115.5 (C), 121.6 (CH), 122.4 (CH), 122.6 (CH), 125.0 (CH), 125.4 (CH), 125.9 (CH), 126.3 (CH), 126.9 (CH), 127.2 (C), 128.3 (CH), 130.2 (CH), 130.3 (C), 133.8 (C), 134.3 (CH), 135.9 (C), 140.1 (C), 145.4 (C), 151.9 (C), 161.9 (C), 166.1 (CO), 193.5 (CO). IR Spectrum; evaporated film: v˜(cm-1)=3352, 3053, 2946, 1696, 1606, 1513, 1413, 1250, 1090. MS-ES (negative): 491.3 (M−H+). MS-ES (positive): 493.3 (M+H+).

### Synthesis of 2-Hydroxy-4-{2-[2-methyl-3-(naphthalene-1-carbonyl)-indol-1-yl]-ethylamino}-benzoic acid methyl ester 18

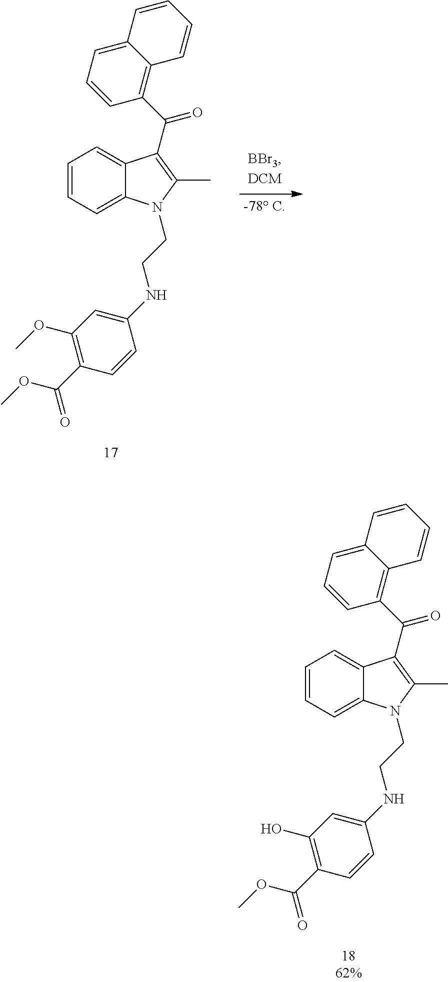

Methyl ether 17 (3.31 g, 6.72 mmol, 1 eq) was dissolved in DCM (50 ml) and the solution was cooled to −78° C. BBr3 (2.54 ml, 26.88 mmol, 4 eq) dissolved in DCM (50 ml) was then added dropwise over 2 min to the reaction and the reaction was stirred for 2 h at −78° C. The mixture was then warmed to ambient temperature and poured into sat. aq. NaHCO3 (100 ml) and the organic layer was poured off. The aqueous layer was then extracted with DCM (2×50 ml) and the combined organic layers were washed with water (2×100 ml), were washed with brine (50 ml), were dried over Na2SO4, filtered and the solvent was removed in vacuo. The product was purified via column chromatography eluted with 1:1 CyH:EtOAc (Rf product=0.78, Rf SM=0.33, UV, CAM). This gave 2.0 g (62%) of a foamy white solid. 1H-NMR (CDCl3) 500 MHz: δ (ppm)=2.31 (3H, s, CCH3), 3.59 (2H, quart, J=5.7 Hz, NCH2CH2NHC), 3.88 (3H, s, COOCH3), 4.30 (2H, t, J=5.9 Hz, NCH2CH2NHC), 4.40 (1H, t, J=6.4 Hz, NCH2CH2NHC), 5.93 (1H, dd, J=2.3 Hz, J=8.8 Hz, CCHCHCN), 6.04 (1H, d, J=2.2 Hz, CCHCN), 7.03 (1H, t, J=7.2 Hz, NCCHCHCHCHC), 7.17 (1H, t, J=7.2 Hz, NCCHCHCHCHC), 7.24 (1H, d, J=8.2 Hz, NCCHCHCHCHC), 7.26 (1H, d, J=7.5 Hz, NCCHCHCHCHC), 7.40-7.52 (4H, m, CCHCHCHCCO, CCHCHCHCHCCCO), 7.57 (1H, d, J=8.7 Hz, CCHCHCN), 7.91 (1H, d, J=8.2 Hz, CCHCHCHCCCO), 7.96 (1H, dd, J=2.5 Hz, J=6.8 Hz, CCHCHCHCHCCCO), 8.09 (1H, d, J=8.5 Hz, CCHCHCHCHCCCO), 11.03 (1H, s, COH). 13C-NMR (CDCl3) 125 MHz: δ (ppm)=12.5 (CH3), 42.0 (CH2), 42.1 (CH2), 51.6 (CH3), 97.5 (CH), 102.5 (C), 105.4 (CH), 109.1 (CH), 115.4 (C), 121.4 (CH), 122.3 (CH), 122.6 (CH), 125.0 (CH), 125.4 (CH), 125.7 (CH), 126.3 (CH), 126.9 (CH), 127.1 (C), 128.3 (CH), 130.1 (CH), 130.2 (C), 131.5 (CH), 133.7 (C), 135.9 (C), 140.1 (C), 145.7 (C), 153.0 (C), 163.8 (C), 170.4 (CO), 193.4 (CO). IR Spectrum; evaporated film: v˜(cm-1)=3399, 3335, 3054, 2950, 1656, 1624, 1516, 1439, 1412, 1348, 1270, 1197, 1159. MS-ES (negative): 477.3 (M−H+). MS-ES (positive): 479.2 (M+H+). HPLC: 15.012 min.

### Synthesis of 2-Hydroxy-4-{2-[2-methyl-3-(naphthalene-1-carbonyl)-indol-yl]-ethylamino}-benzoic acid DWIN1

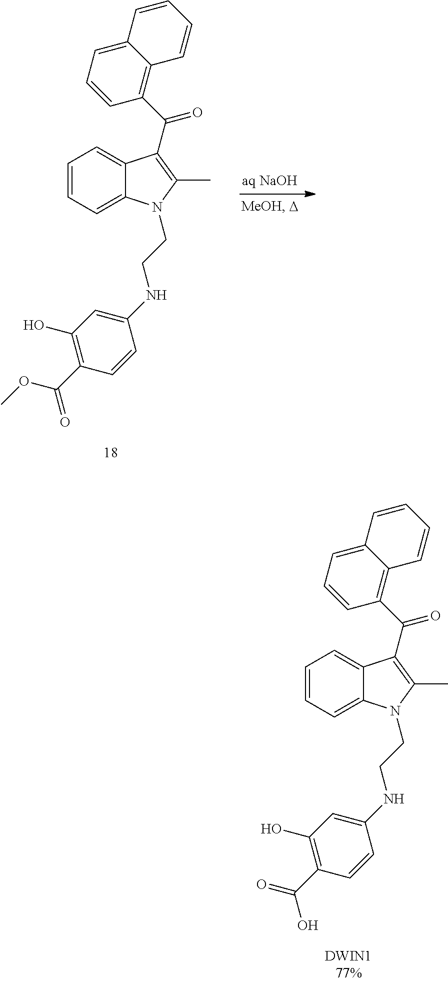

Methyl ester 18 (2 g, 4.18 mmol, 1 eq) and NaOH (0.67 g, 16.72 mmol, 4 eq) was stirred in methanol (50 ml) and water (17 ml), the mixture was heated to reflux temperature. Hydrolysis was followed by HPLC (SM=15.012 min, product=10.698 min) and once completed (overnight) the pH of the mixture was adjusted to 7 with dil. aq. HCl and the volatiles were removed in vacuo. The residues were azeotroped dry with MeOH and were then dry loaded onto silica and the product was purified via column chromatography eluted with a gradient from EtOAc to EtOAc/10% MeOH (Rf product=0.3, UV,CAM). This gave 1.5 g (77%) of a foamy yellow solid. 1H-NMR (DMSO D6) 500 MHz: δ (ppm)=2.08 (1H, s, COH). 2.21 (3H, s, CCH3), 3.48 (2H, quart, J=5.7 Hz, NCH2CH2NHC), 4.35 (2H, t, J=5.6 Hz, NCH2CH2NHC), 5.86 (1H, s, CCHCN), 5.92 (1H, d, J=8.7 Hz, CCHCHCN), 6.38 (1H, br.s, NCH2CH2NHC), 6.98 (1H, t, J=7.7 Hz, NCCHCHCHCHC), 7.11-7.20 (2H, m, NCCHCHCHCHC), 7.38-7.42 (2H, m, NCCHCHCHCHC, CCHCHCN), 7.46-7.51 (1H, m, CCHCHCHCCO), 7.51-7.58 (1H, m, CCHCHCHCCO, CCHCHCHCHCCCO), 7.87 (1H, d, J=8.5 Hz, CCHCHCHCCCO), 8.03 (1H, d, J=8.1 Hz, CCHCHCHCHCCCO), 8.07 (1H, d, J=8.1 Hz, CCHCHCHCHCCCO), 13.08 (1H, s, COOH). 13C-NMR (DMSO D6) 125 MHz: δ (ppm)=12.1 (CH3), 41.1 (CH2), 42.2 (CH2), 96.5 (CH), 102.8 (C), 103.5 (CH), 110.1 (CH), 113.9 (C), 120.1 (CH), 121.6 (CH), 122.0 (CH), 124.8 (CH), 124.9 (CH), 125.2 (CH), 126.2 (CH), 126.5 (C), 126.8 (CH), 128.1 (CH), 129.4 (C), 129.4 (CH), 131.0 (CH), 133.1 (C), 135.8 (C), 140.2 (C), 146.3 (C), 153.1 (C), 163.8 (C), 172.5 (CO), 191.9 (CO). IR Spectrum; evaporated film: v˜(cm-1)=3361, 1923, 1701, 1576, 1498, 1348, 1227, 1085. MS-ES (negative): 463.2 (M−H+). MS-ES (positive): 465.2 (M+H+). HPLC: 10.698 min, 97.0% purity.

### Synthesis of DWIN2

### 5-Amino-2-hydroxy-benzoic acid methyl ester 19

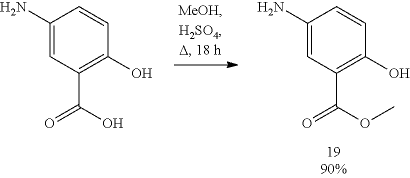

5-Methyl salicylic acid (10 g, 65.3 mmol, 1 eq) was dissolved in methanol (80 ml) and conc. H2SO4 (10 ml) was added carefully. The mixture was heated to reflux temperature overnight and was then allowed to cool to ambient temperature and was then poured into a separating funnel and water (100 ml) and DCM (100 ml) were added. The pH was adjusted to 7 with dil. aq. NaOH and the organic layer was poured off. The aqueous layer was then extracted with DCM (2×50 ml) and the combined organic layers were washed with water (2×100 ml), were washed with brine (50 ml), were dried over Na2SO4, filtered and the solvent was removed in vacuo. This gave 9.778 g (62%) of an off white solid. 1H-NMR (DMSO D6) 500 MHz: δ (ppm)=3.85 (3H, s, CH3), 4.78 (2H, br.s, NH2), 6.70 (1H, d, J=8.7 Hz, CCHCHCN), 6.82 (1H, dd, J=2.9 Hz, J=8.7 Hz, CCHCHCN), 7.01 (1H, d, J=2.9 Hz, CCHCN), 9.74 (1H, s, COH). 13CNMR (DMSO D6) 125 MHz: δ (ppm)=52.1 (CH3), 112.1 (C), 112.8 (CH), 117.5 (CH), 123.0 (CH), 141.0 (C), 151.5 (C), 169.6 (CO). IR Spectrum; evaporated film: v˜(cm-1)=3408, 3328, 3220, 3082, 2958, 1675, 1616, 1485, 1441, 1303, 1231, 1083. MS-ES (positive): 168.06 (M+H+).

### 5-Amino-2-methoxy-benzoic acid methyl ester 20

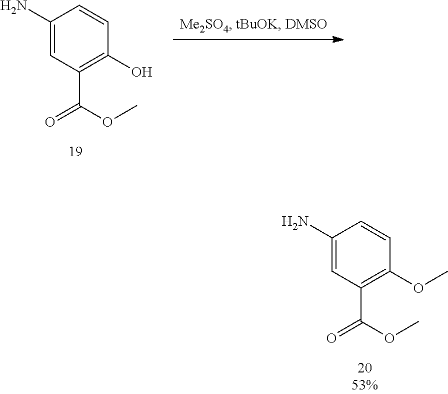

Phenol 19 (5 g, 29.9 mmol, 1 eq) and tBuOK (3.35 g, 29.9 mmol, 1 eq) were stirred in DMSO (70 ml) for 2 h at ambient temperature. Dimethylsulphate (3 ml, 3.17 mmol, 1.06 eq) was added and the mixture was stirred for 5 min before being poured into water (100 ml) and EtOAc (100 ml). The pH was adjusted to 7 with dil. aq. HCl and the organic layer was poured off. The aqueous layer was then extracted with EtOAc (2×50 ml) and the combined organic layers were washed with water (2×100 ml), were ashed with brine (50 ml), were dried over Na2SO4, filtered and the solvent was removed in vacuo. The product was purified via column chromatography eluted with 1:1 EtOAc:CyH (Rf SM=0.4, Rf product=0.2, UV,CAM). This gave 3.152 g (53%) of a brown oil. 1H-NMR (CDCl3) 500 MHz: δ (ppm)=3.50 (2H, br.s, NH2), 3.83 (3H, s, COCH3), 3.87 (3H, s, COOCH3), 6.80-6.85 (2H, m, CCHCHCN), 7.15 (1H, br.s, CCHCN). 13C-NMR (CDCl3) 125 MHz: δ (ppm)=51.9 (CH3), 56.8 (CH3), 114.2 (CH), 117.9 (CH), 120.2 (CH), 120.6 (C), 139.6 (C), 152.3 (C), 166.7 (CO). IR Spectrum; evaporated film: v˜(cm-1)=3432, 3360, 3230, 2951, 2837, 1717, 1627, 1501, 1441, 1313, 1227, 1081, 1023. MS-ES (positive): 182.07 (M+H+).

### 5-(2-Chloro-ethylamino)-2-methoxy-benzoic acid methyl ester 21

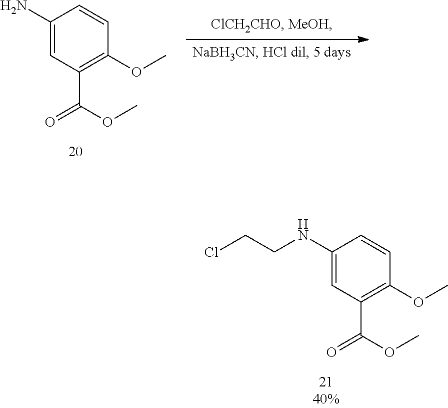

Prepared on 3.53 mmol scale using the same procedure as for 16 (Section 5.6.2). The product was purified via column chromatography eluted with a gradient from 1:1 to 1:3 CyH:EtOAc (Rf product=0.77, Rf SM=0.4 in 1:3 CyH:EtOAc, UV, KMnO4). This gave 0.348 g (40%) of a white solid. 1HNMR (CDCl3) 500 MHz: δ (ppm)=3.47 (2H, t, J=5.8 Hz, ClCH2CH2), 3.70 (2H, t, J=5.9 Hz, ClCH2CH2), 3.83 (3H, s, COCH3), 3.88 (3H, s, COOCH3), 3.80-4.00 (1H, br.s, ClCH2CH2NH), 6.68 (1H, dd, J=3.0 Hz, J=8.8 Hz, CCHCHCN), 6.87 (1H, d, J=8.9 Hz, CCHCHCN), 7.11 (1H, d, J=3.0 Hz, CCHCN). 13C-NMR (CDCl3) 125 MHz: δ (ppm)=43.5 (CH2), 46.3 (CH2), 52.0 (CH3), 56.9 (CH3), 114.4 (CH), 116.2 (CH), 118.9 (CH), 120.9 (C), 140.6 (C), 152.3 (C), 166.8 (CO). IR Spectrum; evaporated film: v˜(cm-1)=3381, 1951, 2838, 1720, 1617, 1584, 1505, 1437, 1235, 1081. MS-ES (positive): 244.1 (M+H+), 246.1 (M+H+).

### 2-Methoxy-5-{2-[2-methyl-3-(naphthalene-1-carbonyl)-indol-1-yl]-ethylamino}-benzoic acid methyl ester 22

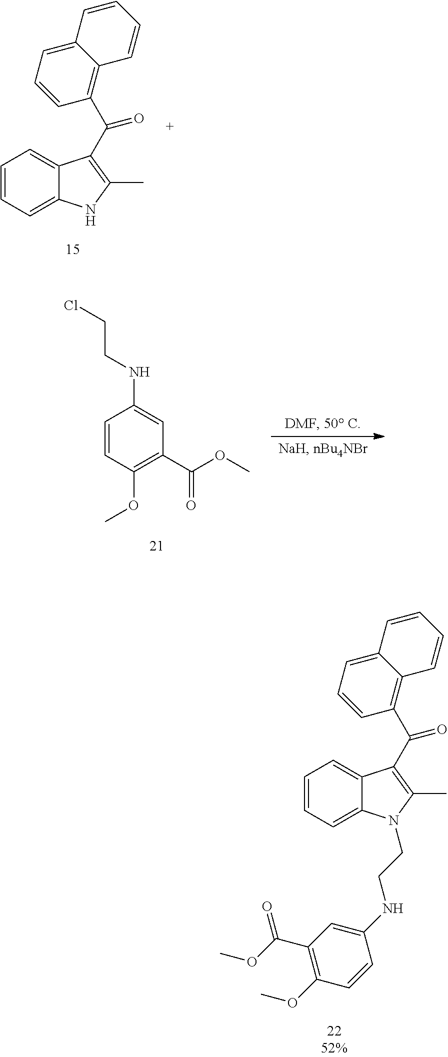

Prepared on 0.82 mmol scale using the same procedure as for 17 (Section 5.6.3). The product was purified via column chromatography eluted with a gradient from 1:1 to 1:1.3 CyH:EtOAc (Rf indole SM=0.5, Rf Cl SM=0.4, Rf product=0.36 in 1:1 CyH:EtOAc, UV, CAM). This gave 0.209 g (52%) of a foamy white solid. 1H-NMR (CDCl3) 500 MHz: 6 (ppm)=2.37 (3H, s, CCH3), 3.56 (2H, t, J=6.0 Hz, NCH2CH2NHC), 3.65 (1H, br.s, NCH2CH2NHC), 3.83 (3H, s, COCH3), 3.86 (3H, s, COOCH3), 4.32 (2H, t, J=5.8 Hz, NCH2CH2NHC), 6.63 (1H, dd, J=3.0 Hz, J=8.9 Hz, CCHCHCN), 6.83 (1H, d, J=8.9 Hz, CCHCHCN), 7.02 (1H, d, J=3.1 Hz, CCHCN), 7.03 (1H, t, J=8.0 Hz, NCCHCHCHCHC), 7.18 (1H, t, J=8.1 Hz, NCCHCHCHCHC), 7.26 (1H, br.d, J=8.0 Hz, NCCHCHCHCHC), 7.29 (1H, br.d, J=8.2 Hz, NCCHCHCHCHC), 7.41-7.45 (1H, m, CCHCHCHCCO), 7.46-7.54 (3H, m, CCHCHCHCCO, CCHCHCHCHCCCO), 7.91 (1H, br.d, J=8.2 Hz, CCHCHCHCCCO), 7.96 (1H, br.d, J=7.9 Hz, CCHCHCHCHCCCO), 8.10 (1H, br.d, J=8.4 Hz, CCHCHCHCHCCCO). 13C-NMR (CDCl3) 125 MHz: 6 (ppm)=12.6 (CH3), 42.6 (CH2), 43.4 (CH2), 52.0 (CH3), 56.9 (CH3), 109.2 (CH), 114.5 (CH), 115.0 (CH), 115.3 (C), 118.2 (CH), 120.8 (C), 121.4 (CH), 122.2 (CH), 122.5 (CH), 125.0 (CH), 125.5 (CH), 125.7 (CH), 126.2 (CH), 126.9 (CH), 127.2 (C), 128.2 (CH), 130.0 (CH), 130.3 (C), 133.8 (C), 136.0 (C), 140.2 (C), 140.4 (C), 145.7 (C), 152.7 (C), 166.7 (CO), 193.3 (CO). IR Spectrum; evaporated film: v˜(cm-1)=3378, 3051, 2998, 2838, 1719, 1609, 1507, 1412, 1234, 1085. MS-ES (negative): 491.3 (M−H+). MS-ES (positive): 493.3 (M+H+).

### 2-Hydroxy-5-{2-[2-methyl-3-(naphthalene-1-carbonyl)-indol-1-yl]-ethylamino}-benzoic acid methyl ester 23

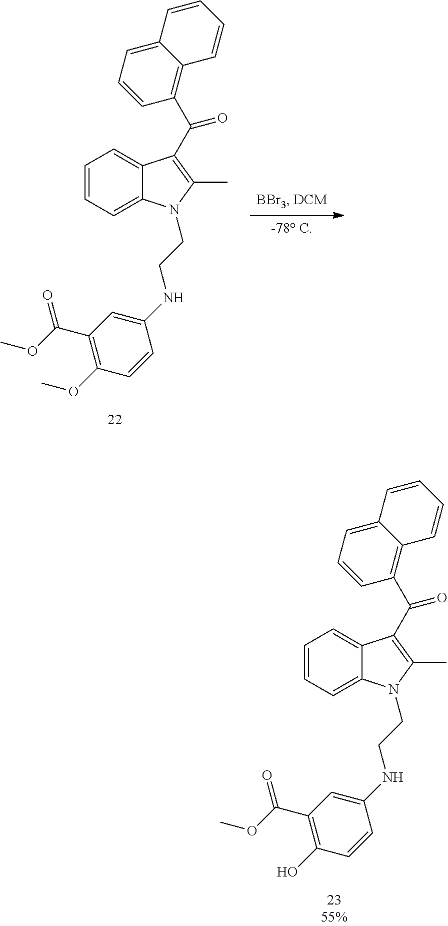

Prepared on 2.03 mmol scale using the same procedure as for 16 (Section 5.6.4). The product was purified via column chromatography eluted with 1:1 CyH:EtOAc (Rf product=0.45, Rf SM=0.33 in 4:6 CyH:EtOAc, UV, CAM). This gave 0.534 g (55%) of a foamy off white solid. 1H-NMR (CDCl3) 500 MHz: δ (ppm)=1.55 (1H, br.s, NCH2CH2NHC), 2.42 (3H, s, CCH3), 3.58 (2H, t, J=6.2 Hz, NCH2CH2NHC), 3.89 (3H, s, COOCH3), 4.38 (2H, t, J=6.1 Hz, NCH2CH2NHC), 6.76 (1H, dd, J=2.7 Hz, J=8.9 Hz, CCHCHCN), 6.85 (1H, d, J=8.9 Hz, CCHCHCN), 6.69 (1H, d, J=2.6 Hz, CCHCN), 7.03 (1H, t, J=7.3 Hz, NCCHCHCHCHC), 7.19 (1H, t, J=7.2 Hz, NCCHCHCHCHC), 7.22 (1H, d, J=8.0 Hz, NCCHCHCHCHC), 7.32 (1H, d, J=8.2 Hz, NCCHCHCHCHC), 7.44 (1H, t, J=8.2 Hz, CCHCHCHCCO), 7.47-7.53 (3H, m, CCHCHCHCCO, CCHCHCHCHCCCO), 7.91 (1H, br.d, J=8.2 Hz, CCHCHCHCCCO), 7.97 (1H, br.d, J=7.5 Hz, CCHCHCHCHCCCO), 8.10 (1H, br.d, J=8.4 Hz, CCHCHCHCHCCCO), 10.22 (1H, s, COH). 13C-NMR (CDCl3) 125 MHz: δ (ppm)=12.6 (CH3), 42.6 (CH2), 43.8 (CH2), 52.3 (CH3), 109.2 (CH), 111.4 (CH), 112.3 (C), 115.4 (C), 118.7 (CH), 121.5 (CH), 122.2 (CH), 122.5 (CH), 123.4 (CH), 125.0 (CH), 125.5 (CH), 125.8 (CH), 126.3 (CH), 126.9 (CH), 127.2 (C), 128.3 (CH), 130.1 (CH), 130.3 (C), 133.8 (C), 136.1 (C), 138.8 (C), 140.2 (C), 145.6 (C), 155.0 (C), 170.2 (CO), 193.4 (CO). IR Spectrum; evaporated film: v˜(cm-1)=3584, 3348, 3053, 2951, 1678, 1613, 1503, 1440, 1411, 1290, 1207, 1088. MS ES (negative): 477.3 (M−H+). MS-ES (positive): 479.2 (M+H+). HPLC: 14.462 min.

### 2-Hydroxy-5-{2-[2-methyl-3-(naphthalene-1-carbonyl)-indol-1-yl]-ethylamino}-benzoic acid DWIN2

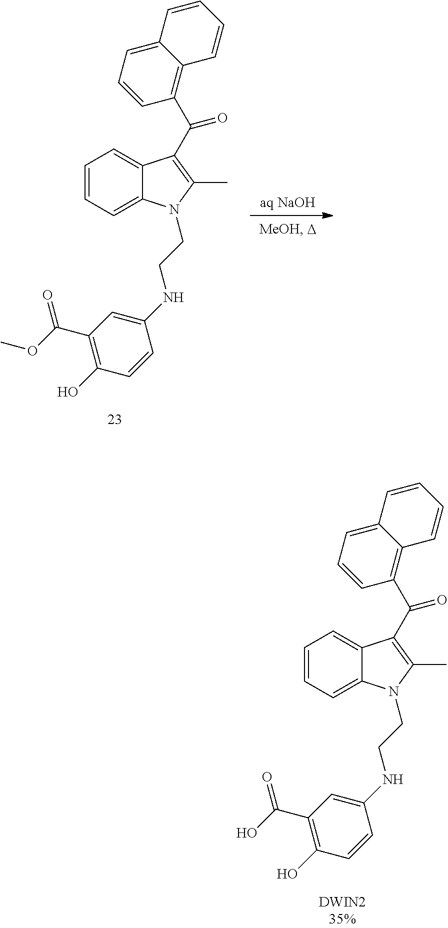

Prepared on 1.78 mmol scale using the same procedure as for DWIN1 (Section 5.6.4). Hydrolysis was followed by HPLC (SM=14.462 min, product=10.120 min) and was completed in 1 h. The product was purified via column chromatography eluted with a gradient from EtOAc to EtOAc/10% MeOH (Rf product=0.25, UV,CAM). This gave 290 mg (35%) of a foamy yellow solid. 1H-NMR (CDCl3) 500 MHz: δ (ppm)=1.91 (1H, s, COH), 2.28 (3H, s, CCH3), 3.35 (2H, t, J=5.9 Hz, NCH2CH2NHC), 4.33 (2H, t, J=6.2 Hz, NCH2CH2NHC), 5.08 (1H, br.s, NCH2CH2NHC), 6.48 (1H, d, J=8.6 Hz, CCHCHCN), 6.52 (1H, dd, J=2.9 Hz, J=8.6 Hz, CCHCHCN), 6.96 (1H, t, J=7.3 Hz, NCCHCHCHCHC), 7.06 (1H, d, J=5.7 Hz, NCCHCHCHCHC), 7.07 (1H, s, CCHCN), 7.15 (1H, t, J=8.2 Hz, NCCHCHCHCHC), 7.46-7.50 (2H, m, CCHCHCHCHCCCO), 7.53-7.64 (3H, m, CCHCHCHCCO, NCCHCHCHCHC), 7.87 (1H, d, J=8.4 Hz, CCHCHCHCCCO), 8.03 (1H, d, J=8.2 Hz, CCHCHCHCHCCCO), 8.08 (1H, d, J=8.2 Hz, CCHCHCHCHCCCO), 13.36 (3H, br.s, COOCH). 13C-NMR (CDCl3) 125 MHz: δ (ppm)=12.2 (CH3), 42.3 (CH2), 43.0 (CH2), 110.2 (CH), 113.1 (CH), 113.7 (C), 115.9 (CH), 117.6 (CH), 119.6 (C), 120.0 (CH), 121.6 (CH), 122.0 (CH), 124.8 (2×CH), 125.3 (CH), 126.2 (CH), 126.4 (C), 126.8 (CH), 128.2 (CH), 129.4 (CH, C), 133.1 (C), 135.9 (C), 138.8 (C), 140.3 (C), 146.4 (C), 153.7 (C), 172.4 (CO), 191.9 (CO). IR Spectrum; evaporated film: v˜(cm-1)=3407, 3045, 2919, 1701, 1565, 1486, 1408, 1353, 1227, 1085. MS-ES (negative): 463.3 (M−H+). MS-ES (positive): 465.3 (M+H+). HPLC: 10.120 min, 96.3% purity.

### Synthesis of DWIN8

### (2-Methyl-1H-indol-3-yl)-acetic acid ethyl ester 30

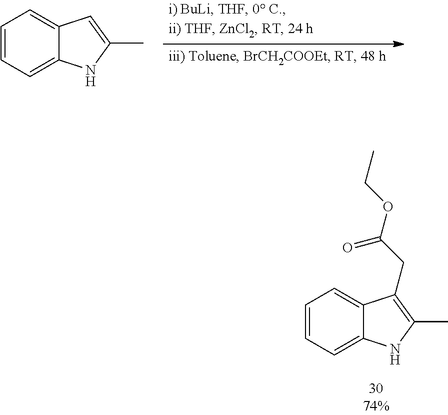

2-Methylindole (15.1 g, 0.115 mol, 1 eq) was dried under high vacuum and then dissolved in dry THF (100 ml) and cooled to 0° C. nButyllithium (1.6 M in hexanes, 77 ml, 0.115 mol, 1 eq) was added at a rate of 80 ml/h via a syringe pump. Reaction mixture was stirred at 0° C. for 15 min then a solution of anhydrous ZnCl2 (15.7 g, 0.115 mol, 1 eq) in THF (100 ml) was added to the reaction mixture. Reaction mixture was stirred at ambient temperature for 20 h then the THF was removed in vacuo. The residue was redissolved in dry toluene (50 ml) and bromoacetic acid ethyl ester (19 ml, 0.172 mol, 1.5 eq) was added and the reaction was stirred for 2 days. The mixture was then poured into water (200 ml) and was extracted with EtOAc (3×100 ml), the combined organic layers were then washed with water (100 ml), sat. aq. NaHCO3 (100 ml), brine (50 ml), were dried over Na2SO4, filtered and the solvent was removed in vacuo. The residues were then dry-flash chromatographed through a silica plug eluted with a gradient from toluene to 1:1 toluene:DCM to DCM to elute the product (Rf prod=0.27 in 1:1 CyH:EtOAc, UV, CAM). This gave 18.5 g (74%) of a yellowbrown oil. 1H-NMR (CDCl3) 500 MHz: δ (ppm)=1.26 (3H, t, J=7.1 Hz, CH2CH3), 2.41 (3H, s, CH3), 3.71 (2H, s, CCH2CO), 4.16 (2H, quart, J=7.1 Hz, CH2CH3), 7.10-7.17 (1H, m, NHCCHCHCHCHC), 7.27 (1H, d, J=6.0 Hz, NHCCHCHCHCHC), 7.57 (1H, d, J=7.0 Hz, NHCCHCHCHCHC), 7.89 (1H, br.s, NH). 13C-NMR (CDCl3) 125 MHz: δ (ppm)=11.6 (CH3), 14.2 (CH3), 30.5 (CH2), 60.6 (CH2), 104.7 (C), 110.2 (CH), 118.1 (CH), 119.5 (CH), 121.2 (CH), 128.5 (C), 132.6 (C), 135.1 (C), 172.0 (CO). IR Spectrum; evaporated film: v˜(cm-1)=3393, 3053, 2980, 2927, 1724, 1463, 1304, 1172, 1031. MS-ES (negative): 216.1 (M−H+). MS-ES (positive): 218.2 (M+H+).

### [1-(2,3-Dichloro-benzoyl)-2-methyl-1H-indol-3-yl]-acetic acid ethyl ester 31

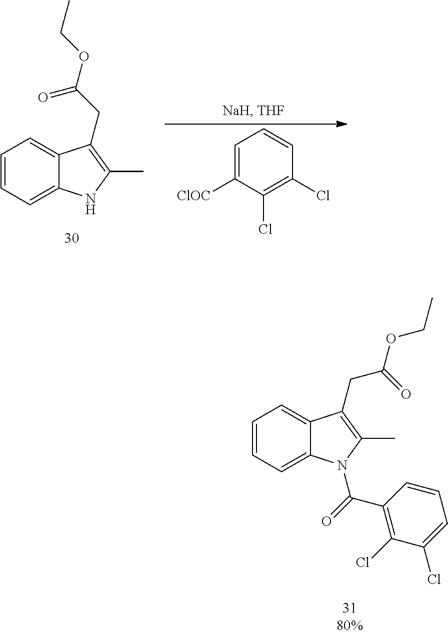

Indole 30 (5 g, 23.0 mmol, 1 eq) was dissolved in DMF (50 ml) and was cooled to 0° C. Sodium hydride (60% dispersion in mineral oil, 1.01 g, 25.31 mmol, 1.1 eq) was added and the mixture was stirred for 30 min. 2, 3-dichlorobenzoyl chloride (5.06 g, 24.16 mmol, 1.05 eq) was dissolved in DMF (25 ml) and this solution was added to the reaction over 2 min and the mixture was stirred overnight at ambient temperature. The mixture was poured into water (100 ml) and DCM (100 ml) and the organic layer was poured off. The aqueous layer was then extracted with DCM (2×50 ml) and the combined organic layers were washed with water (2×100 ml), were washed with brine (50 ml), were dried over Na2SO4, filtered and the solvent was removed in vacuo. The product was purified via column chromatography eluted with a gradient from 4:1 to 1:1 CyH:EtOAc (Rf product=0.4, Rf SM=0.27, UV, CAM). This gave 7.224 g (80%) of a yellow-green oil. 1H-NMR (CDCl3) 500 MHz: δ (ppm)=1.24 (3H, t, J=7.1 Hz, CH2CH3), 2.26 (3H, s, CH3), 3.66 (2H, s, CCH2CO), 4.14 (2H, quart, J=7.1 Hz, CH2CH3), 7.13 (1H, t, J=7.3 Hz NCCHCHCHCHC), 7.24 (1H, t, J=7.6 Hz NCCHCHCHCHC), 7.32 (1H, d, J=8.3 Hz, NCCHCHCHCHC), 7.37 (1H, dd, J=7.6 Hz, J=7.6 Hz, CCHCHCHCCl), 7.39 (1H, dd, J=2.0 Hz, J=7.6 Hz, CCHCHCHCCl), 7.50 (1H, d, J=7.8 Hz NCCHCHCHCHC), 7.60 (1H, dd, J=2.0 Hz, J=7.6 Hz, CCHCHCHCCl). 13C-NMR (CDCl3) 125 MHz: δ (ppm)=13.5 (CH3), 14.2 (CH3), 30.3 (CH2), 61.0 (CH2), 114.3 (C), 114.6 (CH), 118.4 (CH), 123.8 (CH), 124.3 (CH), 127.2 (CH), 128.1 (CH), 130.2 (C), 130.3 (C), 132.5 (CH), 134.3 (C), 134.4 (C), 135.8 (C), 138.4 (C), 165.8 (CO), 170.6 (CO). IR Spectrum; evaporated film: v˜(cm-1)=3068, 2980, 2931, 1734, 1687, 1456, 1358, 1320, 1160. MS-ES (positive): 390.1 (M+H+), 392.1 (M+H+).

### [1-(2,3-Dichloro-benzoyl)-2-methyl-1H-indol-3-yl]-acetaldehyde 32

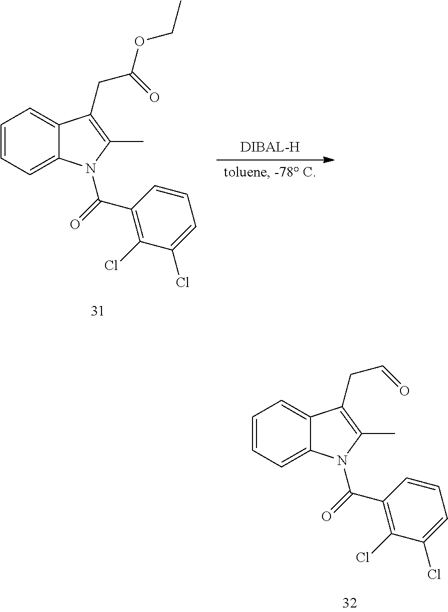

Ester 31 (3.784 g, 9.70 mmol, 1 eq) was dissolved in toluene (20 ml) and was cooled to −78° C. DIBAL-H (1.5M in toluene, 9.70 ml, 14.54 mmol, 1.5 eq) was added at a rate of 3 ml/min via a syringe pump, after the addition was complete the mixture was stirred for a further 30 min. Methanol (10 ml) was added at −78° C. at 6 ml/min via a syringe pump, and then as the mixture warmed to ambient temperature, dil. aq. HCl (2M, 50 ml) was added. Once the solution had cleared, the organic layer was poured off. The aqueous layer was then extracted with EtOAc (2×50 ml) and the combined organic layers were washed with water (2×100 ml), were washed with brine (50 ml), were dried over Na2SO4, filtered and the solvent was removed in vacuo. The product was not isolated and was used directly in the next step.

### 4-{2-[1-(2,3-Dichloro-benzoyl)-2-methyl-1H-indol-3-yl]-ethylamino}-2-hydroxy-benzoic acid benzyl ester

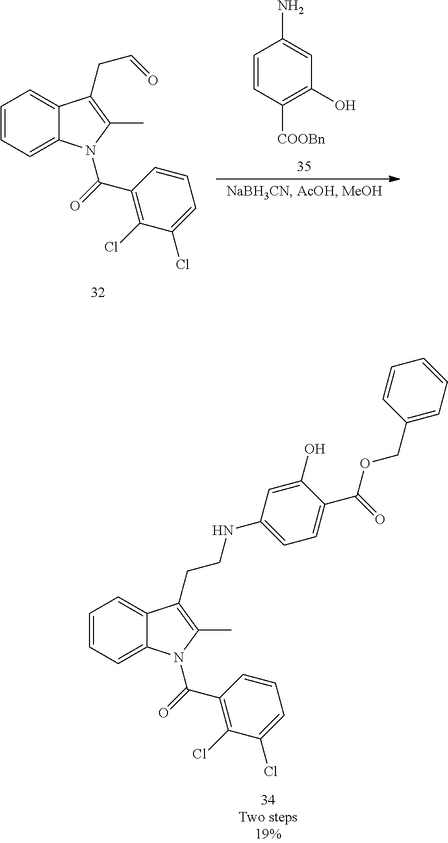

Aldehyde 32 (ca. 3.36 g, 9.70 mmol, 1 eq) and amine 35 (was dissolved in methanol (20 ml), glacial acetic acid (2.1 ml) was added and the mixture was cooled to 0° C. NaBH3CN (1.34 g, 21.33 mmol, 2.2 eq) was added in portions and the mixture was stirred overnight at ambient temperature. The mixture was poured into sat. aq. NaHCO3 (100 ml) and DCM (100 ml) was added, the pH was adjusted to 7-8 with dil. aq. NaOH and the organic layer was poured off. The aqueous layer was then extracted with DCM (2×50 ml) and the combined organic layers were washed with water (2×100 ml), were washed with brine (50 ml), were dried over Na2SO4, filtered and the solvent was removed in vacuo. The product was purified via column chromatography eluted with a gradient from 4:1 to 1:1 CyH:diethylether (Rf 31=0.5, Rf product=0.35, Rf 33 & 35=0.3 in 1:1 CyH:diethylether, UV, CAM) and was rechromatographed eluted with a gradient from toluene to toluene/3% diethylether (Rf 31=0.7, Rf product=0.63, Rf 33 & 35=0.5 in 9:1 toluene:diethylether, UV, CAM). This gave 1.037 g (19%) of a foamy yellow solid. 1H-NMR (CDCl3) 500 MHz: δ (ppm)=2.10 (3H, s, CH3), 2.97 (2H, t, J=6.6 Hz, CH2CH2NH), 3.46 (2H, br.t, J=6.3 Hz, CH2CH2NH), 4.21 (1H, br.s, CH2CH2NH), 5.32 (2H, s, CH2Ph), 6.01 (1H, dd, J=2.3 Hz, J=8.8 Hz, CCHCHCN), 6.09 (1H, d, J=2.3 Hz, CCHCN), 7.15-7.20 (1H, m, NCCHCHCHCHC), 7.23-7.27 (1H, m, NCCHCHCHCHC), 7.34-7.47 (9H, m, NCCHCHCHCHC, CCHCHCHCCl, Ph), 7.63 (1H, d, J=8.9 Hz, CCHCHCN), 7.64 (1H, dd, J=2.3 Hz, J=6.9 Hz, CCHCHCHCCl), 10.97 (1H, s, COH). 13CNMR (CDCl3) 125 MHz: δ (ppm)=13.5 (CH3), 23.7 (CH2), 42.4 (CH2), 66.1 (CH2), 97.7 (CH), 101.9 (C), 105.6 (CH), 114.9 (CH), 117.9 (CH & C), 123.9 (CH), 124.4 (CH), 127.3 (CH), 128.1 (CH), 128.2 (CH), 128.2 (CH), 128.6 (CH), 130.2 (C), 130.2 (C), 131.4 (CH), 132.6 (CH), 133.5 (C), 134.3 (C), 136.0 (C×2), 138.4 (C), 153.9 (C), 164.0 (C), 165.8 (CO), 169.8 (CO). IR Spectrum; evaporated film: v˜(cm-1)=3408, 3071, 2930, 1651, 1527, 1455, 1378, 1268, 1155. MS-ES (negative): 571.2 (M−H+), 573.1 (M−H+). MS-ES (positive): 573.2 (M+H+), 575.1 (M+H+).

### 4-Amino-2-hydroxy-benzoic acid benzyl ester 35

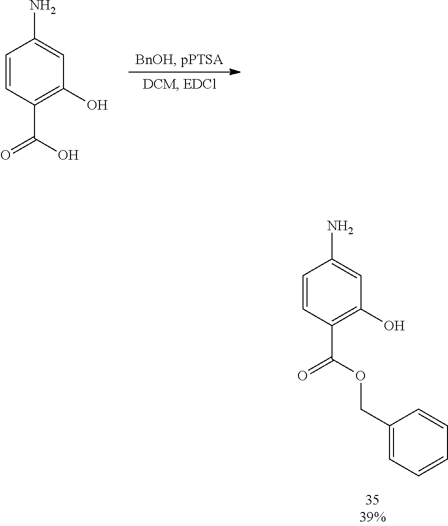

4-Aminosalicylic acid (3 g, 19.6 mmol, 1 eq), pyridinium ptoluenesulphonic acid (0.5 g, 1.96 mmol, 0.1 eq) and N-(3-dimethylaminopropyl)-N′-ethylcarbodiimide.HCl (6.57 g, 34.3 mmol, 1.75 eq) were dissolved in DCM (15 ml) and benzyl alcohol (3.05 ml, 29.4 mmol, 1.5 eq) was added. The reaction was stirred overnight and then was poured into water (50 ml) and DCM (50 ml). The pH was adjusted to 7 with dil. aq. NaOH and the organic layer was poured off. The aqueous layer was then extracted with DCM (2×50 ml) and the combined organic layers were washed with water (2×100 ml), were washed with brine (50 ml), were dried over Na2SO4, filtered and the solvent was removed in vacuo. The product was purified via column chromatography eluted with 2:3 EtOAc:CyH (Rf product=0.61, Rf BnOH=0.52, UV, CAM). Benzyl alcohol that coeluted with the product was later removed via trituration with cyclohexane (10 ml), the product was filtered off as a white powder (1.851 g, 39%). 1H-NMR (CDCl3) 500 MHz: δ (ppm)=4.09 (2H, br.s, NH2), 5.33 (2H, s, CH2), 6.13 (1H, dd, J=2.2 Hz, J=8.6 Hz, CCHCHCN), 6.16 (1H, d, J=2.2 Hz, CCHCHCN), 7.32-7.45 (5H, m, Ph), 7.67 (1H, d, J=8.6 Hz CCHCN), 10.92 (1H, s, COH). 13C-NMR (CDCl3) 125 MHz: δ (ppm)=66.2 (CH2), 100.7 (CH), 103.0 (C), 106.8 (CH), 128.1 (CH Ph), 128.2 (CH Ph), 128.6 (CH Ph), 131.7 (CH), 135.9 (C), 153.4 (C), 163.7 (C), 169.8 (CO). IR Spectrum; evaporated film: v˜(cm-1)=3460, 3370, 1637, 1511, 1385, 1275, 1152. MS-ES (positive): 242.1 (M+H+), 244.2 (M+H+). HPLC: 21.067 min.

### 4-{2-[1-(2,3-Dichloro-benzoyl)-2-methyl-1H-indol-3-yl]-ethylamino}-2-hydroxy-benzoic acid DWIN8

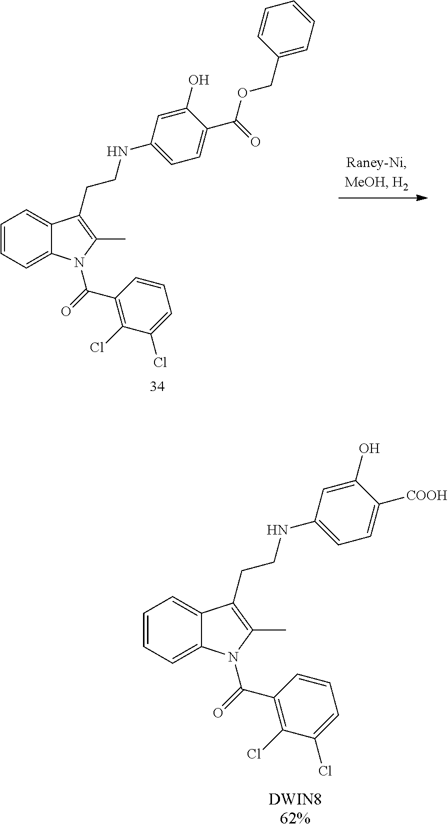

Benzylester 34 (1 g, 1.74 mmol, 1 eq) was dissolved in methanol (160 ml) and Raney-Ni (slurry in water, ˜200 mg washed twice with MeOH) was added. The mixture was purged with nitrogen and then with hydrogen and then was left stirring for 2 h with a hydrogen balloon attached. Hydrogenolysis was followed by HPLC (SM=21.098 min, product=14.405 min). The reaction was purged with nitrogen and then filtered through a celite plug, the plug was washed with MeOH (100 ml) and the solvent was removed in vacuo to give 0.52 g (62%) of a foamy yellow solid. 1H-NMR (DMSO D6) 500 MHz: δ (ppm)=1.97 (3H, s, CH3), 2.87 (2H, t, J=6.9 Hz, CH2CH2NH), 3.29 (2H, t, J=6.8 Hz, CH2CH2NH), 5.90 (1H, d, J=2.1 Hz, CCHCN), 6.04 (1H, dd, J=2.1 Hz, J=8.8 Hz, CCHCHCN), 6.44 (1H, J=6.6 Hz, CH2CH2NH), 7.19 (1H, t, J=7.2 Hz, NCCHCHCHCHC), 7.27 (1H, t, J=7.2 Hz, NCCHCHCHCHC), 7.41 (1H, d, J=8.7 Hz, CCHCHCN), 7.43 (1H, br.d, J=8.2 Hz, NCCHCHCHCHC), 7.56 (1H, dd, J=7.8 Hz, J=7.8 Hz, CCHCHCHCCl), 7.57 (1H, br.d, J=7.6 Hz, NCCHCHCHCHC), 7.61 (1H, dd, J=1.6 Hz, J=7.6 Hz, CCHCHCHCCl), 7.90 (1H, dd, J=1.6 Hz, J=8.0 Hz, CCHCHCHCCl), 12.05 (1H, br.s, COH), acid signal not obvious. 13C-NMR (DMSO D6) 125 MHz: δ (ppm)=12.9 (CH3), 23.1 (CH2), 41.6 (CH2), 96.3 (CH), 102.0 (C), 104.4 (CH), 114.4 (CH), 118.2 (CH), 118.3 (C), 123.7 (CH), 124.0 (CH), 127.8 (CH), 128.2 (C), 129.3 (CH), 130.1 (C), 130.9 (CH), 132.5 (C), 132.6 (CH), 132.8 (C), 135.3 (C), 138.0 (C), 154.1 (C), 163.6 (C), 165.0 (CO), 172.1 (CO). IR Spectrum; evaporated film: v˜(cm-1)=3628, 3422, 3057, 2920, 1676, 1623, 1532, 1446, 1359, 1320, 1260, 1130. MSES (negative): 481.2 (M−H+), 483.1 (M−H+). MS-ES (positive): 483.1 (M+H+), 485.1 (M+H+). HPLC: 14.313 min, 98.4% purity.

### Pharmacological Activity Experiments

Pharmacological Activity Experiments will enable selection of lead compounds for further development in animal models of acute (e.g. stroke) and/or chronic (e.g. Alzheimer's Disease) neurodegenerative disorders.

### Determination of the Capability of the Compound to Bind to PPAR-γ and CB2 Receptors

In vitro screening for PPAR-γ activity of the compounds in cell-based assays; comparative Potencies and Selectivity of the compounds in inducing PPAR-γ activation in THP-1 xderived macrophages employing a cell-based transcriptional factor assay.

The prototypic activity of PPARs is to activate transcription in a ligand-dependent manner following direct binding to DNA response elements in the promoter or enhancer regions of target genes—the so called DR-1 elements or PPAR Response elements (PPREs)—a process known as ligand dependant trans-activation. PPARs, like other nuclear receptor family members, contain both a ligand binding domain, directing specific interaction with the cognate ligand, and a DNA-binding domain that mediates binding to specific PPREs in the regulatory/promoter domains. In response to ligand binding, PPARs undergo a conformational change that facilitates:

a) the formation of a heterodimeric complex with another ligand-activated nuclear receptor retinoid X receptor (RXR);
b) high affinity interactions with co-activators (i.e. the NCor-containing co-repressor complexes are dismissed and are replaced with co-activator complexes) that remodel chromatin and activate the cellular transcription machinery inducing PPAR transactivation of the target genes.

Thus, the rate of transcriptional activation of genes that contain PPREs is increased and their mRNA levels are elevated.

As a consequence cell-based PPAR transactivation assays were first performed to address:

a) whether the newly synthesized compounds bind/activate PPAR-γ in biological systems;
b) the biological potency and PPAR selectivity of the compounds, in comparison to known PPAR-γ ligands;
c) their effects on cell viability at biologically active concentrations by determining, in addition to cell viability, PPAR DNA binding activity in nuclear extracts of THP-1 human monocytic cells differentiated into macrophage-like cells exposed to different concentrations of the compounds.

In addition, because PPAR subtypes share a high level of sequence and structural similarity, the nuclear receptor selectivity of the compounds found to activate PPAR-γ were tested for effects on PPAR-α and -δ.

Selection to employ THP-1 derived macrophages was based on the following criteria:

a) THP-1 cells differentiated towards macrophages employing phorbol esters express high levels of PPAR-γ;
b) THP-1 cells also express PPAR-α and PPAR-δ;
c) THP-1 cells have been widely employed to assess biological effects of PPAR-γ and PPAR-α agonists in monocytes/macrophages (see next step);
d) THP-1 derived macrophages have been employed for drug screening purposes of PPAR-γ agonists employing immunoabsorbent(Elisa)-based transcriptional factor assays.

Briefly, THP-1 monocytes (ATCC) in culture were treated with PMA (400 ng/mL) for 72 hours to induce monocyte differentiation into macrophages. Thereafter, test compounds at different concentrations (0.01 to 50 uM), selective PPAR-γ agonists (e.g. rosiglitazone, positive control) or vehicle (0.1% DMSO) with or without the PPAR-γ antagonist GW9662 (5 μM, 1 h prior to the samples), were added and incubated for 48 h in culture medium and nuclear extracts employed for assessment of PPAR-γ activation. At all times, cell viability, employing MTT assay, were assessed. The activation of PPAR-γ was determined by an immunosorbent assay (ELISA) utilizing PPAR-γ factor transcription factor assay kits (e.g. Cayman chemicals, USA), whilst the PPAR complete transcription factor assay kit (Cayman Chemicals) was employed for assessment of effects on PPARα and δ, of the active compounds. Comparative potencies were be determined in terms of fold activation at different concentrations.

Screening for CB2 Receptor Binding Affinity, Selectivity and Potency of the Newly Synthesized Compounds

To assess the capability of the compounds to bind to CB2 receptors and to behave as agonists/inverse agonists at CB2 receptors, the following experimental in vitro paradigms will be employed:

a) In vitro binding assays to exploit CB2 receptor affinity and selectivity off the newly-synthesized compounds via testing of their ability to selectively displace binding of [3H]-CP55,940 to membrane preparations expressing recombinant human CB2 receptor versus membrane preparations expressing recombinant human CB1 receptors. [³H]CP55940 is the most widely used radio-labelled CB1/2 receptor probe. It has approximately equal affinity for CB1 and CB2 binding sites and displacement assays with [3H]CP55940 that are directed at characterizing the binding properties of novel unlabeled ligands are generally performed with membranes that are known to contain either CB1 or CB2 receptors but not both receptor types. These membranes are often obtained from CHO cells transfected with CB1 or CB2 receptors (hCB1/2-CHO).
b) In vitro functional bioassays to exploit relative capability of selected compounds to inhibit forskolin-induced stimulation of cyclic AMP production in cells transfected with CB2 receptors (e.g. hCB2-CHO cells). CB2 receptors are negatively coupled to adenylyl cyclase and the ability of cannabinoid CB1/2 receptor agonists to inhibit basal or forskolin-induced cyclic AMP production is widely exploited for functional assessment of ligand receptor binding potency in vitro. Assays will be performed utilizing existing procedures and different concentrations of the compounds. Intracellular cAMP in cellular lysates will be measured by cAMP enzyme immunoassays techniques.
c) In vitro functional bioassays to exploit effects of selected compounds on the coupling of CB2 receptors to G proteins via assessment of their effects on the binding of [[35^{S}]GTRγS to recombinant cell membranes expressing CB2 receptors (e.g. hCB1/2-CHO). Although this assay is less sensitive than the cyclic AMP assay, it provides a total measure of G protein-mediated cannabinoid receptor activation rather than a measure of the activation of just one particular cannabinoid receptor effector mechanism as in the cyclic AMP assay. In general, it is expected that the binding of GTRγS to G proteins wouls be stimulated by agonists for G protein-coupled receptors and inhibited by inverse agonists for such receptors. In brief, in these experiments, membranes were incubated in the presence of absence of different concentrations of the compounds, [[35^{S}]GTPγS will be assessed.

### Pharmacological Activity Experiments Results

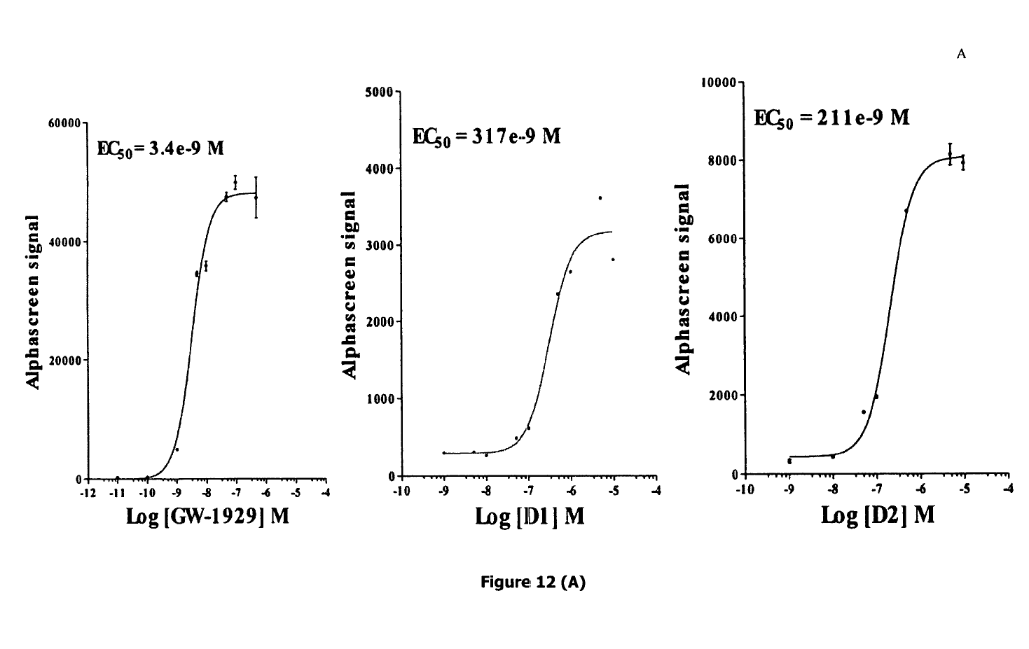

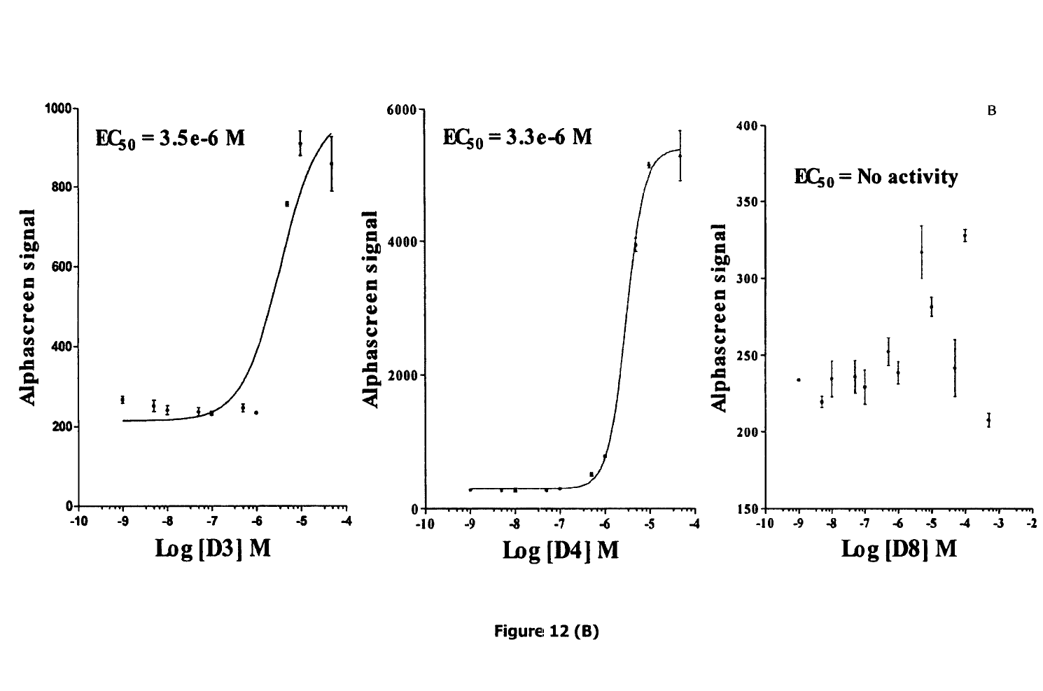

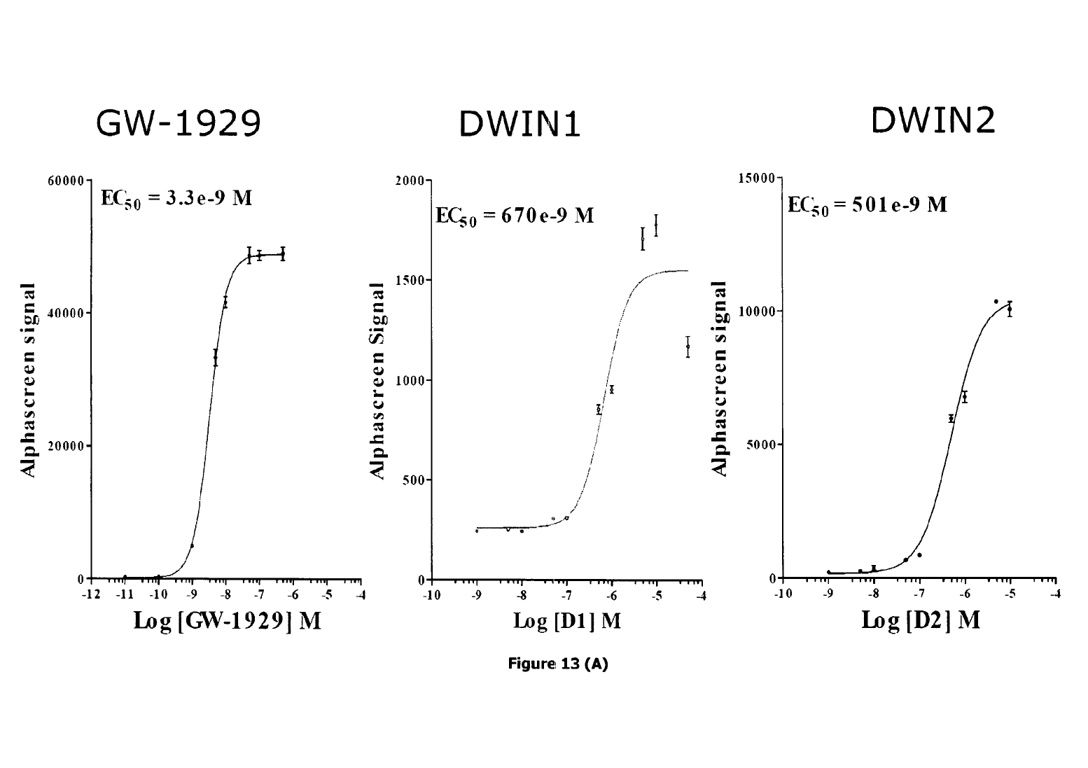

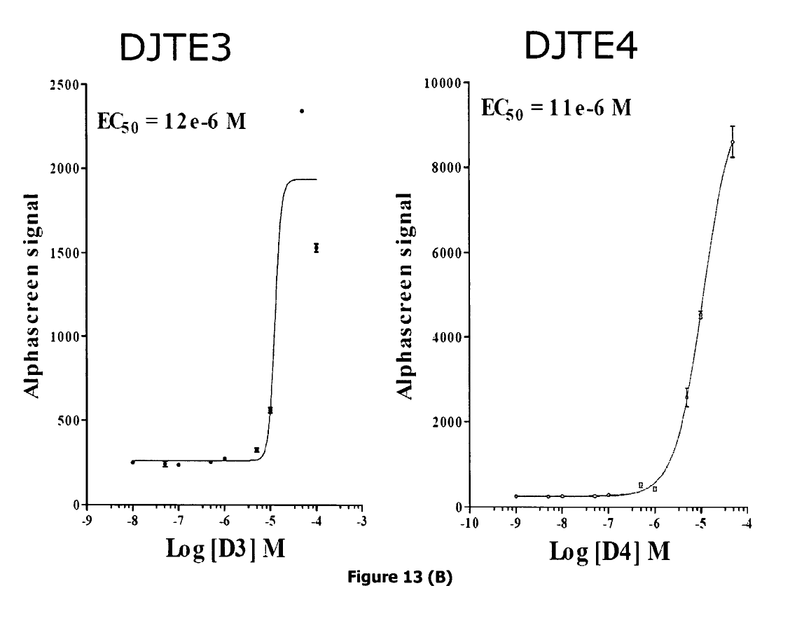

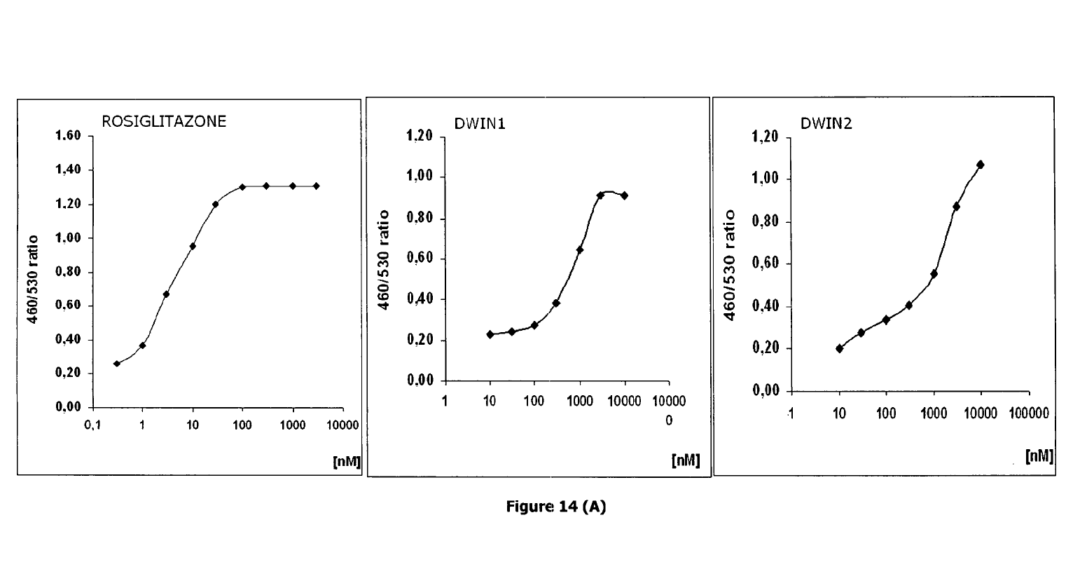

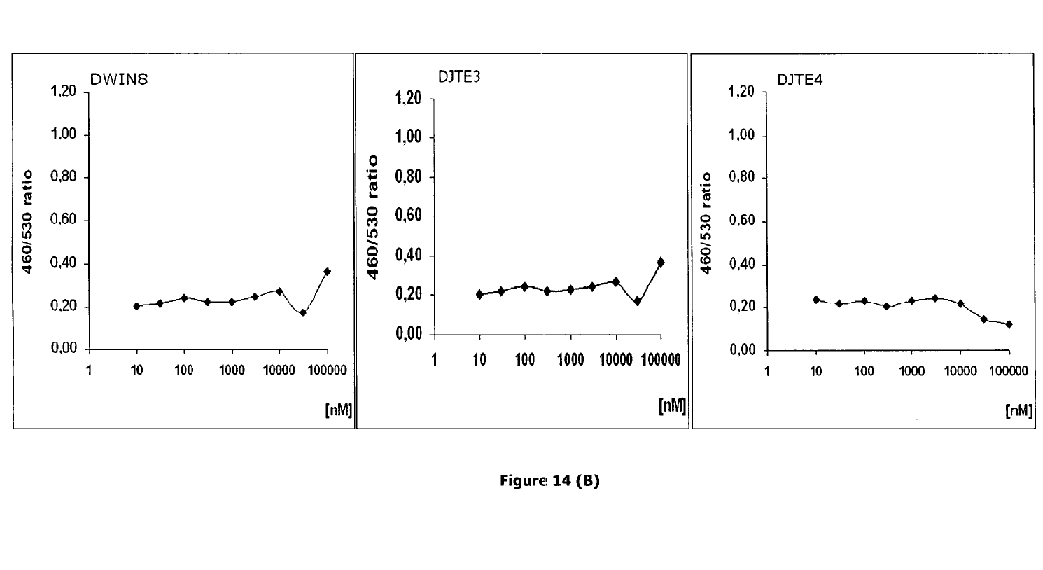

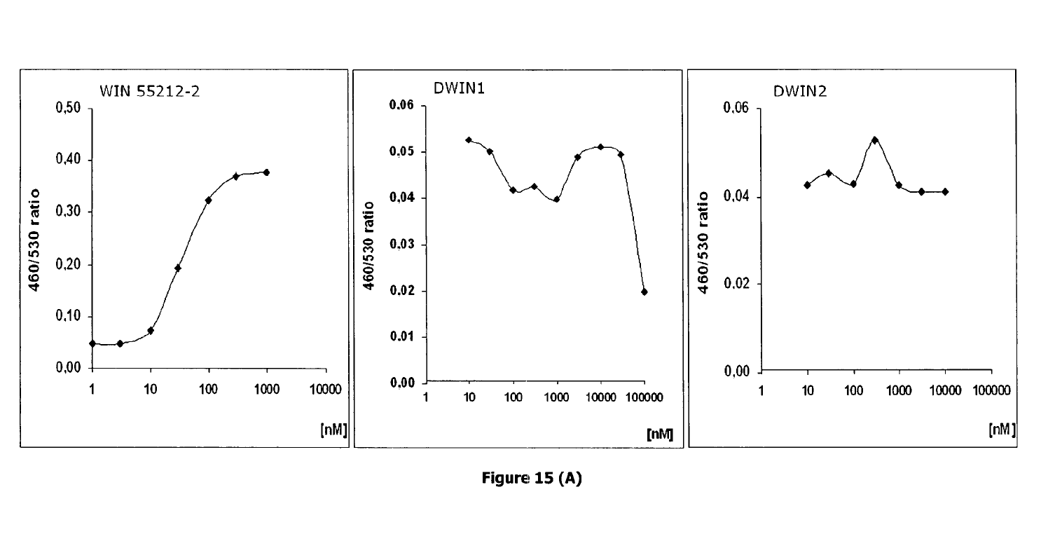

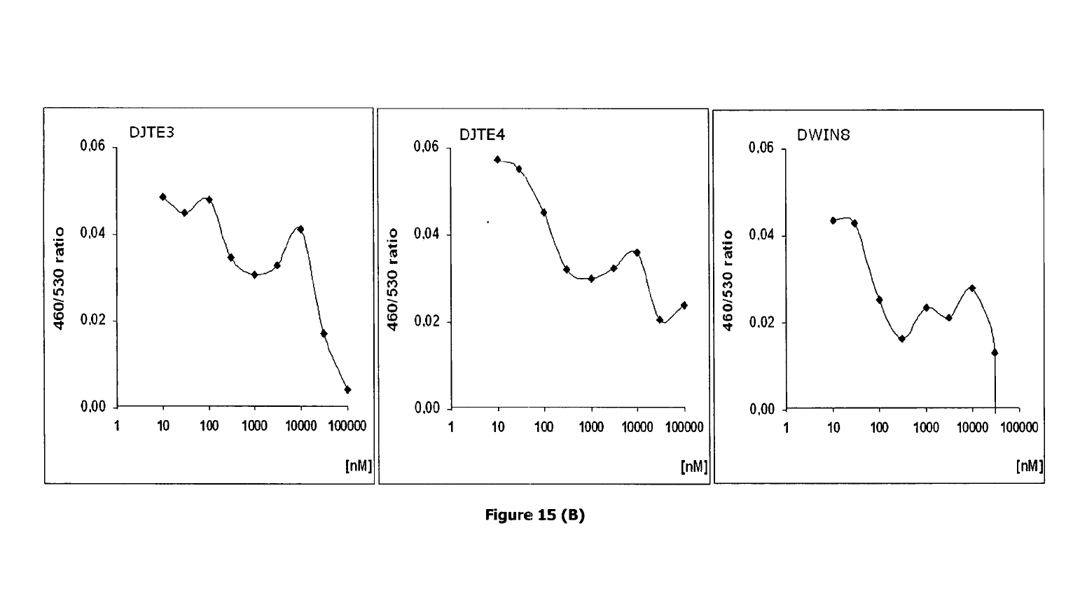

The tables set out the results obtained from the initial dose-response curves shown in FIGS. 12-15. The results in Table 1 are the average EC50 determined in duplicate as shown in FIGS. 12 and 13. FIGS. 14 and 15 show the results for tests in cell based systems for DWIN1 and DWIN2 versus rogiglitazone as control and the results for the CB2 control WIN 55212-2. Comparison of the half maximal effective concentration (EC₅₀) shows that for the PPAR-γ receptor the tested compounds are substantially more potent than the GW1929 high affinity agonist of PPAR-γ γ sold by Sigma Aldrich. The potency is dramatically higher in the cell free and cell based tests.

TABLE 1
Activity PPAR-γ - Cell Free
Activity PPAR-γ - Cell Free

| Compound | EC50 (nM) |
|---|---|
| GW1929 | 3.4 |
| DWIN1 (IX) | 493 |
| DWIN2 (X) | 358 |
| DJTE3 (XIX) | 7750 |
| DJTE4 (XX) | 7150 |
| DWIN8 (XII) | nd |

Whereas FIG. 15 initial dose-response curves suggests that DWIN8(XII) is not active, it is believe that the compound will be active at a higher dose.

TABLE 2
Activity PPAR-γ - Cell based system (GeneBlazer)
Activity PPAR-γ - Cell based system (GeneBlazer)

| Compound | EC50 (nM) |
|---|---|
| Rosiglitazone | 4 |
| DWIN1 (IX) | 800 |
| DWIN2 (X) | 1050 |
| DJTE3 (XIX) | Nd |
| DJTE4 (XX) | Nd |
| DWIN8 (XII) | Nd |

TABLE 3
Activity CB2 Cell based system (GeneBlazer)
Activity CB2 Cell based system (GeneBlazer)

| Compound | EC50 (nM) |
|---|---|
| WIN 55212-2 | 21 |
| DWIN1 (IX) | Nd |
| DWIN2 (X) | Nd |
| DJTE3 (XIX) | Nd |
| DJTE4 (XX) | Nd |
| DWIN8 (XII) | Nd |

These studies reinforce the preliminary results obtained during the modelling studies insofar as the Goldscore docking studies indicated higher docking scores for PPAR binding.

Similarly, the Goldscore docking studies for the CB2 receptor indicated that the affinity for the receptor was comparable to that of the control compound WIN 55212-2. On this basis it is expected that the compounds of the invention tested will be at least as potent as the control compound in the cell free and cell based systems experiments to be conducted.

### REFERENCES

(1) Eun, C. S.; Han, D. S.; Lee, S. H.; Paik, C. H.; Chung, Y. W.; Lee, J.; Hahm, J. S. Attenuation of colonic inflammation by PPARγ in intestinal epithelial cells: effect on Toll-like receptor pathway. Digestive Diseases and Sciences 2006, 51, 693-697.
(2) Rousseaux, C.; Thuru, X.; Gelot, A.; Barnich, N.; Neut, C.; Dubuquoy, L.; Dubuquoy, C.; Merour, E.; Geboes, K.; Chamaillard, M.; Ouwehand, A.; Leyer, G.; Carcano, D.; Colombel, J.-F.; Ardid, D.; Desreumaux, P. Lactobacillus acidophilus modulates intestinal pain and induces opioid and cannabinoid receptors. Nature Medicine 2007, 13, 35-37.
(3) Lo Verme, J.; Russo, R.; La Rana, G.; Fu, J.; Farthing, J.; Mattace-Raso, G.; Meli, R.; Hohmann, A.; Calignano, A.; Piomelli, D. Rapid broad-spectrum analgesia through activation of peroxisome proliferator-activated receptor-a. Journal of Pharmacology and Experimental Therapeutics 2006, 319, 1051-1061.
(4) Adachi, M.; Kurotani, R.; Morimura, K.; Shah, Y.; Sanford, M.; Madison, B. B.; Gumucio, D. L.; Marin, H. E.; Peters, J. M.; Young, H. A.; Gonzalez, F. J. Peroxisome proliferator activated receptor γ in colonic epithelial cells protects against experimental inflammatory bowel disease. Gut 2006, 55, 1104-1113.
(5) Dubuquoy, L.; Rousseaux, C.; Thuru, X.; Peyrin-Biroulet, L.; Romano, O.; Chavatte, P.; Chamaillard, M.; Desreumaux, P. PPARγ as a new therapeutic target in inflammatory bowel diseases. Gut 2006, 55, 1341-1349.
(6) Wotherspoon, G.; Fox, A.; McIntyre, P.; Colley, S.; Bevan, S.; Winter, J. Peripheral nerve injury induces cannabinoid receptor 2 protein expression in rat sensory neurons. Neuroscience 2005, 135, 235-245.
(7) Burstein, S. H. PPAR-γ: A nuclear receptor with affinity for cannabinoids. Life Sciences 2005, 77, 1674-1684.
(8) Burstein, S. H.; Karst, M.; Schneider, U.; Zurier, R. B. Ajulemic acid: a novel cannabinoid produces analgesia without a “high”. Life Sciences 2004, 75, 1513-1522.
(9) Russo, R.; LoVerme, J.; La Rana, G.; D'Agostino, G.; Sasso, O.; Calignano, A.; Piomelli, D. Synergistic antinociception by the cannabinoid receptor agonist anandamide and the PPAR-a receptor agonist GW7647. European Journal of Pharmacology 2007, 566, 117-119.
(10) Wright, K.; Rooney, N.; Feeney, M.; Tate, J.; Robertson, D.; Welham, M.; Ward, S. Differential expression of cannabinoid receptors in the human colon: cannabinoids promote epithelial wound healing. Gastroenterology 2005, 129, 437-453.
(11) Zimmermann, G. R.; Lehar, J.; Keith, C. T. Multi-target therapeutics: when the whole is greater than the sum of the parts. Drug Discovery Today 2007, 12, 34-42.
(12) Morphy, R.; Kay, C.; Rankovic, Z. From magic bullets to designed multiple ligands. Drug Discovery Today 2004, 9, 641-651.
(13) Morphy, R.; Rankovic, Z. Designed multiple ligands. An emerging drug discovery paradigm. Journal of Medicinal Chemistry 2005, 48, 6523-6543.
(14) Wermuth, C. G. Multitargeted drugs: the end of the ‘one-target-onedisease’ philosophy? Drug Discovery Today 2004, 9, 826-827.
(15) Keith, C. T.; Borisy, A. A.; Stockwell, B. R. Multicomponent therapeutics for networked systems. Nature Reviews Drug Discovery 2005, 4, 1-8.
(16) Cronet, P.; Petersen, J. F. W.; Folrner, R.; Blornberg, N.; Sjöblom, K.; Karlsson, U.; Lindstedt, E.-L.; Bamberg, K. Structure of the PPARa and -γ ligand binding domain in complex with AZ 242; ligand selectivity and agonist activation in the PPAR family. Structure 2001, 9, 699-706.
(17) Berman, H. M.; Westbrook, J.; Feng, Z.; Gary, G.; Bhat, T. N.; Weissig, H.; Shindyalov, I. N.; Bourne, P. E. The protein data bank. Nucleic Acids Research 2000, 28, 235-242.
(18) Stern, E.; Muccioli, G. G.; Millet, R.; Goossens, J.-F.; Farce, A.; Chavatte, P.; Poupaert, J. H.; Lambert, D. M.; Depreux, P.; Henichart, J.-P. Novel 4-oxo-1,4-dihydroquinoline-3-carboxamide derivatives as new CB₂ cannabinoid receptors agonists: synthesis, pharmacological properties and molecular modeling. Journal of Medicinal Chemistry 2006, 49, 70-79.
(19) Raitio, K. H.; Salo, O. M. H.; Nevalainen, T.; Poso, A.; Jarvinen, T. Targeting the cannabinoid CB2 receptor: mutations, modeling and development of CB2 selective ligands. Current Medicinal Chemistry 2005, 12, 1217-1237.
(20) Raitio, K. H.; Savinainen, J. R.; Vepsalainen, J.; Laitinen, J. T.; Poso, A.; Jarvinen, T.; Nevalainen, T. Synthesis and SAR studies of 2-oxoquinoline derivatives as CB2 receptor inverse agonists. Journal of Medicinal Chemistry 2006, 49, 2022-2027.
(21) Dyson, A.; Peacok, M.; Chen, A.; Courade, J.-P.; Yaqob, M.; Groarke, A.; Brain, C.; Loong, Y.; Fox, A. Antihyperalgesic properties of the cannabinoid CT-3 in chronic neuropathic and inflammatory pain states in the rat. Pain 2005, 116, 129-137.
(22) Liu, J.; Li, H.; Burstein, S. H.; Zurier, R. B.; Chen, J. D. Activation and binding of peroxisome proliferator-activated receptor g by synthetic cannabinoid ajulemic acid. Molecular Pharmacology 2003, 63, 983-992.
(23) Kresge, A. J.; Lough, A. J.; Zhu, Y. 2-Hydroxycyclohexene-1-carboxylic acid: the enol isomer of 2-oxocyclohexane-1-carboxylic acid. Acta Crystallographica Section E 2003, 59, 344-346.
(24) Iwamura, H.; Suzuki, H.; Ueda, Y.; Kaya, T.; Inaba, T. In vitro and in vivo pharmacological characterization of JTE-907, a novel selective ligand for cannabinoid CB₂ receptor. Journal of Pharmacology and Experimental Therapeutics 2001, 296, 420-425.
(25) SYBYL; 6.9.1 ed.; Tripos Associates Inc., 1699 South Hanley Road, St. Louis, Mo. 63144.
(26) Hilal, S. H.; Karickhoff, S. W.; Carreira, L. A. A rigorous test for SPARC's chemical reactivity models: estimation of more than 4300 ionization pKas. Quantitative Structure-Activity Relationships 1995, 14, 348-355.
(27) Clark, M.; Cramer III, R. D.; Van Opdenbosch, N. Validation of the general purpose Tripos 5.2 force field. Journal of Computational Chemistry 1989, 10, 982-1012.
(28) Munro, S.; Thomas, K. L.; Abu-Shaar, M. Molecular characterization of a peripheral receptor for cannabinoids. Nature 1993, 365, 61-65.
(29) Nathans, J.; Hogness, D. S. Isolation, sequence analysis, and intron-exon arrangement of the gene encoding bovine rhodopsin. Cell 1983, 34, 807-814.
(30) Thompson, J. D.; Higgins, D. G.; Gibson, T. J. CLUSTAL W: improving the sensitivity of progressive multiple sequence alignment through sequence weighting, position-specific gap penalties and weight matrix choice. Nucleic Acids Research 1994, 22, 4673-4680.
(31) Okada, T.; Fujiyoshi, Y.; Silow, M.; Navarro, J.; Landau, E. M.; Shichida, Y. Functional role of internal water molecules in rhodopsin revealed by x-ray crystallography. Proceedings of the National Academy of Sciences 2002, 99, 5982-5987.
(32) Petrey, D.; Xiang, Z.; Tang, C. L.; Xie, L.; Gimpelev, M.; Mitros, T.; Soto, C. S.; Goldsmith-Fischman, S.; Kernytsky, A.; Schlessinger, A.; Koh, I. Y. Y.; Alexov, E.; Honig, B. Using multiple structure alignments, fast model building, and energetic analysis in fold recognition and homology modeling. Proteins: Structure, Function, and Genetics 2003, 53, 430-435.
(33) McAllister, S. D.; Rizvi, G.; Anavi-Goffer, S.; Hurst, D. P.; Barnett-Norris, J.; Lynch, D. L.; Reggio, P. H.; Abood, M. E. An aromatic microdomain at the cannabinoid CB₁ receptor constitutes an agonist/inverse agonist binding region. Journal of Medicinal Chemistry 2003, 46, 5139-5152.
(34) Jones, G.; Willett, P.; Glen, R. C.; Leach, A. R.; Taylor, R. Development and validation of a genetic algorithm for flexible docking. J Mol Biol 1997, 267, 727-748.

### REFERENCES FOR SYNTHETIC SECTION

[1] K. H. Raitio, J. R. Savinainen, J. Vepsalainen, J. T. Laitinen, A. Poso, T. Jarvinen, T. Nevalainen, J. Med. Chem. 2006, 49, 2022.
[2] C. D. Guillon, G. A. Koppel, M. J. Brownstein, M. O. Chaney, C. F. Ferris, S. F. Lu, K. M. Fabio, M. J. Miller, N. D. Heindel, D.C. Hunden, R. D. G. Cooper, S. W. Kaldor, J. J. Skelton, B. A. Dressman, M. P. Clay, M. I. Steinberg, R. F. Bruns, N. G. Simon, Bioorg. Med. Chem. 2007, 15, 2054.
[3] J. D. Xu, A. E. V. Gorden, K. N. Raymond, Eur. J. Org. Chem. 2004, 3244.
[4] X. Marguerettaz, R. Oneill, D. Fitzmaurice, J. Am. Chem. Soc. 1994, 116, 2629.
[5] S. W. Kang, C. M. Gothard, S. Maitra, W. Atia tul, J. S, Nowick, J. Am. Chem. Soc. 2007, 129, 1486.
[6] P. J. Palmer, R. J. Ward, Warringt. Jv, J. Med. Chem. 1971, 14, 1226.
[7] M. R. Bell, T. E. Dambra, V. Kumar, M. A. Eissenstat, J. L. Herrmann, J. R. Wetzel, D. Rosi, R. E. Philion, S. J. Daum, D. J. Hlasta, R. K. Kullnig, J. H. Ackerman, D. R. Haubrich, D. A. Luttinger, E. R. Baizman, M. S. Miller, S. J. Ward, J. Med. Chem. 1991, 34, 1099.
[8] J. H. Kang, S. Benzaria, D. M. Sigano, N. E. Lewin, Y. M. Pu, M. L. Peach, P. M. Blumberg, V. E. Marquez, J. Med. Chem. 2006, 49, 3185.
[9] D. J. Bennett, A. J. Blake, P. A. Cooke, C. R. A. Godfrey, P. L. Pickering, N. S. Simpkins, M. D. Walker, C. Wilson, Tetrahedron 2004, 60, 4491.
[10] R. A. Tapia, Y. Prieto, M. Pautet, M. Domard, M. E. Sarciron, N. Walchshofer, H. Fillion, Eur. J. Org. Chem. 2002, 4005.
[11] A. Boularot, C. Giglione, S. Petit, Y. Duroc, R. A. de Sousa, V. Larue, T. Cresteil, F. Dardel, I. Artaud, T. Meinnel, J. Med. Chem. 2007, 50, 10.
[12] C. Avendano, J. D. Sanchez, J. C. Menendez, Synlett 2005, 107. [13] S. Kubik, R. Goddard, Eur. J. Org. Chem. 2001, 311.
[14] J. Rickerby, R. Prabhakar, A. Patel, J. Knowles, S. Brocchini, J. Control. Release 2005, 101, 21.
[15] C. Gleye, G. Lewin, A. Laurens, J. C. Jullian, P. Loiseau, C. Bories, R. Hoequemiller, J. Nat. Prod. 2003, 66, 690.

1. A compound having activity at, at least one of a PPAR and a cannabinoid receptor comprising:
a PPAR pharmacophore linked to a cannabinoid pharmacophore comprising a fused bicyclic ring comprising a five membered ring fused with a six membered ring or a six membered ring fused with a six membered ring;
and
the PPAR pharmacophore is selected from the group consisting of: 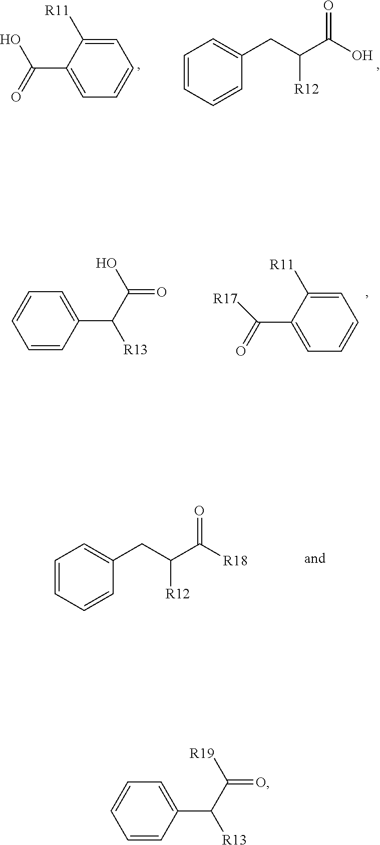
wherein R¹¹, R¹², and R¹³ are each independently selected from the group consisting of: OH, C₁-C₈ alkoxy, C₃-C₆ cycloalkoxyl, —OCH₂CH₂, C₃-C₅ allyloxyl, —OPh, naphthaloxy, —OCH₂Ph and a phenylphenoxy;
R¹⁷, R¹⁸ and R¹⁹ are each independently selected from the group consisting of: OH, C₁-C₈ alkoxy, C₃-C₆ cycloalkoxyl, OCH₂CH₂, a C₃-C₅ allyloxyl, OPh, naphthaloxy, —OCH₂Ph and a phenylphenoxy;
wherein the PPAR pharmacophore is linked to the bicyclic ring through a linker selected from the group consisting of —X′NR′—, —NR′—, —C(O)NR′—; —C(O)NR′R″—; —NR′C(O)R″—; —C(O)NR′NR″—; —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—, in which R′ and R″ is independently hydrogen, optionally substituted C₁-C₈ alkyl, C₃-C₁₀ cycloalkyl, aryl, heteroaryl, aralkyl, alkoxy or heteroaralkyl; and X′ and X″ is independently a bond, —NH—, piperzine, C₁-C₈ allyl, a C₁-C₈ alkylene or C₁-C₈ alkyl;
or a pharmaceutically acceptable salt thereof.

2-4. (canceled)

5. A compound according to claim 1 wherein the linker is selected from the group consisting of —C(O)NHNH—, —C(O)NC₂H₄N— and —C(O)NHCH₂CH₂—.

6-11. (canceled)

12. A compound according to claim 1 wherein the fused bicyclic ring is selected from the group consisting of 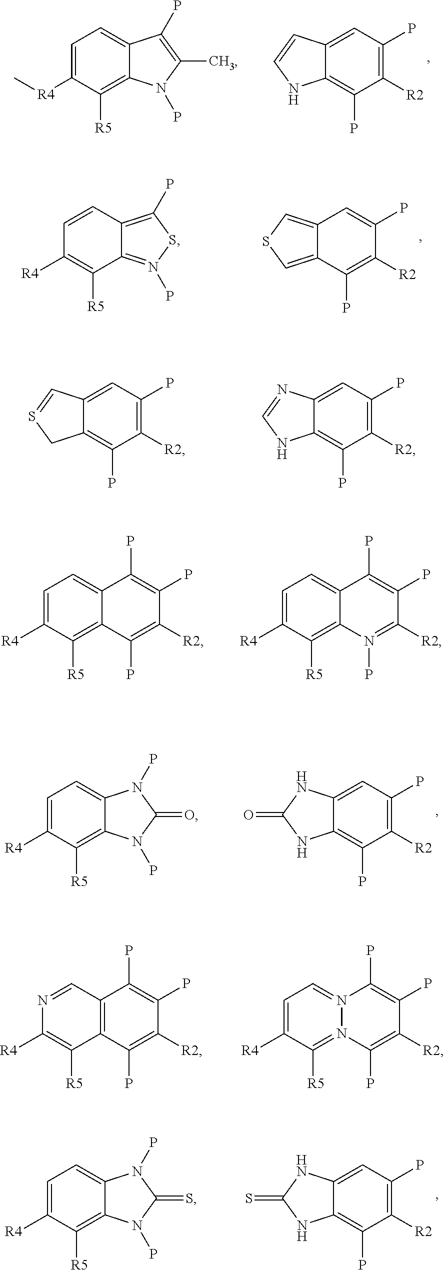 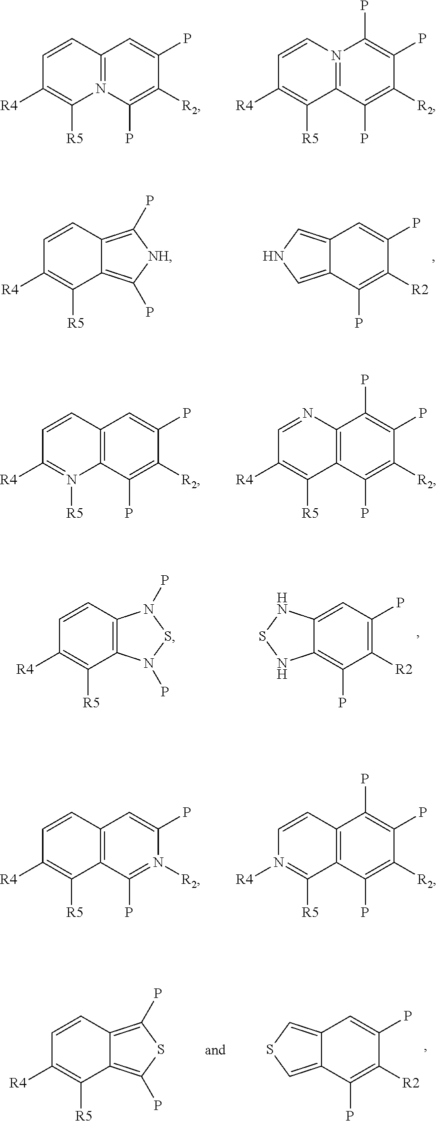
wherein P is H, the PPAR pharmacophore or the cannabinoid pharmacophore;
R₁ is H;
R₂ is H, methyl, ═O, ═S, ═NH, C₁-C₅ alkyl, C₁-C₅ alkoxy or a lone pair of electrons;
R₄ is H, methyl, ═O, ═S, ═NH, C₁-C₅ alkyl, or C₁-C₅ alkoxy;
R₅ is H, methyl, ═O, ═S, ═NH, C₁-C₅ alkyl, or C₁-C₅ alkoxy.

13-14. (canceled)

15. A compound according to claim 1 wherein the cannabinoid pharmacophore is selected from the group consisting of: 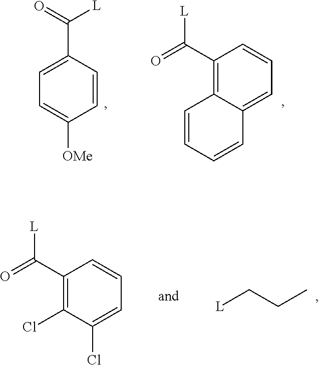
wherein L represents the fused bicyclic to which the cannabinoid pharmacophore substituent is bound.

16. A compound according to claim 1 wherein the PPAR pharmacophore and linker are selected from 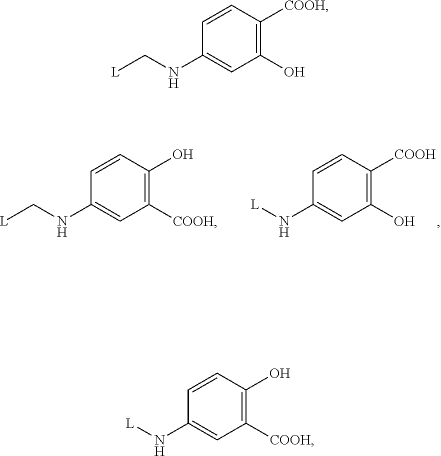
the group consisting of: 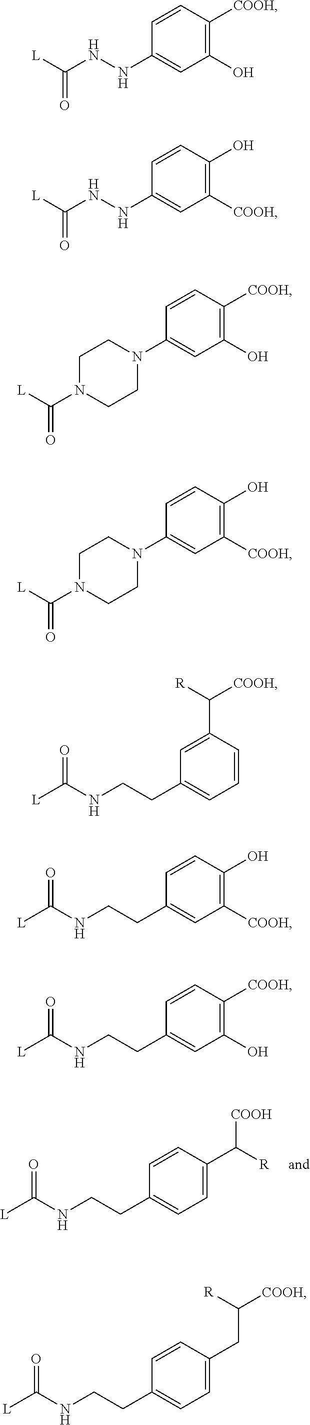
a nd wherein the fused bicylic ring is substituted by a substituent selected from the group consisting of: 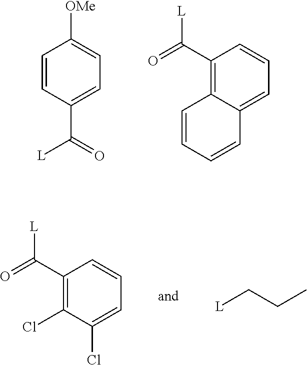
wherein L represents the fused bicycle ring to which the substituent is attached.

17-18. (canceled)

19. A compound having the general structure (II): 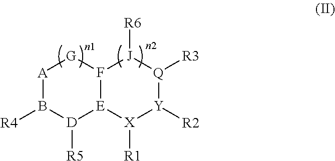
wherein at least one of the fused bicycle rings is aromatic;
n¹ is 0 or 1;
n² is 0 or 1; wherein at least one of n1 or n2 is 1;
A is CH, N or S; B is C, N or S; D is C or N; E is C or N; F is C or N; G is CH, N or S; X is C or N; Y is C, N or S; Q is C or N; J is CH, N or S; or
A is CH, N, NH or S; B is C, N or S; D is C, N or S; E is C or N; F is C or N; G is CH, N, NH or S; X is C or N; Y is C, N or S; Q is C or N; J is CH, N or NH;
and
one of R₁, R₃ or R₆ is R₁₄,
R₁₄ is selected from the group consisting of an amide or amine linkage covalently bound to a PPAR pharmacophore selected from the group consisting of: 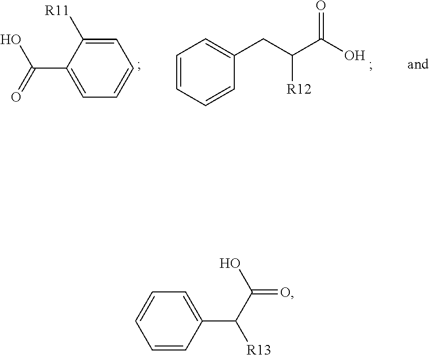
wherein:
R¹¹, R¹², and R¹³ are each independently selected from the group consisting of: OH, C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) and a phenylphenoxy (—OPhPh) group
R₁₅ is a cannabinoid pharmacophore substituent selected from the group consisting of: 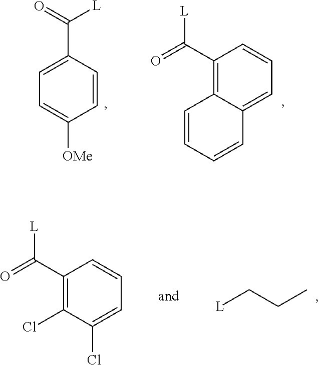
wherein L indicates the point of attachment;
R₁ selected from H, C₁-C₈ alkyl, R₁₅ or R₁₄;
R₂ is H, methyl, ═O, ═S, ═NH or a lone pair of electrons;
R₃ is H, or or is a cannabinoid pharmacophore substituent R₁₄, or R₁₅; and
R₄ is H, methyl, ═O, ═S, ═NH, C₁-C₅ alkyl or C₁-C₅ alkoxy;
R₅ is H, methyl, ═O, ═S, ═NH, C₁-C₅ alkyl or C₁-C₅ alkoxy;
R₆ is H,
R₁₄, or R₁₅;
with the proviso that,
when B is S, R₄ is a lone pair of electrons; and
when R₁ is R₁₄ then R₃ is R₁₅ and when R₃ is R₁₄ then R₁ is R₁₅
or a pharmaceutically acceptable salt thereof.

20-27. (canceled)

28. A compound according to claim 1, having general formula V*, 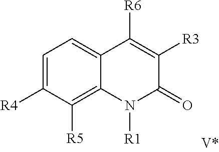
wherein
R₁ is H, or C₁-C₈ alkyl or a cannabinoid pharmacophore substituent;
R₃ is a cannabinoid pharmacophore substituent or —R₁₆-R₁₄; wherein R₁₆ is an amide or amide linker selected from the group consisting of —X′NR′—, —NR′—, —C(O)NR′R″—, —NR′C(O)R″—, —C(O)NR′NR″—, —X′NR′R″X″—, —X′NR′C(O)X″—, —X′NR′C(O)NR″X″—, —X′NR′C(O)OX″—, —X′C(O)NR′X″—, —X″R″NC(O)NR′X′— and —X″OC(O)NR′X′—, in which,
R′ is hydrogen, optionally substituted C₁-C₈ alkyl, C₃-C₁₀ cycloalkyl, aryl, heteroaryl, aralkyl, alkoxy or heteroaralkyl; R″ is optionally substituted C₁-C₈ alkyl, C₃-C₁₀ cycloalkyl, aryl, heteroaryl, aralkyl, alkoxy or heteroaralkyl; and X′ and X″ is independently a bond, —NH—, piperzine, C₁-C₈ allyl, a C₁-C₈ alkylene or C₁-C₈ alkyl; and
R₁₄ is selected from the group consisting of: 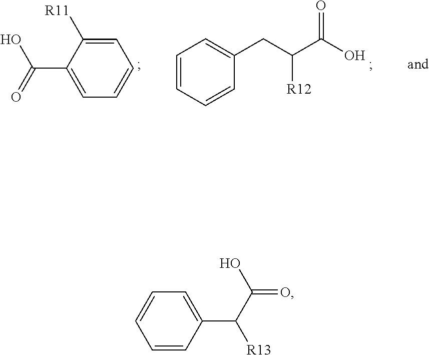
wherein:
R₁₁, R₁₂, and R₁₃ are each independently selected from the group consisting of: OH, C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) and a phenylphenoxy (—OPhPh) group;
R₄ is C₁-C₈alkoxy, C₁-C₈alkyl or H;
R₅ is H, methyl, ═O, ═S or NH, C₁-C₅ alkyl or C₁-C₅ alkoxy;
R₆ is H or a cannabinoid pharmacophore substituent.

29. A compound according to claim 28 wherein the cannabinoid pharmacophore substituent is selected from the group consisting of: 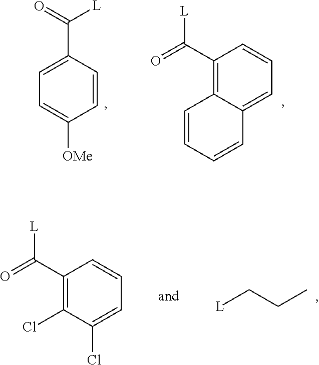
wherein L represents the fused bicycle ring to which the substituent is attached.

30. A compound d according to claim 1 having general formula (VI) or (VII): 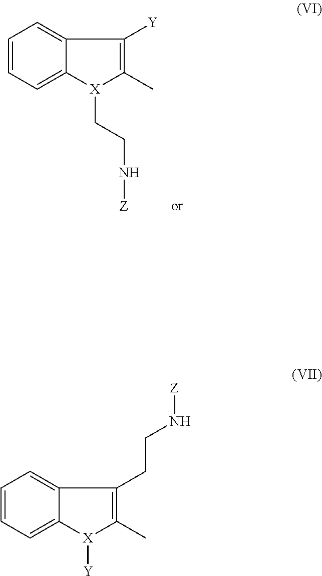
wherein
X is C, N or S; and
Y is a naphthoyl, arylcarboxy, cycloalkylcarboxy, arylcarbamoyl, cycloalkylcarbamoyl or alkylcarbamoyl group; and
Z is a salicylic acid functionality, an alkoxybenzylacetic acid functionality or an alkoxyphenylacetic acid functionality wherein Z may be substituted at the PPAR pharmacophore carboxylic acid OH group, wherein the OH is substituted with a C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) and a phenylphenoxy (—OPhPh) group.

31. (canceled)

32. A compound according to claim 1 selected from the group consisting of: 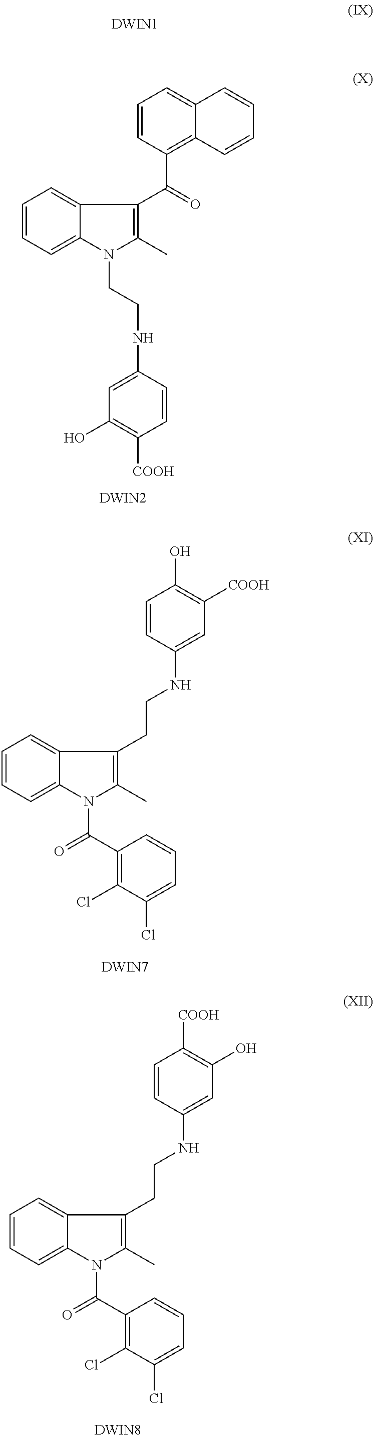 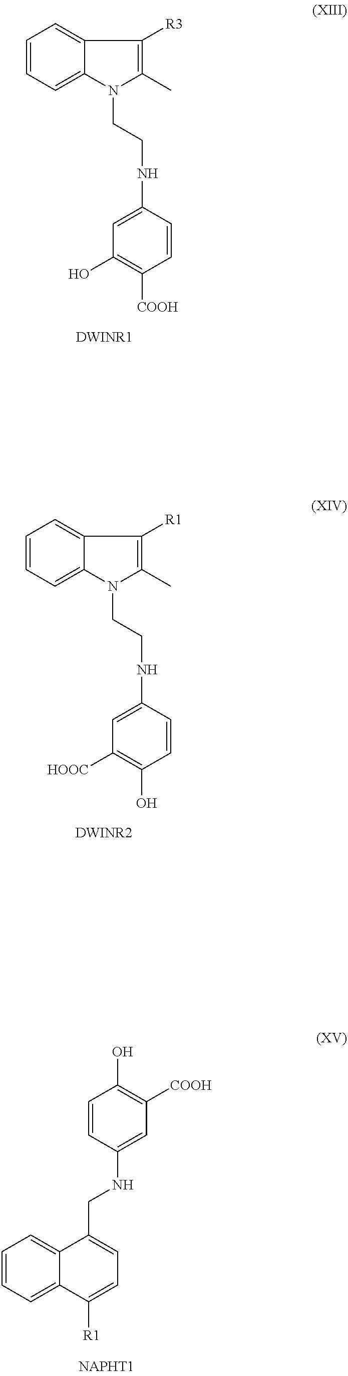 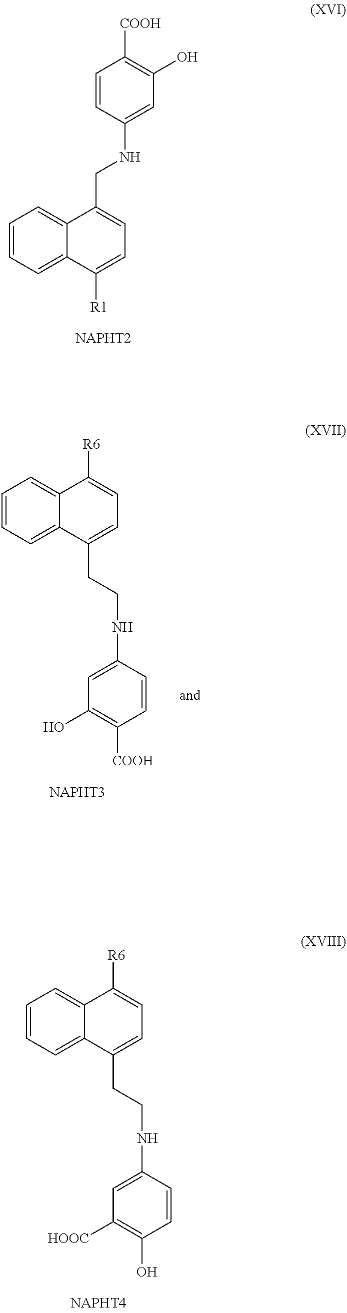
wherein R₁, R₃, and R₆ is a arylcarboxy, cycloalkylcarboxy, alkylcarboxy, arylcarbamoyl, cycloalkylcarbamoyl or a alkylcarbamoyl group, 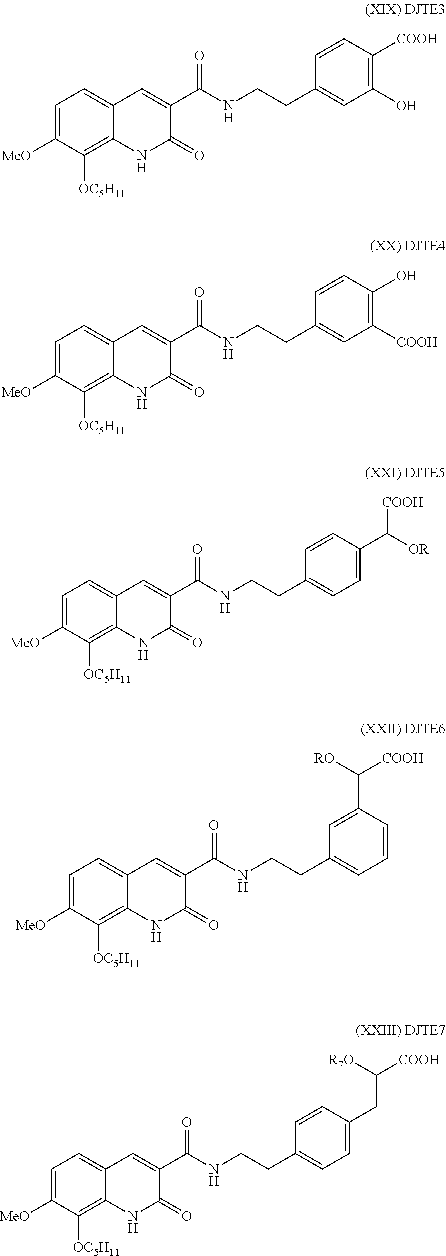 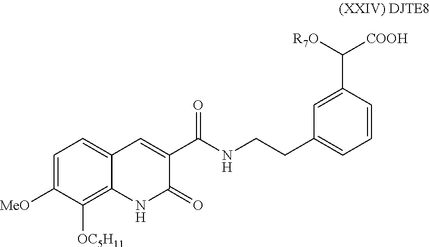
wherein —OR₇ is OH, a C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) and a phenylphenoxy (—OPhPh) group.

33. A compound, having general formula (VIII) 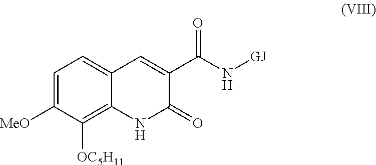
wherein
G is a C₁-C₃ alkyl group; and
J is a salicylic acid functionality or an alkoxybenzylacetic acid functionality or an alkoxyphenylacetic acid functionality, wherein J may further comprise a substitution at the PPAR pharmacophore carboxylic acid OH group, wherein the OH is substituted with a C₁-C₈ alkoxy, C₃-C₆ cycloalkoxyl (—OR^{alk}(cyc)) group, a vinyloxyl (—OCH₂CH₂), a C₃-C₅ allyloxyl, benzoxy (—OPh), naphthaloxy (—ONp), benzyloxy (—OCH₂Ph) and a phenylphenoxy (—OPhPh) group

34-37. (canceled)

38. A method of treating chronic inflammatory diseases in a patient in need thereof, wherein the chronic inflammatory disease is selected from the group consisting of Crohn's disease and ulcerative rectocolitis.

39-40. (canceled)

41. A pharmaceutical composition comprising one or more compounds according to claim 32 as active principles in combination with one or more pharmaceutically acceptable excipients or adjuvants.

42-50. (canceled)

51. The compound of claim 19, wherein n¹ is 1 and n² is 0.

52. The compound of claim 52, wherein A is CH, D is C, E is C, F is C, and G is CH.

53. The compound of claim 53, wherein B is C.

54. The compound of claim 54, wherein R₄ and R₅ is H.

55. The compound of claim 52, wherein X is N.

56. A compound selected from the group consisting of: 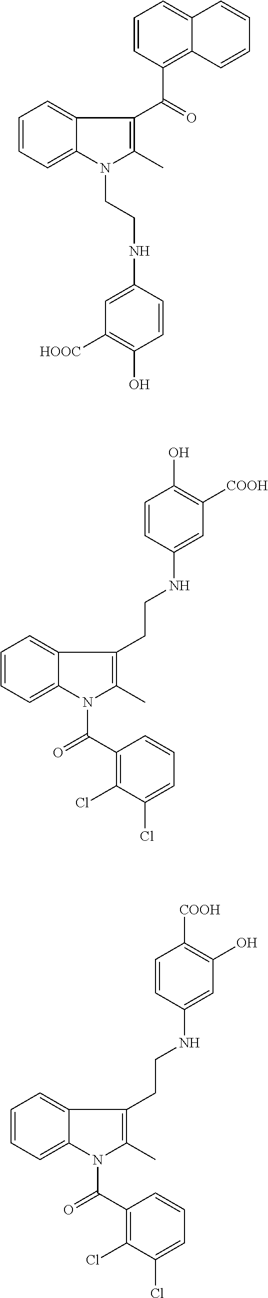
and pharmaceutically acceptable salts thereof.
